SEQUENCE LISTING (120 sequences)

SEQ ID NO: 1 - protein, 907 aa, Homo sapiens
MDTSRLGVLLSLPVLLQLATGGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQSLRLDANHISYVPPSCFSGLHSLRHLWLDDNALTEIPVQAFRSLSALQAMTLALNKIHHIPDYAFGNLSSLVVLHLHNNRIHSLGKKCFDGLHSLETLDLNYNNLDEFPTAIRTLSNLKELGFHSNNIRSIPEKAFVGNPSLITIHFYDNPIQFVGRSAFQHLPELRTLTLNGASQITEFPDLTGTANLESLTLTGAQISSLPQTVCNQLPNLQVLDLSYNLLEDLPSFSVCQKLQKIDLRHNEIYEIKVDTFQQLLSLRSLNLAWNKIAIIHPNAFSTLPSLIKLDLSSNLLSSFPITGLHGLTHLKLTGNHALQSLISSENFPELKVIEMPYAYQCCAFGVCENAYKISNQWNKGDNSSMDDLHKKDAGMFQAQDERDLEDFLLDFEEDLKALHSVQCSPSPGPFKPCEHLLDGWLIRIGVWTIAVLALTCNALVTSTVFRSPLYISPIKLLIGVIAAVNMLTGVSSAVLAGVDAFTFGSFARHGAWWENGVGCHVIGFLSIFASESSVFLLTLAALERGFSVKYSAKFETKAPFSSLKVIILLCALLALTMAAVPLLGGSKYGASPLCLPLPFGEPSTMGYMVALILLNSLCFLMMTIAYTKLYCNLDKGDLENIWDCSMVKHIALLLFTNCILNCPVAFLSFSSLINLTFISPEVIKFILLVVVPLPACLNPLLYILFNPHFKEDLVSLRKQTYVWTRSKHPSLMSINSDDVEKQSCDSTQALVTFTSSSITYDLPPSSVPSPAYPVTESCHLSSVAFVPCL

SEQ ID NO: 2 - protein, 1210 aa, Homo sapiens [sequence omitted]

SEQ ID NO: 3 - protein, 122 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAKDRHWHWWLDAFDYWGQGTLVTVSS

SEQ ID NO: 4 - protein, 120 aa, synthetic construct
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSS

SEQ ID NO: 5 - protein, 125 aa, synthetic construct
QVQLVQSGAEVKKPGASVKVSCKVSGYTLTELSMHWVRQAPGKGLEWMGGFDPEYGKTFFAQNFQGRVTMTEDTSADTAYMELSSLRSEDTAVYYCATEGYYETTTYYYNLFDSWGQGTLVTVSS

SEQ ID NO: 6 - protein, 119 aa, synthetic construct
QVQLVQSGSELKKPGASVKVSCKTSGYTFTDYAMTWVRQAPGQGLEWMGWITTNTGDPTYAPGFTGRFVFSLDTSVSTAYLQISSLKAEDTAVYYCARVYHWIRGFEFWGQGTLVTVSS

SEQ ID NO: 7 - protein, 126 aa, synthetic construct
QVQLQESGPGLVKPSETLSLTCTVSGGSFSSSSSYWGWIRQPPGKGLEWIGSFYYSGNTYYNPSLKSRVTISEDTSKNQFSLKLSSVTAADTAVYYCARQTYSSSWDGVLYYFDYWGQGTLVTVSS

SEQ ID NO: 8 - protein, 126 aa, synthetic construct
QVQLQESGPGLVKPSETLSLTCTVSNGSISTYYWSWIRQPPGKGLEWIGYVYYTGRTKYNPSLKSRVTISVDTSKNQFSLNLSSVTAADTAVYYCARGGIVVVPAARDYYYYMDVWGKGTTVTVSS

SEQ ID NO: 9 - protein, 120 aa, synthetic construct
EVQLVQSGAEVKKPGESLKIACKGSGFSFTSHWIGWVRQKPGRGLEWMGVIYPGDSDTRYSPSFQGQVTVSADKSINTAYLQWNSLKASDTAIYYCARPNSGSPRYFEFWGRGTLVTVSS

SEQ ID NO: 10 - protein, 126 aa, synthetic construct
QVQLQESGPGLVKPSETLSLTCTVSGGSISSSSYYWGWIRQPPGKGLEWIGSFYYSGNTYYNPSLKSRVTISVDTSKNQFSLKLSSVTAADTAVYYCARQEYYYGSGSPSYYFDYWGQGTLVTVSS

SEQ ID NO: 11 - protein, 120 aa, synthetic construct
EVQLVQSGAEVKKPGESLKISCKGSGDSFISHWIAWVRQMPGKGLEWMGIVYPGDSDTRYSPSFQGQVTISADKSITTAYLQWSSLKASDTAMYYCARHEWELLGPFDYWGQGTLVTVSS

SEQ ID NO: 12 - protein, 121 aa, synthetic construct
EVQLVQSGAEVKKPGSSVKVSCKASGGTSTNDAISWVRQTPGQGLEWMGSIIPILDTTDHAQKFQGRVTITADKSTNTAYMELNSLRSDDTAVYYCAREHIAARQDYFDYWGQGTLVTVSS

SEQ ID NO: 13 - protein, 127 aa, synthetic construct
EVQLVQSGSKLKKPGASVKVSCKASGYTFTSYTMNWVRQAPGQGLEWMGWINTDTGDPTYAQGFTGRFVFSLDTSVSTAFLQINSLKAEDTAVYYCARGDCDSTSCYRYSYGYEDYWGQGTLVTVSS

SEQ ID NO: 14 - protein, 119 aa, synthetic construct
QVQLVQSGAEVKKPGSSVKVSCKVSGGTFRSYAISWVRQAPGQGLEWMGGIIPIFDTRNYAQILQGRVTITADLSTSTAYMELNSLRSEDTAIYYCARGSDEGDWFDPWGQGTLVTVSS

SEQ ID NO: 15 - protein, 121 aa, synthetic construct
EVQLVQSGTEVRKPGSSVKVSCKASGGTFSNYAISWVRQAPGQGLEWMGSIIPILGTTDHAQKFQDRVTITADKSSNTTYMELSSLRSDDTAVYYCAREYIAARLDYFDSWGQGTLVTVSS

SEQ ID NO: 16 - protein, 30 aa, synthetic construct
QVQLVQSGAEVKKPGASVKVSCKASGYTFT

SEQ ID NO: 17 - protein, 5 aa, synthetic construct
SYGIS

SEQ ID NO: 18 - protein, 14 aa, synthetic construct
WVRQAPGQGLEWMG

SEQ ID NO: 19 - protein, 17 aa, synthetic construct
WISAYNGNTNYAQKLQG

SEQ ID NO: 20 - protein, 32 aa, synthetic construct
RVTMTTDTSTSTAYMELRSLRSDDTAVYYCAK

SEQ ID NO: 21 - protein, 13 aa, synthetic construct
DRHWHWWLDAFDY

SEQ ID NO: 22 - protein, 11 aa, synthetic construct
WGQGTLVTVSS

SEQ ID NO: 23 - protein, 30 aa, synthetic construct
QVQLVQSGSELKKPGASVKISCKASGYDFT

SEQ ID NO: 24 - protein, 5 aa, synthetic construct
NYAMN

SEQ ID NO: 25 - protein, 14 aa, synthetic construct
WVRQAPGHGLEWMG

SEQ ID NO: 26 - protein, 17 aa, synthetic construct
WINANTGDPTYAQGFTG

SEQ ID NO: 27 - protein, 32 aa, synthetic construct
RFVFSLDTSVSTAYLQISSLKAEDSAVYYCTR

SEQ ID NO: 28 - protein, 11 aa, synthetic construct
ERFLEWLHFDY

SEQ ID NO: 29 - protein, 11 aa, synthetic construct
WGQGTLVTVSS

SEQ ID NO: 30 - protein, 30 aa, synthetic construct
QVQLVQSGAEVKKPGASVKVSCKVSGYTLT

SEQ ID NO: 31 - protein, 5 aa, synthetic construct
ELSMH

SEQ ID NO: 32 - protein, 14 aa, synthetic construct
WVRQAPGKGLEWMG

SEQ ID NO: 33 - protein, 17 aa, synthetic construct
GFDPEYGKTFFAQNFQG

SEQ ID NO: 34 - protein, 32 aa, synthetic construct
RVTMTEDTSADTAYMELSSLRSEDTAVYYCAT

SEQ ID NO: 35 - protein, 16 aa, synthetic construct
EGYYETTTYYYNLFDS

SEQ ID NO: 36 - protein, 11 aa, synthetic construct
WGQGTLVTVSS

SEQ ID NO: 37 - protein, 30 aa, synthetic construct
QVQLVQSGSELKKPGASVKVSCKTSGYTFT

SEQ ID NO: 38 - protein, 5 aa, synthetic construct
DYAMT

SEQ ID NO: 39 - protein, 14 aa, synthetic construct
WVRQAPGQGLEWMG

SEQ ID NO: 40 - protein, 17 aa, synthetic construct
WITTNTGDPTYAPGFTG

SEQ ID NO: 41 - protein, 32 aa, synthetic construct
RFVFSLDTSVSTAYLQISSLKAEDTAVYYCAR

SEQ ID NO: 42 - protein, 10 aa, synthetic construct
VYHWIRGFEF

SEQ ID NO: 43 - protein, 11 aa, synthetic construct
WGQGTLVTVSS

SEQ ID NO: 44 - protein, 30 aa, synthetic construct
QVQLQESGPGLVKPSETLSLTCTVSGGSFS

SEQ ID NO: 45 - protein, 7 aa, synthetic construct
SSSSYWG

SEQ ID NO: 46 - protein, 14 aa, synthetic construct
WIRQPPGKGLEWIG

SEQ ID NO: 47 - protein, 16 aa, synthetic construct
SFYYSGNTYYNPSLKS

SEQ ID NO: 48 - protein, 32 aa, synthetic construct
RVTISEDTSKNQFSLKLSSVTAADTAVYYCAR

SEQ ID NO: 49 - protein, 16 aa, synthetic construct
QTYSSSWDGVLYYFDY

SEQ ID NO: 50 - protein, 11 aa, synthetic construct
WGQGTLVTVSS

SEQ ID NO: 51 - protein, 30 aa, synthetic construct
QVQLQESGPGLVKPSETLSLTCTVSNGSIS

SEQ ID NO: 52 - protein, 5 aa, synthetic construct
TYYWS

SEQ ID NO: 53 - protein, 14 aa, synthetic construct
WIRQPPGKGLEWIG

SEQ ID NO: 54 - protein, 16 aa, synthetic construct
YVYYTGRTKYNPSLKS

SEQ ID NO: 55 - protein, 32 aa, synthetic construct
RVTISVDTSKNQFSLNLSSVTAADTAVYYCAR

SEQ ID NO: 56 - protein, 18 aa, synthetic construct
GGIVVVPAARDYYYYMDV

SEQ ID NO: 57 - protein, 11 aa, synthetic construct
WGKGTTVTVSS

SEQ ID NO: 58 - protein, 30 aa, synthetic construct
EVQLVQSGAEVKKPGESLKIACKGSGFSFT

SEQ ID NO: 59 - protein, 5 aa, synthetic construct
SHWIG

SEQ ID NO: 60 - protein, 14 aa, synthetic construct
WVRQKPGRGLEWMG

SEQ ID NO: 61 - protein, 17 aa, synthetic construct
VIYPGDSDTRYSPSFQG

SEQ ID NO: 62 - protein, 32 aa, synthetic construct
QVTVSADKSINTAYLQWNSLKASDTAIYYCAR

SEQ ID NO: 63 - protein, 11 aa, synthetic construct
PNSGSPRYFEF

SEQ ID NO: 64 - protein, 11 aa, synthetic construct
WGRGTLVTVSS

SEQ ID NO: 65 - protein, 30 aa, synthetic construct
QVQLQESGPGLVKPSETLSLTCTVSGGSIS

SEQ ID NO: 66 - protein, 7 aa, synthetic construct
SSSYYWG

SEQ ID NO: 67 - protein, 14 aa, synthetic construct
WIRQPPGKGLEWIG

SEQ ID NO: 68 - protein, 16 aa, synthetic construct
SFYYSGNTYYNPSLKS

SEQ ID NO: 69 - protein, 32 aa, synthetic construct
RVTISVDTSKNQFSLKLSSVTAADTAVYYCAR

SEQ ID NO: 70 - protein, 16 aa, synthetic construct
QEYYYGSGSPSYYFDY

SEQ ID NO: 71 - protein, 11 aa, synthetic construct
WGQGTLVTVSS

SEQ ID NO: 72 - protein, 30 aa, synthetic construct
EVQLVQSGAEVKKPGESLKISCKGSGDSFI

SEQ ID NO: 73 - protein, 5 aa, synthetic construct
SHWIA

SEQ ID NO: 74 - protein, 14 aa, synthetic construct
WVRQMPGKGLEWMG

SEQ ID NO: 75 - protein, 17 aa, synthetic construct
IVYPGDSDTRYSPSFQG

SEQ ID NO: 76 - protein, 32 aa, synthetic construct
QVTISADKSITTAYLQWSSLKASDTAMYYCAR

SEQ ID NO: 77 - protein, 11 aa, synthetic construct
HEWELLGPFDY

SEQ ID NO: 78 - protein, 11 aa, synthetic construct
WGQGTLVTVSS

SEQ ID NO: 79 - protein, 30 aa, synthetic construct
EVQLVQSGAEVKKPGSSVKVSCKASGGTST

SEQ ID NO: 80 - protein, 5 aa, synthetic construct
NDAIS

SEQ ID NO: 81 - protein, 14 aa, synthetic construct
WVRQTPGQGLEWMG

SEQ ID NO: 82 - protein, 17 aa, synthetic construct
SIIPILDTTDHAQKFQG

SEQ ID NO: 83 - protein, 32 aa, synthetic construct
RVTITADKSTNTAYMELNSLRSDDTAVYYCAR

SEQ ID NO: 84 - protein, 12 aa, synthetic construct
EHIAARQDYFDY

SEQ ID NO: 85 - protein, 11 aa, synthetic construct
WGQGTLVTVSS

SEQ ID NO: 86 - protein, 30 aa, synthetic construct
EVQLVQSGSKLKKPGASVKVSCKASGYTFT

SEQ ID NO: 87 - protein, 5 aa, synthetic construct
SYTMN

SEQ ID NO: 88 - protein, 14 aa, synthetic construct
WVRQAPGQGLEWMG

SEQ ID NO: 89 - protein, 17 aa, synthetic construct
WINTDTGDPTYAQGFTG

SEQ ID NO: 90 - protein, 32 aa, synthetic construct
RFVFSLDTSVSTAFLQINSLKAEDTAVYYCAR

SEQ ID NO: 91 - protein, 18 aa, synthetic construct
GDCDSTSCYRYSYGYEDY

SEQ ID NO: 92 - protein, 11 aa, synthetic construct
WGQGTLVTVSS

SEQ ID NO: 93 - protein, 30 aa, synthetic construct
QVQLVQSGAEVKKPGSSVKVSCKVSGGTFR

SEQ ID NO: 94 - protein, 5 aa, synthetic construct
SYAIS

SEQ ID NO: 95 - protein, 14 aa, synthetic construct
WVRQAPGQGLEWMG

SEQ ID NO: 96 - protein, 17 aa, synthetic construct
GIIPIFDTRNYAQILQG

SEQ ID NO: 97 - protein, 32 aa, synthetic construct
RVTITADLSTSTAYMELNSLRSEDTAIYYCAR

SEQ ID NO: 98 - protein, 10 aa, synthetic construct
GSDEGDWFDP

SEQ ID NO: 99 - protein, 11 aa, synthetic construct
WGQGTLVTVSS

SEQ ID NO: 100 - protein, 30 aa, synthetic construct
EVQLVQSGTEVRKPGSSVKVSCKASGGTFS

SEQ ID NO: 101 - protein, 5 aa, synthetic construct
NYAIS

SEQ ID NO: 102 - protein, 14 aa, synthetic construct
WVRQAPGQGLEWMG

SEQ ID NO: 103 - protein, 17 aa, synthetic construct
SIIPILGTTDHAQKFQD

SEQ ID NO: 104 - protein, 32 aa, synthetic construct
RVTITADKSSNTTYMELSSLRSDDTAVYYCAR

SEQ ID NO: 105 - protein, 12 aa, synthetic construct
EYIAARLDYFDS

SEQ ID NO: 106 - protein, 11 aa, synthetic construct
WGQGTLVTVSS

SEQ ID NO: 107 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 109 - protein, 107 aa, synthetic construct [sequence omitted]

SEQ ID NO: 110 - protein, 95 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 111 - protein, 6 aa, synthetic construct
QSISSY

SEQ ID NO: 112 - skipped

SEQ ID NO: 113 - protein, 9 aa, synthetic construct
QQSYSTPPT

SEQ ID NO: 114 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 115 - protein, 15 aa, synthetic construct
EPKSCDKTHTCPPCP

SEQ ID NO: 116 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 117 - protein, 106 aa, synthetic construct
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 106 aa, synthetic construct
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 4 aa, synthetic construct
LREA

SEQ ID NO: 120 - protein, 6 aa, synthetic construct
VAIKEL

(19) United States
(12) Patent Application Publication
(10) Pub. No.: US 2026/0295040 A1
(43) Pub. Date: Oct. 1, 2026

(54) TREATMENT OF CANCER WITH A COMBINATION OF AN ANTIBODY THAT BINDS EGFR AND CYTOTOXIC DRUGS

(71) Applicant: Merus N.V., Utrecht (NL)

(72) Inventors: Jeroen Jilles LAMMERTS VAN BUEREN, Utrecht (NL); Mark THROSBY, Utrecht (NL)

(21) Appl. No.: 19/144,095

(22) PCT Filed: Dec. 28, 2023
(86) PCT No.: PCT/NL2023/050692

§ 371 Date: Jun. 27, 2025

(30) Foreign Application Priority Data
Dec. 28, 2022 (NL) 2033862

(51) Int. Cl. A61K 39/395; A61K 9/00; A61K 31/282; A61K 31/4745; A61K 31/513; A61K 31/519; A61K 31/635; A61K 31/7072; A61P 35/00; C07K 16/28

(52) U.S. Cl. CPC A61K 39/3955; A61K 9/0019; A61K 31/282; A61K 31/4745; A61K 31/513; A61K 31/519; A61K 31/635; A61K 31/7072; A61P 35/00; C07K 16/28; C07K 16/2863; C07K 2317/31

(57) ABSTRACT

The present disclosure relates to the field of therapeutic antibodies for the treatment of a subject with cancer. More in particular, it relates to treating cancer using an antibody or functional part, derivative and/or analogue thereof that comprises a variable domain that binds an extracellular part of EGFR in a combination treatment that further comprises administering a fluoropyrimidine, a platinum-based chemotherapeutic agent, a BCL-2 inhibitor or SN-38. The cancers can be head and neck cancer, gastric cancer, esophageal cancer, gastro-esophageal-junction cancer, non-small cell lung cancer or colorectal cancer. The disclosure also relates to pharmaceutical formulations, kit-of-parts and dosage regimes.

### FIELD OF THE DISCLOSURE

The present disclosure relates to means and methods in the treatment of cancer. The present disclosure in particular relates to a method of treating cancer in a subject using a combination of an antibody that binds EGFR and a fluoropyrimidine, a platinum-based chemotherapeutic agent, a BCL-2 inhibitor, or SN-38. The present disclosure further relates to the combination for use in such methods and to the combination for use in the manufacture of a medicament for the treatment of cancer.

### BACKGROUND OF THE DISCLOSURE

Colorectal cancer (CRC) is a leading cause of death worldwide, one of the reasons being its increasing resistance to current treatments. While some new treatments have been advanced in CRC, many have failed clinical testing and metastatic CRC is still largely incurable. The current standard-of-care for advanced CRC in the clinics includes chemotherapy regimens which block essential functions in cancer cells and kill dividing cells.

Several anti-EGFR drugs have demonstrated certain levels of efficacy for targeted therapy of metastatic CRC (mCRC). However, due to various reasons, resistance to anti-EGFR therapies is observed, for instance, due to emergence of mutations conferring resistance or the presence of innate resistance to anti-EGFR therapies. Most of the patients in which the cancers are sensitive to anti-EGFR treatment, develop resistant cancer later on.

Current standard of care treatments for CRC include oxaliplatin, fluorouracil and irinotecan. In case of liver metastases, the standard of care treatments are combined either with EGFR or VEGF blocking monoclonal antibodies.

Due to increasing resistance to current treatments, there remains a need to develop further therapies targeting cancers such as CRC, to improve cancer treatments or broaden treatment options.

### SUMMARY OF THE INVENTION

The disclosure provides an antibody or functional part, derivative and/or analogue thereof that comprises a variable domain that binds an extracellular part of EGFR, for use in the treatment of a cancer in a subject, wherein the treatment further comprises administering of a fluoropyrimidine, a platinum-based chemotherapeutic agent, a BCL-2 inhibitor, or SN-38. In certain aspects, the disclosure provides an antibody or functional part, derivative and/or analogue thereof that comprises a variable domain that binds an extracellular part of EGFR, for use in the treatment of a cancer in a subject, wherein the treatment further comprises administering of a fluorouracil, TAS-102, oxaliplatin, venetoclax, or SN-38.

The present disclosure shows that a combination therapy comprising the administration of a multispecific antibody that targets EGFR and LGR5, in combination with a fluoropyrimidine, a platinum-based chemotherapeutic agent, a BCL-2 inhibitor, or SN-38, is more effective when compared to the effect of the multispecific antibody or the fluoropyrimidine, the platinum-based chemotherapeutic agent, the BCL-2 inhibitor, or SN-38 administered separately.

In certain aspects, the disclosure further provides a combination of an antibody or functional part, derivative and/or analogue thereof and a fluoropyrimidine, a platinum-based chemotherapeutic agent, a BCL-2 inhibitor, or SN-38, for use in the treatment of cancer.

In certain aspects, the disclosure provides an antibody or functional part, derivative and/or analogue thereof that comprises a variable domain that binds an extracellular part of EGFR, for use in the treatment of a cancer in a subject, wherein the treatment further comprises administering fluorouracil, oxaliplatin and folinic acid.

In certain aspects, the disclosure provides an antibody or functional part, derivative and/or analogue thereof that comprises a variable domain that binds an extracellular part of EGFR, for use in the treatment of a cancer in a subject, wherein the treatment further comprises administering fluorouracil, irinotecan and folinic acid.

Further provided are methods of treatment of cancer in a subject comprising administering an effective amount of an antibody or functional part, derivative and/or analogue thereof that comprises a variable domain that binds an extracellular part of EGFR, and fluorouracil, TAS-102, oxaliplatin, venetoclax, or SN-38, to the subject. In certain aspects, said fluorouracil, TAS-102, oxaliplatin, venetoclax, or SN-38 are administered in an effective amount to said subject.

Further provided are combination therapies, wherein a subject having cancer is administered a combination therapy, which combination comprises an effective amount of an antibody or functional part, derivative and/or analogue thereof that comprises a variable domain that binds an extracellular part of EGFR, and fluorouracil, TAS-102, oxaliplatin, venetoclax, or SN-38 to the subject having cancer.

In certain aspects, said antibody or functional part, derivative and/or analogue thereof that comprises a variable domain that binds an extracellular part of EGFR, is administered with fluorouracil, folinic acid, and irinotecan (FOLFIRI) or folinic acid, fluorouracil, and oxaliplatin (FOLFOX). In certain aspects, said subject has received prior treatment for said cancer. In certain aspects, said subject is administered said chemotherapy as second-line treatment. In certain aspects, said subject is administered said chemotherapy as third line or later treatment. In certain aspects, said subject suffers from colorectal cancer, in particular metastatic colorectal cancer (mCRC). In certain aspects, treatment of the present disclosure further comprises administering to said subject petosemtamab at 1500 mg every two weeks (Q2W). In certain aspects, said subject suffers from mCRC and is wildtype for RAS and/or RAF (including Kirsten Rat Sarcoma (KRAS) and/or B-Rapidly Accelerated Fibrosarcoma (BRAF)). In certain aspects, the genome of said mCRC is wildtype for RAS and/or RAF, including KRAS and/or BRAF.

In certain aspects, the cancer is an adenocarcinoma or a squamous cell carcinoma. In certain aspects, the cancer is a head and neck cancer, gastric, esophageal, gastro-esophageal-junction cancer, non-small cell lung cancer or colorectal cancer. In certain aspects, the cancer is colorectal cancer.

In certain aspects, the subject of the present disclosure is a mammalian subject, such as a human subject.

In certain aspects, the subject has progressed after prior anti-cancer therapy, wherein the anti-cancer therapy is chemotherapy, targeted therapy, immunotherapy or radiation therapy.

In certain aspects, the antibody or functional part, derivative and/or analogue thereof is ADCC enhanced. Also, in certain aspects, the antibody or functional part, derivative and/or analogue thereof is afucosylated.

In certain aspects, the antibody or functional part, derivative and/or analogue thereof of the present disclosure is a multispecific antibody. In certain aspects, said antibody or functional part, derivative and/or analogue thereof is a bispecific antibody. In certain aspects, said antibody or functional part, derivative and/or analogue thereof is a bispecific antibody that at least binds EGFR. In certain aspects, said antibody or functional part, derivative and/or analogue thereof comprises a second variable domain that does not bind EGFR. In certain aspects, said antibody or functional part, derivative and/or analogue thereof binds EGFR monovalently. In certain aspects, said antibody or functional part, derivative and/or analogue thereof comprises a second variable domain that binds LGR5.

In certain aspects, the subject to be treated has not received prior anti-cancer therapy. In certain aspects, the subject to be treated has not received prior chemotherapy, targeted therapy, immunotherapy or radiation therapy. In certain aspects, the subject to be treated has not received prior therapy with fluorouracil, oxaliplatin, FOLFOX, FOLFIRI, TAS-102, trastuzumab, pembrolizumab, nivolumab, cetuximab or a combination thereof.

In certain aspects, the antibody or functional part, derivative and/or analogue thereof is administered to the subject simultaneously, separately or sequentially with the fluoropyrimidine, platinum-based chemotherapeutic agent, BCL-2 inhibitor, or SN-38.

In certain aspects, the disclosure provides a pharmaceutical composition comprising an antibody or functional part, derivative and/or analogue thereof and a fluoropyrimidine, a platinum-based chemotherapeutic agent, a BCL-2 inhibitor, or SN-38.

In certain aspects, the disclosure further provides a kit of parts, comprising an antibody or functional part, derivative and/or analogue thereof and a fluoropyrimidine, a platinum-based chemotherapeutic agent, a BCL-2 inhibitor, or SN-38 and instructions for use of said antibody or functional part, derivative and/or analogue thereof and instructions for use of said fluoropyrimidine, platinum-based chemotherapeutic agent, BCL-2 inhibitor, or SN-38.

In certain aspects, the disclosure provides the use of an antibody or functional part, derivative and/or analogue thereof and a fluoropyrimidine, a platinum-based chemotherapeutic agent, a BCL-2 inhibitor, or SN-38, in the manufacture of a medicament for the treatment of a cancer in a subject, wherein the treatment comprises administering the antibody or functional part, derivative and/or analogue thereof and the fluoropyrimidine, the platinum-based chemotherapeutic agent, the BCL-2 inhibitor, or SN-38, simultaneously, separately, or sequentially.

In certain aspects, the present disclosure relates to a use of an antibody or functional part, derivative and/or analogue thereof that comprises a variable domain that binds an extracellular part of EGFR, and optionally comprises a variable domain that binds an extracellular part of LGR5, and a fluoropyrimidine, a platinum-based chemotherapeutic agent, a BCL-2 inhibitor, or SN-38 in the manufacture of one or more medicaments for treating a cancer in a subject. In certain aspects, the antibody or functional part, derivative and/or analogue thereof and the fluoropyrimidine, platinum-based chemotherapeutic agent, BCL-2 inhibitor, or SN-38 are used to manufacture separate medicaments, such as two separate medicaments, one for said antibody or functional part, derivative and/or analogue thereof and one for said fluoropyrimidine, platinum-based chemotherapeutic agent, BCL-2 inhibitor, or SN-38. Said medicament comprising said antibody, functional part, derivative and/or analogue thereof that comprises a variable domain that binds an extracellular part of EGFR may be contained in a holder that is separate, meaning not physically linked, from a holder that contains said fluoropyrimidine, platinum-based chemotherapeutic agent, BCL-2 inhibitor, or SN-38 as a medicament.

In certain aspects, said antibody or functional part, derivative and/or analogue thereof that comprises a variable domain that binds an extracellular part of EGFR, and optionally comprises a variable domain that binds an extracellular part of LGR5, is or comprises petosemtamab.

In certain aspects, the present disclosure provides the use of an antibody or functional part, derivative and/or analogue of the present disclosure that comprises a variable domain that can bind an extracellular part of EGFR in the manufacture of a medicament for increasing the effect of a fluoropyrimidine, a platinum-based chemotherapeutic agent, a BCL-2 inhibitor, or SN-38 for the treatment of cancer.

In certain aspects, the present disclosure provides the use of a fluoropyrimidine, a platinum-based chemotherapeutic agent, a BCL-2 inhibitor, or SN-38 in the manufacture of a medicament for increasing the effect of an antibody or functional part, derivative and/or analogue of the present disclosure that comprises a variable domain that can bind an extracellular part of EGFR for the treatment of cancer.

Also, the present disclosure provides a combination of an antibody or functional part, derivative and/or analogue thereof that comprises a variable domain that can bind an extracellular part of EGFR and a fluoropyrimidine, a platinum-based chemotherapeutic agent, a BCL-2 inhibitor, or SN-38 as mentioned herein for use in the treatment of cancer in a subject in need thereof.

Also, the present disclosure provides a combination of a fluoropyrimidine, a platinum-based chemotherapeutic agent, a BCL-2 inhibitor, or SN-38, instructions for use of said fluoropyrimidine, platinum-based chemotherapeutic agent, BCL-2 inhibitor, or SN-38, in the treatment of cancer in a subject, as well as instructions for use in the treatment of said cancer in a subject of an antibody or functional part, derivative and/or analogue thereof that comprises a variable domain that can bind an extracellular part of EGFR.

Also, the present disclosure provides a combination of an antibody or functional part, derivative and/or analogue thereof that comprises a variable domain that can bind an extracellular part of EGFR, instructions for use of said antibody or functional part, derivative and/or analogue thereof in the treatment of cancer in a subject, as well as instructions for use in the treatment of said cancer in a subject of a fluoropyrimidine, a platinum-based chemotherapeutic agent, a BCL-2 inhibitor, or SN-38.

In certain aspects, the present disclosure provides a pharmaceutical composition comprising an antibody or functional part, derivative and/or analogue thereof that comprises a variable domain that can bind an extracellular part of EGFR of the present disclosure and instructions for use thereof with a fluoropyrimidine, a platinum-based chemotherapeutic agent, a BCL-2 inhibitor, or SN-38 in the treatment of a cancer.

In certain aspects, the present disclosure provides a pharmaceutical composition for the treatment of a cancer, comprising an antibody or functional part, derivative and/or analogue thereof that comprises a variable domain that can bind an extracellular part of EGFR of the present disclosure and a pharmaceutical composition for the treatment of said cancer, comprising a fluoropyrimidine, a platinum-based chemotherapeutic agent, a BCL-2 inhibitor, or SN-38.

In certain aspects, the present disclosure provides a pharmaceutical composition for use in the treatment of cancer comprising an antibody or functional part, derivative and/or analogue thereof that comprises a variable domain that can bind an extracellular part of EGFR of the present disclosure, wherein the pharmaceutical composition is administered in combination with a fluoropyrimidine, a platinum-based chemotherapeutic agent, a BCL-2 inhibitor, or SN-38 of the present disclosure.

In certain aspects, the present disclosure relates to a pharmaceutical composition for the treatment of a cancer comprising an antibody or functional part, derivative and/or analogue thereof that comprises a variable domain that can bind an extracellular part of EGFR of the present disclosure, wherein a subject to be treated in the treatment is administered a fluoropyrimidine, a platinum-based chemotherapeutic agent, a BCL-2 inhibitor, or SN-38 prior to, simultaneously with, or after administration of said antibody.

In certain aspects, the present disclosure relates to a pharmaceutical composition for the treatment of a cancer in a subject comprising a fluoropyrimidine, a platinum-based chemotherapeutic agent, a BCL-2 inhibitor, or SN-38, wherein said subject to be treated is administered an antibody or functional part, derivative and/or analogue thereof that comprises a variable domain that can bind an extracellular part of EGFR of the present disclosure prior to, simultaneously with, or after administration of said fluoropyrimidine, platinum-based chemotherapeutic agent, BCL-2 inhibitor, or SN-38.

The present disclosure thus relates to a combination of medicaments for the treatment of cancer in a subject which comprises administration to said subject of multiple, different medicaments for treating said cancer, which treatment comprises simultaneous, sequential or separate administration of said medicaments. In certain aspects, said medicament comprises an antibody or functional part, derivative and/or analogue thereof that comprises a variable domain that can bind an extracellular part of EGFR of the present disclosure, and said other, different medicament comprises a fluoropyrimidine, a platinum-based chemotherapeutic agent, a BCL-2 inhibitor, or SN-38.

In certain aspects, the antibody or functional part, derivative and/or analogue thereof that comprises a variable domain that can bind an extracellular part of EGFR of the present disclosure may be administered simultaneously, sequentially or separately with the fluoropyrimidine, platinum-based chemotherapeutic agent, BCL-2 inhibitor, or SN-38 of the present disclosure. Said combination of the antibody or functional part, derivative and/or analogue thereof that comprises a variable domain that can bind an extracellular part of EGFR of the present disclosure and fluoropyrimidine, platinum-based chemotherapeutic agent, BCL-2 inhibitor, or SN-38 thus encompasses simultaneous, sequential or separate administration.

Hence, in certain aspects, the present disclosure provides an antibody or functional part, derivative and/or analogue thereof that comprises a variable domain that can bind an extracellular part of EGFR of the present disclosure for use in a method of treatment of a cancer, wherein the treatment further comprises administering of a fluoropyrimidine, a platinum-based chemotherapeutic agent, a BCL-2 inhibitor, or SN-38, wherein optionally said antibody or functional part, derivative and/or analogue thereof is administered simultaneously, sequentially or separately with said fluoropyrimidine, platinum-based chemotherapeutic agent, BCL-2 inhibitor, or SN-38.

Hence, in certain aspects, the present disclosure provides a method of treatment of a subject having a cancer, comprising administering to the subject an effective amount of a fluoropyrimidine, a platinum-based chemotherapeutic agent, a BCL-2 inhibitor, or SN-38 and an antibody or functional part, derivative and/or analogue thereof that comprises a variable domain that can bind an extracellular part of EGFR of the present disclosure, wherein optionally said antibody or functional part, derivative and/or analogue thereof is administered simultaneously, sequentially or separately with said fluoropyrimidine, platinum-based chemotherapeutic agent, BCL-2 inhibitor, or SN-38.

Hence, in certain aspects, the present disclosure provides the use of an antibody or functional part, derivative and/or analogue thereof that comprises a variable domain that can bind an extracellular part of EGFR of the present disclosure and a fluoropyrimidine, a platinum-based chemotherapeutic agent, a BCL-2 inhibitor, or SN-38 in the manufacture of a medicament for the treatment of a cancer, wherein optionally said antibody or functional part, derivative and/or analogue thereof is administered simultaneously, sequentially or separately with said fluoropyrimidine, platinum-based chemotherapeutic agent, BCL-2 inhibitor, or SN-38. In certain aspects, said antibody or functional part, derivative and/or analogue thereof is administered prior to, simultaneously with, or after administration of said fluoropyrimidine, platinum-based chemotherapeutic agent, BCL-2 inhibitor, or SN-38.

In certain aspects, the antibody or functional part, derivative and/or analogue thereof that comprises a variable domain that can bind an extracellular part of EGFR of the present disclosure is for use in the manufacture of a medicament for the treatment of a cancer and the fluoropyrimidine, platinum-based chemotherapeutic agent, BCL-2 inhibitor, or SN-38 are for use in the manufacture of a medicament for the treatment of said cancer, wherein optionally said antibody or functional part, derivative and/or analogue thereof is administered simultaneously, sequentially or separately with said fluoropyrimidine, platinum-based chemotherapeutic agent, BCL-2 inhibitor, or SN-38. Optionally, said antibody or functional part, derivative and/or analogue thereof is administered prior to, simultaneously with, or after administration of said fluoropyrimidine, platinum-based chemotherapeutic agent, BCL-2 inhibitor, or SN-38.

In certain aspects, the antibody or functional part, derivative and/or analogue thereof that comprises a variable domain that can bind an extracellular part of EGFR of the present disclosure is for use in the manufacture of a medicament for the treatment of a cancer, whereby said treatment is combined with FOLFIRI, wherein optionally said antibody or functional part, derivative and/or analogue thereof is administered simultaneously, sequentially or separately with said FOLFIRI. Optionally, said antibody or functional part, derivative and/or analogue thereof is administered prior to, simultaneously with, or after administration of said FOLFIRI.

In certain aspects, the antibody or functional part, derivative and/or analogue thereof that comprises a variable domain that can bind an extracellular part of EGFR of the present disclosure is for use in the manufacture of a medicament for the treatment of a cancer, whereby said treatment is combined with FOLFOX, wherein optionally said antibody or functional part, derivative and/or analogue thereof is administered simultaneously, sequentially or separately with said FOLFOX. Optionally, said antibody or functional part, derivative and/or analogue thereof is administered prior to, simultaneously with, or after administration of said FOLFOX.

### BRIEF DESCRIPTION OF THE DRAWINGS

FIG. 1: Human LGR5 sequence; Sequence ID NO: 1.

FIG. 2: Human EGFR sequence; Sequence ID NO: 2.

FIG. 3: (a) Amino acid sequences of heavy chain variable regions (Sequence ID Nos: 3-15) that together with a common light chain variable region such as the variable region of the human kappa light chain IGKV1-39/jk1 (SEQ ID NO: 107), form a variable domain that binds LGR5 and EGFR. The heavy chain CDRs (according to KABAT numbering system) and framework regions are indicated in FIG. 3b. FIG. 4: a) Amino acid sequence of a common light chain. b) Common light chain variable region (IgVk1-39/jk1). c) Light chain constant region. d) V-region IgVk1-39; e) CDR1, CDR2 and CDR3 of a common light chain according to IMGT numbering.

FIG. 5: IgG heavy chains for the generation of bispecific molecules. a) CH1 region DNA. b) Hinge region. c) CH2 region. d) CH3 domain containing variations L351K and T366K (KK). e) CH3 domain containing variations L351D and L368E (DE). Residue positions are according to EU numbering.

### DETAILED DESCRIPTION

In certain aspects, the present disclosure provides an antibody or functional part, derivative and/or analogue thereof that comprises a variable domain that binds an extracellular part of EGFR, for use in the treatment of a cancer in a subject, wherein the treatment further comprises administering of a fluoropyrimidine, a platinum-based chemotherapeutic agent, a BCL-2 inhibitor, or SN-38.

In certain aspects, the present disclosure provides a method of treating cancer in a subject, the method comprising administering an effective amount of an antibody or functional part, derivative and/or analogue thereof that comprises a variable domain that binds an extracellular part of EGFR, and a fluoropyrimidine, a platinum-based chemotherapeutic agent, a BCL-2 inhibitor, or SN-38, to the subject.

In certain aspects, the present disclosure provides an antibody or functional part, derivative and/or analogue thereof that comprises a variable domain that binds an extracellular part of EGFR and a variable domain that binds an extracellular part of LGR5, for use in the treatment of a cancer in a subject, wherein the treatment further comprises administering of a fluoropyrimidine, a platinum-based chemotherapeutic agent, a BCL-2 inhibitor, or SN-38.

In certain aspects, the present disclosure provides a method of treating cancer in a subject, the method comprising administering an effective amount of an antibody or functional part, derivative and/or analogue thereof that comprises a variable domain that binds an extracellular part of EGFR and a variable domain that binds an extracellular part of LGR5, and a fluoropyrimidine, a platinum-based chemotherapeutic agent, a BCL-2 inhibitor, or SN-38, to the subject.

### Cancer Types

In certain aspects, the cancer of the present disclosure is an adenocarcinoma or a squamous cell carcinoma.

In certain aspects, the cancer is selected from gastric cancer, esophageal cancer, gastric-esophageal-junction cancer, head and neck cancer, non-small cell lung cancer or colorectal cancer, in particular squamous cell carcinoma of the head and neck (SCCHN).

In certain aspects, the cancer is gastric, esophageal, or gastro-esophageal-junction cancer. Gastric cancer (also referred to as stomach cancer) is a cancer that develops from the lining of the stomach and in particular the mucus-producing glandular cells found therein. Such a cancer is also referred to as adenocarcinoma, or in this case gastric adenocarcinoma develops from the lining of the stomach. In particular, the cancer is thus a gastric adenocarcinoma or cancer that develops from the lining of the stomach which is used interchangeably herein. Esophageal cancer is a cancer that develops from the esophagus. The two main subtypes are ESCC (esophageal squamous-cell carcinoma) and EAC (esophageal adenocarcinoma). Gastro-esophageal junction cancer (also known as gastro-esophageal junction adenocarcinoma) arises from the gastro-esophageal junction.

Cancers that are known collectively as head and neck cancers usually originate in the squamous cells that line the moist, mucosal surfaces inside the head and neck, such as inside the mouth, the nose, and the throat. These squamous cell cancers are often referred to as squamous cell carcinomas of the head and neck and said cancers are treated in certain aspects of the present disclosure. Although rare, head and neck cancers can also occur in the salivary glands. In particular, the head and neck cancer may occur in the oral cavity. This includes the lips, the front two-thirds of the tongue, the gums, the lining inside the cheeks and lips, the floor of the mouth under the tongue, the hard palate, and the small area of the gum behind the wisdom teeth.

Thus, in certain aspects, the head and neck cancer is squamous cell carcinoma and includes nasopharyngeal cancer, laryngeal cancer, hypopharyngeal cancer, nasal cavity cancer, paranasal sinus cancer, oral cancer, oropharyngeal cancer or salivary gland cancer. More in particular, the present disclosure relates to treatment of a cancer comprising a squamous cell head and neck cancer, such as located in the oropharynx, hypopharynx, the larynx, the oral cavity or the tongue. Also, the head and neck cancer is in particular a squamous cell carcinoma of unknown primary (also referred to in the art as a cancer of unknown primary or CUP).

In certain aspects, the cancer is a non-small cell lung cancer.

In certain aspects the cancer is a colorectal cancer. In certain aspects, said cancer is metastatic colorectal cancer (mCRC). In certain aspects, said subject for treatment has been diagnosed with adenocarcinoma of the colon or rectum. In certain aspects, said cancer has been previously treated, such as with first line standard of care as appropriate for CRC or mCRC. In certain aspects, said subject or said cancer has been treated and receives second line treatment using an antibody or functional part, derivative and/or analogue thereof that comprises a variable domain that binds an extracellular part of EGFR and optionally a variable domain that binds an extracellular part of LGR5, in combination with either a fluoropyrimidine, a platinum-based chemotherapeutic agent, a BCL-2 inhibitor, or SN-38.

In certain aspects, said subject or cancer has been treated and is treated using an antibody or functional part, derivative and/or analogue thereof that comprises a variable domain that binds an extracellular part of EGFR and optionally a variable domain that binds an extracellular part of LGR5, in combination with either a fluoropyrimidine, a platinum-based chemotherapeutic agent, a BCL-2 inhibitor, or SN-38 as third line treatment or later. In certain aspects, said prior treatment comprises standard of care treatment appropriate for CRC or mCRC. In certain aspects, said standard of care comprises or is chemotherapy appropriate for CRC or mCRC. In certain aspects, said prior treatment comprises or is fluoropyrimidine-oxaliplatin-based chemotherapy with or without bevacizumab, or fluoropyrimidine-irinotecan-based chemotherapy with or without bevacizumab appropriate for CRC or mCRC.

In certain aspects, said subject to be treated suffers from CRC and is wildtype for RAS. In certain aspects, said subject to be treated suffers from CRC and is wildtype for RAF. RAS includes Kirsten Rat Sarcoma (KRAS), RAF includes B-Rapidly Accelerated Fibrosarcoma (BRAF)). In certain aspects, the genome of said CRC is wildtype for RAS and/or RAF, including KRAS and/or BRAF. In certain aspects, the genome of a sample obtained from said CRC is wildtype for RAS and/or RAF, including KRAS and/or BRAF.

In certain aspects, said subject to be treated suffers from mCRC and is wildtype for RAS. In certain aspects, said subject to be treated suffers from mCRC and is wildtype for RAF. RAS includes Kirsten Rat Sarcoma (KRAS), RAF includes B-Rapidly Accelerated Fibrosarcoma (BRAF)). In certain aspects, the genome of said mCRC is wildtype for RAS and/or RAF, including KRAS and/or BRAF. In certain aspects, the genome of a sample obtained from said mCRC is wildtype for RAS and/or RAF, including KRAS and/or BRAF.

In certain aspects, said subject has been previously diagnosed with histologically or cytologically confirmed unresectable or metastatic adenocarcinoma of the colon or rectum. In certain aspects, said subject is RAS and/or RAF wildtype as determined using Next-Generation Sequencing (NGS) on tumor tissue (primary or metastatic). In certain aspects, said subject has not received prior therapy with anti-EGFR therapy. In other words, said subject is naive to prior anti-EGFR therapy. In certain aspects, said subject has radiographically confirmed disease progression which occurred during or within six months following said prior (first line) chemotherapy.

In certain aspects, the cancer is a cancer that expresses EGFR. In certain aspects, the cancer is a cancer that expresses LGR5. In certain aspects, the cancer is a cancer that expresses EGFR and LGR5.

In certain aspects, the cancer is gastric cancer that expresses EGFR. In certain aspects, the cancer is esophageal cancer that expresses EGFR. In certain aspects, the cancer is gastric-esophageal-junction cancer that expresses EGFR. In certain aspects, the cancer is head and neck cancer that expresses EGFR. In certain aspects, the cancer is non-small cell lung cancer that expresses EGFR. In certain aspects, the cancer is colorectal cancer that expresses EGFR. In certain aspects, the cancer is squamous cell carcinoma of the head and neck (SCCHN) that expresses EGFR.

As used herein, a cancer expresses EGFR if the cancer comprises cells that express EGFR. A cell which expresses EGFR comprises detectable levels of RNA that codes for EGFR. In certain aspects, EGFR expression is determined by ISH. As used herein, a cancer expresses LGR5 if the cancer comprises cells that express LGR5. A cell which expresses LGR5 comprises detectable levels of RNA that codes for LGR5.

In certain aspects, EGFR protein expression is detected by IHC. In certain aspects, EGFR expression is determined by IHC using a commercially available EGFR detection kit, such as the EGFR pharmDx™ kit for a Dako autostainer (Agilent), using the manufacturer's recommendations or the commercially available IHC EGFR detection kit based on EGFR clone 113 which binds the EGFR extracellular domain (Leica, https://shop.leicabiosystems.com/us/ihc-ish/ihc-primary-antibodies/pid-epidermal-growth-factor-receptor). Alternatively, EGFR expression is determined using the Novocastra™ Liquid Mouse Monoclonal Antibody Epidermal Growth Factor Receptor which is based on clone EGFR.113 (Product Code: NCL-L-EGFR, Epidermal Growth Factor Receptor —IHC Primary Antibodies by leicabiosystems.com).

Briefly, the commercially available EGFR pharmDx™ IHC kit system contains reagents required to complete an IHC staining procedure for routinely-fixed, paraffin-embedded specimens. Following incubation with the primary, non-Her2, Her3 and Her4 cross-reactive, monoclonal antibody (clone 2-18C9) to human EGFR protein, this kit employs a ready-to-use visualization reagent based on dextran technology. This reagent consists of both secondary goat anti-mouse antibody molecules and horseradish peroxidase molecules linked to a common dextran polymer backbone. The enzymatic conversion of the subsequently added chromogen results in formation of a visible reaction product at the antigen site. Results are routinely assessed using a light microscope. Control slides containing two formalin-fixed, paraffin-embedded human cell lines with staining intensity scores of 2+ and 0 are provided for quality control of the kit reagent performance.

Staining intensity is established as follows: 3+(strong staining): visible at low levels of magnification, ×5 objective lens which could be confirmed at higher levels as required; 2+(moderate staining): visible at intermediate levels of magnification, ×10 or ×20 objective lenses; 1+(weak staining): only reliably confirmable at high magnification, ×40 objective lens; 0 (no staining): no staining visible at high magnification.

In certain aspects, EGFR expression is determined using immunohistochemistry (IHC) and the cancer is IHC positive for EGFR. In certain aspects, the cancer is characterized by an EGFR IHC score of 2+ or 3+. In certain aspects, the cancer is characterized by an H-score for EGFR of more than 50, more than 80 or more than 200, but not more than 300.

In certain aspects, EGFR expression is determined using immunohistochemistry (IHC) followed by assigning an H-score for EGFR using a range of 0-300. In certain aspects, the cancer of the present disclosure is a cancer characterized by an H-score for EGFR of more than 200 on a scale of 0-300. In certain aspects, the EGFR H score is thus between 200 and 300. In certain aspects, the cancer of the present disclosure is a cancer characterized by an H-score for EGFR of more than 50 on a scale of 0-300. In certain aspects, the EGFR H score is thus between 50 and 300. In certain aspects, the cancer of the present disclosure is a cancer characterized by an H-score for EGFR of more than 80 on a scale of 0-300. In certain aspects, the EGFR H score is thus between 80 and 300.

Herein, determining the H-score to assign the EGFR expression status involves a first step of establishing intensity of membrane staining (resulting in a scoring of 0, 1+, 2+, or 3+) which is determined for each cell in a predefined field as described herein. Subsequently, the percentage of cells at each staining intensity level is calculated, and finally, an H-score is assigned using the following formula: [1×(% cells having 1+staining)+2×(% cells having 2+staining)+3×(% cells having 3+staining)]resulting in an H-score for EGFR between 0-300. As a result, the H-score gives more relative weight to higher intensity or amount of staining in a given tumor sample.

Optionally, the treatment with the antibody or functional part, derivative and/or analogue thereof comprises (or in certain aspects is preceded by) a step of diagnosing the subject for EGFR status. In certain aspects, subjects having an IHC score of 3+, or said cancer characterized by an H-score for EGFR of more than 200, on a scale of 0-300, are selected for treatment. In certain aspects, the treatment of a subject is preceded by a step of diagnosing said subject of having an H-score for EGFR of more than 50 on a scale of 0-300. In certain aspects, the treatment of a subject is preceded by a step of diagnosing said subject of having an H-score for EGFR of more than 80 on a scale of 0-300. In certain aspects, the treatment of a subject is preceded by a step of diagnosing said subject of having an H-score for EGFR of more than 200 on a scale of 0-300. After diagnosing, a subject having a score falling within the selected range (i.e. 50-300, 80-300 or 200-300) is selected for treatment according to the present disclosure.

In certain aspects, the cancer expresses LGR5. As used herein, a cancer expresses LGR5 if the cancer comprises cells that express LGR5. A cell which expresses LGR5 comprises detectable levels of RNA that codes for LGR5.

Expression can often also be detected by incubating the cell with an antibody that binds to LGR5. However, some cells do not express the protein high enough for such an antibody test. In such cases mRNA or other forms of nucleic acid sequence detection is preferred. In certain aspects, LGR5 is detected via mRNA expression. In certain aspects, LGR5 detection is by RNA sequencing. In certain aspects, LGR5 detection is by Tissue MicroArray (TMA) staining. In certain aspects, LGR5 expression is determined using In-Situ Hybridization (ISH). Thus preferably, the cancer is ISH positive for LGR5. ISH positive preferably means that expression is characterized by an H-score of 1 or more.

Techniques for detection and scoring on the basis of TMA, ISH and IHC are each well known in the art to the person of ordinary skill and typically commercially available as a standard kit. Quantifying mRNA levels using ISH and expressing such on the basis of an H-score, such as for LGR5, can be performed using commercially available kits, such as the RNAscope® kit from Advanced Cell Diagnostics (Hayward, CA, USA) on a staining platform such as the BondRx platform (Leica, Wetzlar, Germany). Typically, the ISH H-score for LGR5 quantification ranges from 0-400. Alternatively, LGR5 expression is determined by RNA sequencing (RNAseq).

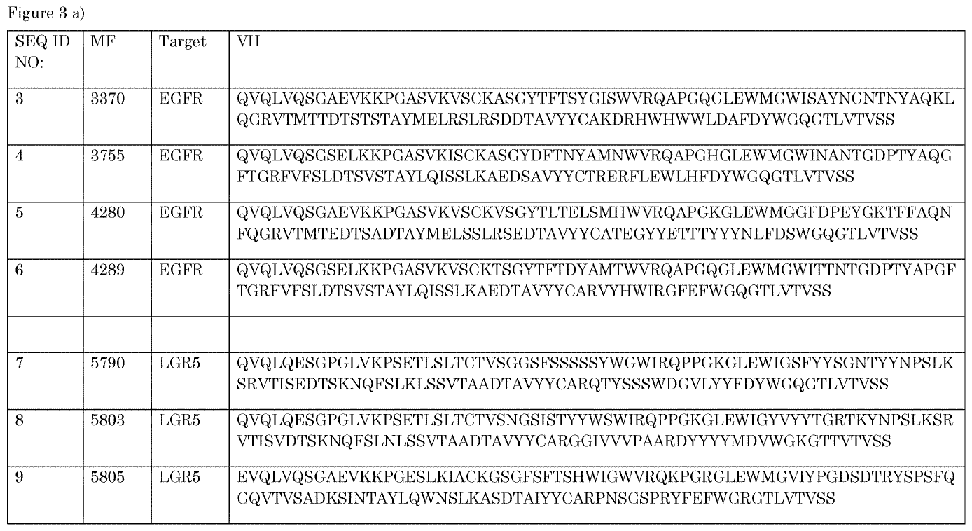

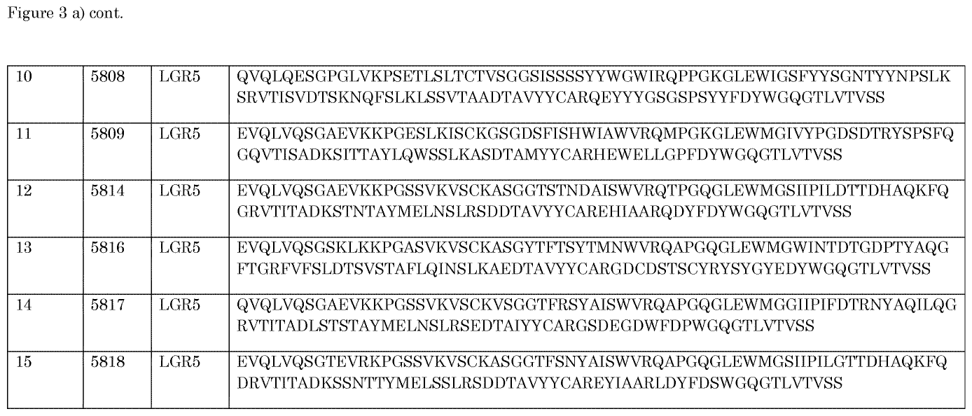

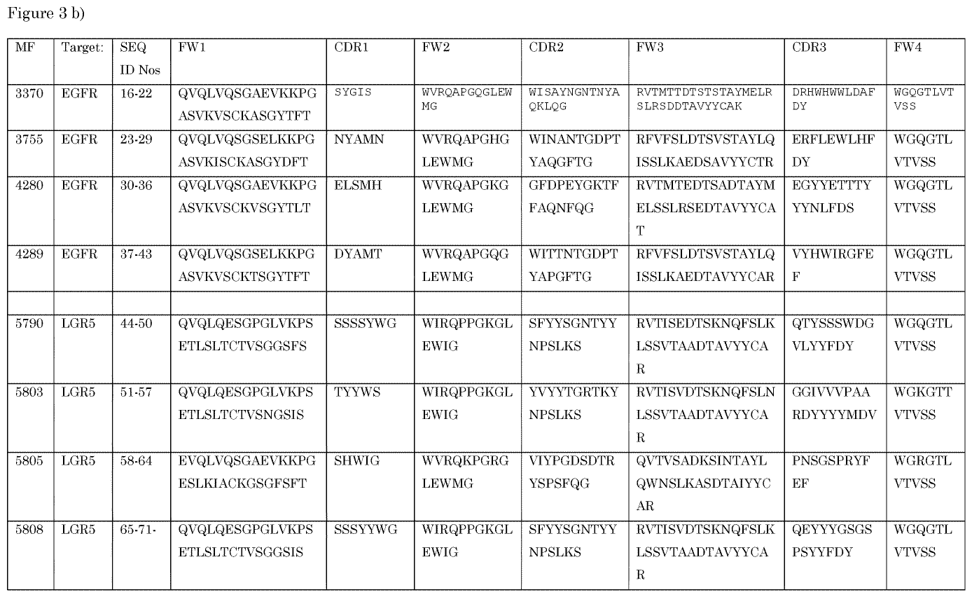

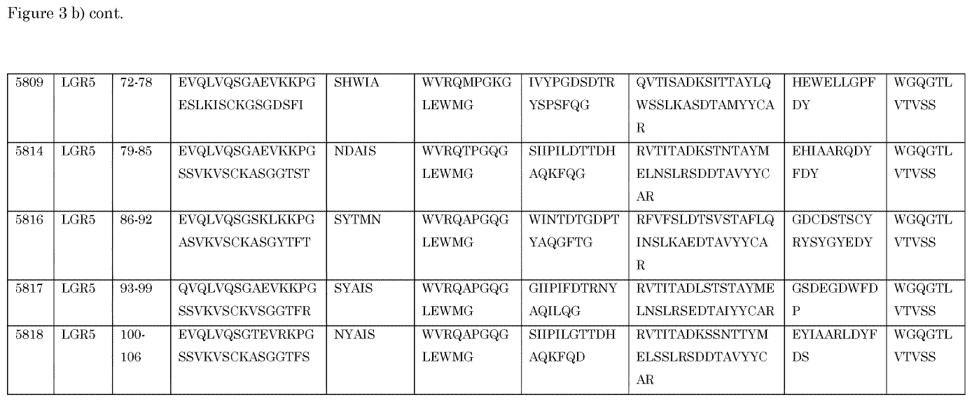

In certain aspects, the cancer expresses LGR5 in sufficient levels for an antibody to bind the LGR5 protein, such as an antibody comprising a VH chain of the variable domain that binds LGR5 that comprises the amino acid sequence of the VH chain of MF5816 as depicted in FIG. 3, or alternative variable domains that bind LGR5 set out herein. In certain aspects, the cancer expresses EGFR in sufficient levels for an antibody to bind the EGFR protein, such as an antibody comprising a VH chain of the variable domain that binds EGFR that comprises the amino acid sequence of the VH chain of MF3755 as depicted in FIG. 3, or alternative variable domains that bind EGFR set out herein.

### Mutations and Models

Cancers such as gastric, esophageal, gastro-esophageal-junction, head and neck cancer, non-small cell lung cancer or colorectal cancer can be related to the presence of mutations. Such mutations include mutations in known oncogenes such as PIK3CA, RAS, KRAS, HRAS, MAP2K1, RAF, BRAF and NOTCH1. Oncogenic mutations are generally described as activating mutations or mutations which result in new functions. Another type of cancer mutation involves tumor suppressor genes, such as TP53, MLH1, CDKN2A, and PTEN. Mutations in tumor suppressor genes are generally inactivating.

In certain aspects, the cancer and/or said subject having said cancer has mutations in one or more WNT, RTK/RAS, TP53 and/or TGF-ß signaling pathway genes.

In certain aspects, the cancer is colorectal cancer and is wildtype for RAS and/or RAF (including Kirsten Rat Sarcoma (KRAS) and/or B-Rapidly Accelerated Fibrosarcoma (BRAF)). In certain aspects, the genome of said colorectal cancer is wildtype for RAS and/or RAF, including KRAS and/or BRAF. In certain aspects, the cancer is mCRC and is wildtype for RAS and/or RAF (including Kirsten Rat Sarcoma (KRAS) and/or B-Rapidly Accelerated Fibrosarcoma (BRAF)). In certain aspects, the genome of said mCRC is wildtype for RAS and/or RAF, including KRAS and/or BRAF.

In certain aspects, for subjects suffering from mCRC, a screening step is included to detect (and optionally exclude subjects having) somatic mutations in RAS and RAF family genes (i.e., KRAS, NRAS, HRAS, BRAF, ARAF, RAF1). In certain aspects, a screening step is included to detect mutations or other oncogenic drivers associated with resistance to antibody-based EGFR inhibition, such as the EGFR ectodomain, or ERBB2/HER2 amplification.

In certain aspects, the cancer has an oncogenic mutation in one or more genes associated with colorectal cancer. In certain aspects, the cancer has an oncogenic mutation in a gene, and the corresponding encoded protein, selected from APC, FBXW7, KRAS, PIK3CA, SOX9, TP53, SMAD2, SMAD3, SMAD4, NRAS or TCF7L2. In certain aspects, the cancer has a mutation in a gene, and the corresponding encoded protein, selected from APC, FBXW7, KRAS, PIK3CA, SOX9, TP53, BRAF, SMAD2, SMAD3, SMAD4, NRAS or TCF7L2.

In certain aspects, the cancer has a mutation in a gene coding for APC. In certain aspects the mutation is a missense mutation, nonsense mutation, frameshift mutation, insertion, deletion or a mutation that leads to a copy number change. In certain aspects the mutation is a mono-allelic loss-of-function, a bi-allelic loss-of-function, or a loss-of-heterozygosity mutation. In certain aspects, the mutation is a bi-allelic loss-of-function. In certain aspects, APC comprises a mono-allelic loss-of-function missense mutation R653K in its protein structure, leading to a R>K amino acid change. In certain aspects the mutation is a loss-of-heterozygosity mutation leading to a copy number change. In certain aspects, APC comprises a bi-allelic loss-of-function mutation at position 4704, which causes a frameshift at position P1442fs*31 in its protein. In certain aspects, APC comprises a bi-allelic loss-of-function mutation at position 5040-5041, which leads to a frameshift at position T1556fs*3 in its protein. In certain aspects, APC comprises a mutation at position 4565-4566, which causes a frameshift at position F1396fs*1 in its protein.

Any mutation mentioned herein follows standard nomenclature as understood by the skilled person. As mentioned herein in the context of mutations, it will be clear to the skilled person that an asterisk (*) indicates the presence of a termination codon and thus termination of translation. Also, herein, frameshift mutations are described in the format X₁fs*X₂, wherein X₁ denotes the first affected amino acid of the protein and *X₂ denotes the position of the translation termination codon in the new reading frame.

In certain aspects, the cancer has a mutation in a gene coding for FBXW7. In certain aspects, the mutation is a missense mutation or a copy number change mutation. In certain aspects, the mutation is a mono-allelic loss-of-function or a loss-of-heterozygosity mutation.

In certain aspects, the cancer has a mutation in a gene coding for KRAS. In certain aspects the mutation is a missense mutation. In certain aspects the mutation is a gain-of-function mutation. In certain aspects KRAS comprises a mutation G12V in its protein structure, leading to a G>V amino acid change. In certain aspects KRAS comprises mutation Q61H in its protein structure, leading to a Q>H amino acid change. In certain aspects KRAS comprises a mutation G12S in its protein structure leading to a G>S amino acid change.

In certain aspects, the cancer has a mutation in a gene coding for PIK3CA. In certain aspects, the mutation is a missense mutation. In certain aspects, the mutation is a gain-of-function mutation. In certain aspects, PIK3CA comprises a gain-of-function missense mutation E545K in its protein structure, leading to a E>K amino acid change.

In certain aspects, the cancer has a mutation in a gene coding for SOX9. In certain aspects the mutation is a nonsense mutation or a frameshift mutation. In certain aspects the mutation is a mono-allelic loss-of-function. In certain aspects, SOX9 comprises a mono-allelic loss-of-function nonsense mutation Q412* in its protein structure, leading to a Q>* amino acid change. In certain aspects SOX9 comprises a mono-allelic loss-of-function mutation at position 1898-1899, which causes a frameshift at position *510fs*68 in its protein.

In certain aspects, the cancer has a mutation in a gene coding for TP53. In certain aspects the mutation is a missense mutation or a nonsense mutation. In certain aspects, the mutation is a mono-allelic loss-of-function or a bi-allelic loss-of-function. In certain aspects, TP53 comprises a missense mutation N239D in its protein structure, leading to a N>D amino acid change. In certain aspects, TP53 comprises a bi-allelic loss-of-function nonsense mutation Y126* in its protein structure, leading to a Y>* amino acid change. In certain aspects, TP53 comprises a mono-allelic loss-of-function missense mutation R282W in its protein structure, leading to a R>W amino acid change. In certain aspects, TP53 comprises a bi-allelic loss-of-function missense mutation M237I in its protein structure, leading to a M>I amino acid change.

In certain aspects, the cancer has a mutation in a gene coding for SMAD4. In certain aspects the mutation is a missense mutation or a loss-of-heterozygosity mutation. In certain aspects, SMAD4 comprises a missense mutation G510V in its protein structure, leading to a G>V amino acid change.

In certain aspects, the cancer has a mutation in a gene selected from SMAD2, SMAD3, SMAD4, NRAS or TCF7L2. In certain aspects the mutation is a loss-of heterozygosity mutation. In certain aspects the mutation leads to a copy number change.

In certain aspects, the present disclosure relates to a method of treatment of cancer in a subject having a mutation in one or more genes selected from APC, SOX9, KRAS, NRAS, SMAD2, or SMAD4. In certain aspects, the cancer has a mutation in the genes APC, SOX9, KRAS, NRAS, SMAD2, and SMAD4. In certain aspects, the mutation in the gene coding for APC is a mono-allelic loss-of-function missense mutation R653K in its protein structure, leading to a R>K amino acid change. In certain aspects the mutation in the gene coding for APC is a mutation at position 4565-4566, which causes a frameshift at position F1396fs*1 in its protein. In certain aspects, the mutation in the gene coding for SOX9 is a mono-allelic loss-of-function nonsense mutation Q412* in its protein structure, leading to a Q>* amino acid change. In certain aspects, the mutation in the gene coding for KRAS is a mutation G12V in its protein structure, leading to a G>V amino acid change. In certain aspects the mutation in genes NRAS, SMAD2, and SMAD4 is a loss-of heterozygosity mutation, leading to a copy number change.

In certain aspects, the present disclosure relates to a method of treatment of cancer in a subject having a mutation in APC.

In certain aspects, the present disclosure relates to a method of treatment of cancer in a subject having a mutation in one or more genes selected from TP53, APC, NRAS, SMAD2, SMAD4 or FBXW7. In certain aspects, the cancer has a mutation in the genes TP53, APC, NRAS, SMAD2, SMAD4 and FBXW7. In certain aspects, the mutation in the genes coding for APC, NRAS, SMAD2, SMAD4 and FBXW7 is a loss-of-heterozygosity mutation, leading to a copy number change. In certain aspects, the mutation in the gene coding for TP53 is a missense mutation N239D in its protein structure, leading to a N>D amino acid change. In certain aspects, the mutation in the gene coding for TP53 is a bi-allelic loss-of-function nonsense mutation Y126* in its protein structure, leading to a Y>* amino acid change.

In certain aspects, the present disclosure relates to a method of treatment of cancer in a subject having a mutation in one or more genes selected from TP53, APC, KRAS or PIK3CA. In certain aspects, the cancer has a mutation in the genes TP53, APC, KRAS and PIK3CA. In certain aspects, the mutation in the gene coding for APC is a bi-allelic loss-of-function mutation at position 4704, which causes a frameshift at position P1442fs*31 in its protein. In certain aspects, the mutation in the gene coding for KRAS is a mutation G12S in its protein structure leading to a G>S amino acid change. In certain aspects, the mutation in the gene coding for PIK3CA is a gain-of-function missense mutation E545K in its protein structure, leading to a E>K amino acid change. In certain aspects, the mutation in the gene coding for TP53 is a mono-allelic loss-of-function missense mutation R282W in its protein structure, leading to a R>W amino acid change.

In certain aspects, the present disclosure relates to a method of treatment of cancer in a subject having a mutation in one or more genes selected from TP53, APC, SOX9, KRAS, PIK3CA, TCF7L2, NRAS, SMAD2, SMAD3 or SMAD4. In certain aspects, the cancer has a mutation in the genes TP53, APC, SOX9, KRAS, PIK3CA, TCF7L2, NRAS, SMAD2, SMAD3 and SMAD4. In certain aspects, the mutation in the genes coding for APC, TCF7L2, NRAS, SMAD2, SMAD4 and TP53 is a loss-of-heterozygosity mutation, leading to a copy number change. In certain aspects, the mutation in the gene coding for APC is a bi-allelic loss-of-function mutation at position 5040-5041, which leads to a frameshift at position T1556fs*3 in its protein. In certain aspects, the mutation in the gene coding for SOX9 is a mono-allelic loss-of-function mutation at position 1898-1899, which causes a frameshift at position *510fs*68 in its protein. In certain aspects, the mutation in the gene coding for KRAS is a mutation Q61H in its protein structure, leading to a Q>H amino acid change. In certain aspects, the mutation in the gene coding for PIK3Ca is a gain-of-function missense mutation E545K in its protein structure, leading to a E>K amino acid change. In certain aspects, the mutation in the gene coding for SMAD4 is a missense mutation G510V in its protein structure, leading to a G>V amino acid change. In certain aspects, the mutation in the gene coding for TP53 is a bi-allelic loss-of-function missense mutation M237I in its protein structure, leading to a M>I amino acid change.

### Standard of Care Molecules of the Present Disclosure and Dosing Thereof

### Fluoropyrimidine

In certain aspects, the antibody or functional part, derivative and/or analogue thereof, is used in a treatment that further comprises administering a fluoropyrimidine. In certain aspects, the method of treating cancer in a subject further comprises administration of an effective amount of a fluoropyrimidine to the subject.

Fluoropyrimidines are anti-metabolites which inhibit thymidylate synthase, and lower the production of pyrimidine thymidine. Without being bound by theory, it is thought that fluoropyrimidines interfere with the synthesis of DNA and to a lesser extent, synthesis of RNA leading to cell death of rapidly growing cells.

Examples of suitable fluoropyrimidines in humans are capecitabine, carmofur (HCFU), doxifluridine, fluorouracil (5-FU) and tegafur. In certain aspects, a “fluoropyrimidine”, as used herein, includes, but is not limited to capecitabine, carmofur (HCFU), doxifluridine, fluorouracil (5-FU) and tegafur.

In certain aspects, the fluoropyrimidine is fluorouracil.

In certain aspects, the disclosure provides petosemtamab for use in the treatment of a cancer in a subject, wherein the treatment further comprises administering fluorouracil. In certain aspects the disclosure further provides a method of treating cancer in a subject, the method comprising administering an effective amount of petosemtamab and fluorouracil to the subject.

Fluorouracil is a fluoropyrimidine with the chemical formula 5-fluoro-2,4 (1H,3H)-pyrimidinedione. Fluorouracil is converted into three main active metabolites: 5-fluoro-2′-deoxyuridine-5′-monophosphate (FdUMP), 5-fluorouridine-5′-triphosphate (FUTP) and 5-fluoro-2′-deoxyuridine-5′-triphosphate (FdUTP). These metabolites have several effects including the inhibition of thymidylate synthase by FdUMP, incorporation of FUTP into RNA and incorporation of FdUTP into DNA.

Fluorouracil is known under various synonyms, the most common of which are 5-Fluoracil, 5-Fluoropyrimidine-2,4-dione, 5-FU and 5-Fluorouracil. Fluorouracil is sold under the brand name Adrucil among others. The IUPAC name of fluorouracil is 5-fluoropyrimidine-2,4(1H,3H)-dione. By intravenous injection, it is used for treatment of colorectal cancer, esophageal cancer, stomach cancer, pancreatic cancer, breast cancer and cervical cancer. As a cream it is used for actinic keratosis, basal cell carcinoma, and skin warts.

Fluorouracil and other human fluoropyrimidines have been used in the clinic for quite some time and appropriate regimens and dosage information is available to the person of ordinary skill in the art. Fluorouracil can be administered by intravenous injection as bolus, infusion or continuous infusion for up to several days. Fluorouracil can be administered as a monotherapy, in combination with leucovorin alone, or in combination with leucovorin and oxaliplatin or irinotecan. Fluorouracil can also be administered as a component of a cyclophosphamide-based multidrug regimen or as a component of a platinum-containing multidrug chemotherapy regimen.

In certain aspects, the dosing regimen of fluorouracil comprises administering fluorouracil in a range of 200-3000 mg/m² intravenously.

In certain aspects, the dosing regimen of fluorouracil comprises administering fluorouracil in combination with leucovorin alone, or in combination with leucovorin and oxaliplatin or irinotecan at 400 mg/m² by intravenous bolus on day1, followed by 2400 mg/m² to 3000 mg/m² intravenously as a continuous infusion over 46 hours every two weeks.

In certain aspects, the dosing regimen of fluorouracil comprises administering fluorouracil in combination with leucovorin, at 500 mg/m² by intravenous bolus on days 1, 8, 15, 22, 29 and 36 in 8-week cycles.

In certain aspects, the dosing regimen of fluorouracil comprises administering fluorouracil as a component of a cyclophosphamide-based multidrug regimen, at 500 mg/m² to 600 mg/m² intravenously on Days 1 and 8 every 28 days for 6 cycles.

In certain aspects, the dosing regimen of fluorouracil comprises administering fluorouracil as a component of a platinum-containing multidrug chemotherapy regimen, at 200 mg/m² to 1000 mg/m² intravenously as a continuous infusion over 24 hours.

In certain aspects, the dosing regimen of fluorouracil comprises administering fluorouracil in combination with leucovorin or as a component of a multidrug chemotherapy regimen that includes leucovorin, at 400 mg/m² by intravenous bolus on Day 1, followed by 2400 mg/m² intravenously as a continuous infusion over 46 hours every two weeks.

### TAS-102

In certain aspects, the antibody or functional part, derivative and/or analogue thereof is used in a treatment that further comprises administering a fluoropyrimidine. In certain aspects, the method of treating cancer in a subject, further comprises administration of an effective amount of a fluoropyrimidine.

In certain aspects, the antibody or functional part, derivative and/or analogue thereof is used in a treatment that further comprises administering a thymidine phosphorylase inhibitor. In certain aspects, the method of treating cancer in a subject further comprises administration of an effective amount of a thymidine phosphorylase.

In certain aspects, the antibody or functional part, derivative and/or analogue thereof is used in a treatment that further comprises administering a fluoropyrimidine and a thymidine phosphorylase inhibitor. In certain aspects, the method of treating cancer in a subject, further comprises administration of an effective amount of a fluoropyrimidine and an effective amount of a thymidine phosphorylase to the subject.

In certain aspects, the fluoropyrimidine is trifluridine. In certain aspects, the thymidine phosphorylase inhibitor is tipiracil hydrochloride.

In certain aspects, the disclosure provides petosemtamab for use in the treatment of a cancer in a subject, wherein the treatment further comprises administering trifluridine and tipiracil hydrochloride. In certain aspects the disclosure further provides a method of treating cancer in a subject, the method comprising administering an effective amount of petosemtamab, trifluridine and tipiracil hydrochloride to the subject.

In certain aspects, the combination of trifluorothymidine and tipiracil at a molar ratio of 1:0.5 is TAS-102.

Trifluridine, an antineoplastic thymidine-based nucleoside analogue, is described chemically as 2′-deoxy-5-(trifluoromethyl) uridine. Tipiracil hydrochloride, a thymidine phosphorylase inhibitor, is described chemically as 5-chloro-6-[(2-iminopyrrolidin-1-yl)methyl]pyrimidine-2,4-(1H,3H)-dione monohydrochloride or 2,4(1H,3H)-Pyrimidinedione, 5-chloro-6-[(2-imino-1-pyrrolidinyl)methyl]-, hydrochloride (1:1). TAS-102 comprises trifluridine, and tipiracil at a molar ratio of 1:0.5 (weight ratio, 1:00471). Without being bound by theory, it is thought that inclusion of tipiracil increases trifluridine exposure by inhibiting its metabolism by thymidine phosphorylase. Following uptake into cancer cells, trifluridine is incorporated into DNA, interferes with DNA synthesis and inhibits cell proliferation.

TAS-102 is known under various synonyms, the most common of which are Lonsurf, TAS 102, TAS-102 (Trifluridine/Tipiracil HCl), 4-hydroxy-1-((2R,4S,5R)-4-hydroxy-5-(hydroxymethyl)tetrahydrofuran-2-yl)-5-(trifluoromethyl)pyrimidin-2(1H)-one compound with 5-chloro-6-((2-iminopyrrolidin-1-yl)methyl)pyrimidine-2, 4-diol (1:1) hydrochloride, Viroptic mixture with 5-CIMU, Trifluridine-tipiracil hydrochloride mixture, Tipiracil/Trifluridine and EX-A1755. TAS-102 is sold under the brand name Lonsurf.

TAS-102 has been used in the clinic for quite some time and appropriate regimens and dosage information is available to the person of ordinary skill in the art. TAS-102 is indicated as monotherapy for the treatment of adult patients with metastatic colorectal cancer (CRC) who have been previously treated with, or are not considered candidates for, available therapies including fluoropyrimidine-, oxaliplatin- and irinotecan-based chemotherapies, anti-VEGF agents, and anti-EGFR agents. TAS-102 is also indicated as monotherapy for the treatment of adult patients with metastatic gastric cancer including adenocarcinoma of the gastroesophageal junction, who have been previously treated with at least two prior systemic treatment regimens for advanced disease.

In certain aspects, the dosing regimen of TAS-102 comprises administering trifluorothymidine and tipiracil at a molar ratio of 1:0.5 at 35-80 mg/m² twice daily.

In certain aspects, the dosing regimen of TAS-102 comprises administering trifluorothymidine and tipiracil at a molar ratio of 1:0.5, at a starting dose of 35 mg/m²/dose up to a maximum of 80 mg/dose orally twice daily on Days 1 to 5 and Days 8 to 12 of each 28-day cycle as long as benefit is observed or until unacceptable toxicity occurs.

### Oxaliplatin

In certain aspects, the antibody or functional part, derivative and/or analogue thereof is used in a treatment that further comprises administering of a platinum-based chemotherapeutic agent. In certain aspects, the method of treating cancer in a subject, further comprises administration of an effective amount of a platinum-based chemotherapeutic agent to the subject.

Platinum-based chemotherapeutic agents are antineoplastic drugs widely used to treat cancer. They are coordination complexes of platinum. Without being bound to theory, it is thought that platinum-based chemotherapeutic agents cause crosslinking of DNA which inhibits DNA repair and/or DNA synthesis.

Examples of suitable platinum-based chemotherapeutic agents in humans are cisplatin, oxaliplatin, carboplatin, nedaplatin, triplatin tetranitrate, phenanthriplatin, picoplatin and satraplatin. In certain aspects, a “platinum-based chemotherapeutic agent”, as used herein, includes, but is not limited to cisplatin, oxaliplatin, carboplatin, nedaplatin, triplatin tetranitrate, phenanthriplatin, picoplatin and satraplatin. While cisplatin, oxaliplatin, nedaplatin and carboplatin are approved and widely used for the treatment of several different cancer indications, triplatin tetranitrate, phenanthriplatin, picoplatin and satraplatin are currently in clinical trials.

In certain aspects, the platinum-based chemotherapeutic agent is cisplatin, oxaliplatin or carboplatin. In certain aspects, the platinum-based chemotherapeutic agent is oxaliplatin.

In certain aspects, the disclosure provides petosemtamab for use in the treatment of a cancer in a subject, wherein the treatment further comprises administering oxaliplatin. In certain aspects the disclosure further provides a method of treating cancer in a subject, the method comprising administering an effective amount of petosemtamab and oxaliplatin to the subject.

Oxaliplatin is a platinum-based drug with the molecular formula C₈H₁₄N₂O₄Pt and the chemical name of cis-[(1 R,2 R)-1,2-cyclohexanediamine-N,N′][oxalato(2-)-0,0]platinum. Oxaliplatin is an organoplatinum complex in which the platinum atom is complexed with 1,2-diaminocyclohexane (DACH) and with an oxalate ligand as a leaving group. Oxaliplatin undergoes nonenzymatic conversion in physiologic solutions to active derivatives via displacement of the labile oxalate ligand. These derivatives form inter- and intra-strand DNA crosslinks that inhibit DNA replication and transcription.

Oxaliplatin is known under various synonyms, the most common of which are Diaminocyclohexane Oxalatoplatinum, L-OHP, Oxalatoplatin, Oxalatoplatinum, Oxaliplatin, Dacplat, Eloxatin, or Elplat. The trade name of oxaliplatin is Eloxatin. Oxaliplatin, in combination with fluorouracil and folinic acid is indicated for adjuvant treatment of state III (Duke's C) colon cancer, after complete resection of primary tumor and metastatic colorectal cancer.

Oxaliplatin and other platinum-base chemotherapeutic agents have been used in the clinic for quite some time and appropriate regimens and dosage information is available to the person of ordinary skill in the art. In certain aspects, the dosing regimen of oxaliplatin comprises administering oxaliplatin in a range of about 65-160 mg/m².

In certain aspects, the dosing regimen of oxaliplatin comprises administering oxaliplatin at 85 mg/m² every two weeks.

In certain aspects, oxaliplatin is administered in combination with fluorouracil and leucovorin every two weeks.

In certain aspects, oxaliplatin is administered at 85 mg/m² in combination with leucovorin at 200 mg/m² as an intravenous infusion over 120 minutes followed by fluorouracil at 400 mg/m² as intravenous bolus over 2-4 minutes, followed by fluorouracil at 600 mg/m² as a 22-hour continuous infusion.

### Venetoclax

In certain aspects, the antibody or functional part, derivative and/or analogue thereof is used in a treatment that further comprises administering a BCL-2 inhibitor. In certain aspects, the method of treating cancer in a subject, further comprises administration of an effective amount of a BCL-2 inhibitor to the subject.

B-cell lymphoma 2 (BCL-2) is a part of a family of regulators of apoptosis. BCL-2 is a pro-survival protein which protect cells from apoptosis when overexpressed. BCL-2 protein is overexpressed in many cancers and plays an important role in the negative regulation of apoptosis. Its expression is associated with increased drug resistance and tumor cell survival.

BH3-mimetics comprise a class of BCL-2 inhibitors that have shown promising results in several hematological malignancies, both as single agents and in combination with other anti-cancer drugs. Without being bound by theory, it is thought that BH3-mimetics inhibit the activity of BCL-2 and restore apoptotic processes in tumor cells. Examples of suitable BCL-2 inhibitors in humans are ABT-737, navitoclax (ABT-263), and venetoclax.

In certain aspects, a “BCL-2 inhibitor”, as used herein, includes, but is not limited to ABT-737, navitoclax (ABT-263), or venetoclax In certain aspects, a BLC-2 inhibitor is venetoclax.

In certain aspects, the disclosure provides petosemtamab for use in the treatment of a cancer in a subject, wherein the treatment further comprises administering venetoclax. In certain aspects the disclosure further provides a method of treating cancer in a subject, the method comprising administering an effective amount of petosemtamab and venetoclax to the subject.

Venetoclax is a potent, selective inhibitor of BCL-2, an anti-apoptotic protein. Venetoclax binds directly to the BH3-binding groove of BCL-2, displacing BH3 motif-containing pro-apoptotic proteins like BIM, to initiate mitochondrial outer membrane permeabilization (MOMP), caspase activation, and programmed cell death. In non-clinical studies, venetoclax has demonstrated cytotoxic activity in tumor cells that overexpress BCL-2.

Venetoclax is known under various synonyms, the most common of which are Venclexta, Venclyxto, ABT-199, GDC-0199, ABT199, ABT 199, and RG7601. Venclexta is indicated for the treatment of patients with chronic lymphocytic leukemia (CLL) or small lymphocytic lymphoma (SLL). Venclexta is also indicated in combination with azacitidine or decitabine or low-dose cytarabine for the treatment of newly-diagnosed acute myeloid leukemia (AML) in adults who are ineligible for intensive chemotherapy.

Venetoclax has been used in the clinic for quite some time and appropriate regimens and dosage information is available to the person of ordinary skill in the art.

In certain aspects, venetoclax is administered daily in at 20-1200 mg daily.

In certain aspects, venetoclax is administered in a ramp-up or step-up dosing regimen. In certain aspects, the initial dose of venetoclax is gradually increased in ramp-up doses, until the full dose is reached. In certain aspects, the ramp up doses are administered at regular intervals.

In certain aspects, the initial dose of venetoclax is 20 mg administered in week1, followed by 50 mg in week2, followed by 100 mg in week3, followed by 200 mg in week4, followed by 400 mg in week5.

In certain aspects, venetoclax is administered daily at 400 mg, after following the ramp-up dosing schedule as described herein. In certain aspects, venetoclax is administered at 400 mg once daily until disease progression or unacceptable toxicity is observed.

SN-38 In certain aspects, the antibody or functional part, derivative and/or analogue thereof is used in a treatment that further comprises administering of SN-38. In certain aspects, the method of treating cancer in a subject, further comprises administration of an effective amount of SN-38 to the subject.

SN-38 is 7-ethyl-10-hydroxy-camptothecin, a compound that inhibits the activity of DNA topoisomerase 1. It is the biologically active and water-insoluble metabolite of irinotecan (CPT-11). Without being bound by theory, it is thought that irinotecan is converted to SN-38 by carboxylesterases in the liver and the tumor. SN-38 has been demonstrated to exhibit up to a 1,000-fold more potent cytotoxic activity than irinotecan against various cancer cells in vitro. The metabolic conversion rate is, however, very low, with only <10% of the original volume of irinotecan being metabolized to SN-38; conversion of irinotecan to SN-38 also depends on genetic inter-individual variability of the activity of carboxylesterases. Direct administration of SN-38 itself for clinical cancer treatment eliminates the need for conversion. SN-38 is also known to be identical in terms of both efficacy and toxicity to irinotecan (Nakajima T E, et al. Int J Cancer. 2008 May 1; 122(9):2148-53). A skilled person will appreciate that reference herein to “administration of SN-38 to a subject” does not encompass administration of irinotecan to a subject which may then be converted to SN-38. A skilled person will further appreciate that all references herein disclosing the “administration of SN-38 to a subject” also include the administration of a pharmaceutical composition comprising SN-38 to a subject.

SN-38 is known under various synonyms, the most common of which are 7-Ethyl-10-hydroxycamptothecin, SN-38, SN38, SN-38 lactone, NK012, NK-012, 7-Ethyl-10-hydroxy-20(S)-camptothecin and 10-Hydroxy-7-ethylcamptothecin. Its IUPAC name is (4S)-4,11-diethyl-4,9-dihydroxy-1,4-dihydro-3H,14H-pyranof[3′, 4′:6,7]indolizino[1,2-b]quinoline-3,14-dione. LE-SN-38 is a novel liposome based formulation containing liposomes of uniform size distribution (<200 nm). Drug entrapment efficiency of the formulation is >95%.

In certain aspects, the disclosure provides petosemtamab for use in the treatment of a cancer in a subject, wherein the treatment further comprises administering SN-38. In certain aspects the disclosure further provides a method of treating cancer in a subject, the method comprising administering an effective amount of petosemtamab and SN-38 to the subject.

In certain aspects, SN-38 is administered as encapsulated or conjugated into a suitable carrier, including a soluble polymer, a liposome, a micelle, an antibody, a peptide, a polymer-drug conjugate, a nanoparticle or a polymeric implant.

### FOLFOX

In certain aspects, the antibody or functional part, derivative and/or analogue thereof is used in a treatment that further comprises administering of oxaliplatin, folinic acid (leucovorin) and fluorouracil. In certain aspects, the method of treating cancer in a subject, further comprises administration of an effective amount of oxaliplatin, folinic acid and fluorouracil to the subject.

In certain aspects, the disclosure provides petosemtamab for use in the treatment of a cancer in a subject, wherein the treatment further comprises administering FOLFOX. In certain aspects the disclosure further provides a method of treating cancer in a subject, the method comprising administering an effective amount of petosemtamab and FOLFOX to the subject.

In certain aspects, the combination of oxaliplatin, folinic acid and fluorouracil is the FOLFOX regimen. FOLFOX is known under various synonyms, the most common of which are FOLFOX protocol, FOLFOX regimen, FOLFOX-4, FOLFOX-6, FOLFOX-7 and Fluorouracil regimen with leucovorin calcium and oxaliplatin. Folinic acid is also known under the synonyms leucovorin, FA or calcium folinate.

In certain aspects, the disclosure provides petosemtamab for use in the treatment of a cancer in a subject, wherein the treatment further comprises administering oxaliplatin, folinic acid and fluorouracil. In certain aspects the disclosure further provides a method of treating cancer in a subject, the method comprising administering an effective amount of petosemtamab, oxaliplatin, folinic acid and fluorouracil to the subject.

In certain aspects, the antibody or functional part, derivative and/or analogue thereof, is used in a treatment that further comprises administering FOLFOX (fluorouracil, leucovorin, and oxaliplatin). In certain aspects, said subject or cancer as treated is wildtype for RAS and/or RAF (including Kirsten Rat Sarcoma (KRAS) and/or B-Rapidly Accelerated Fibrosarcoma (BRAF)). In certain aspects, the genome of said mCRC is wildtype for RAS and/or RAF, including KRAS and/or BRAF. In certain aspects, subjects to be administered FOLFOX, have had only a single prior anti-cancer treatment, such as a chemotherapy for the metastatic setting, such as first line fluoropyrimidine-irinotecan-based chemotherapy with or without bevacizumab.

In certain aspects, the dosing regimen of FOLFOX comprises administering oxaliplatin together with folinic acid intravenously, followed by fluorouracil intravenously.

In certain aspects, the dosing regimen of FOLFOX comprises administering oxaliplatin at 50-200 mg/m² together with folinic acid at 200-600 mg/m² intravenously, followed by fluorouracil at 1200-3600 mg/m² intravenously.

In certain aspects, the dosing regimen of FOLFOX comprises administering oxaliplatin at 85 mg/m² together with folinic acid at 400 mg/m² intravenously, followed by fluorouracil at 2400 mg/m².

In certain aspects, the dosing regimen of FOLFOX comprises: Day 1 oxaliplatin at 85 mg/m² together with folinic acid at 200 mg/m² both administered in a 2 hour infusion, followed by fluorouracil at 400 mg/m² bolus, followed by fluorouracil at 600 mg/m² in a 22 hour infusion; Day 2 folinic acid at 200 mg/m² administered in a 2 hour infusion, followed by fluorouracil at 400 mg/m² bolus, followed by fluorouracil 600 mg/m² in a 22 hour infusion. In certain aspects, dosing of FOLFOX is repeated every 2 weeks and followed for 12 cycles.

In certain aspects, the dosing regimen of FOLFOX comprises administering, on day 1 of a two-week cycle: Oxaliplatin at 85 mg/m² (e.g. as an IV infusion given over 2 h), followed by or concurrent with leucovorin at 200 mg/m² (e.g. as IV infusion over 2 h), followed by 5-FU as a 400 mg/m² (e.g. bolus IV), followed by 5-FU at 600 mg/m² (e.g. given as a continuous infusion over 22 h). In certain aspects, the dosing regimen of FOLFOX further comprises administering, on day 2 of a two-week cycle: leucovorin 200 mg/m² (e.g. as an IV infusion over 2 h), followed by 5-FU at 400 mg/m² (e.g., as bolus IV), and followed by 5-FU 600 mg/m² (e.g., given as a continuous infusion over 22 h).

In certain aspects, the dosing regimen of FOLFOX comprises oxaliplatin at 85 mg/m², bolus of fluorouracil at 400 mg/m² and folinic acid at 400 mg/m², administered in a 2 hour infusion on day 1, followed by fluorouracil at 2400 mg/m² in a 46 hour continuous infusion.

In the event that the subject experiences an oxaliplatin-related adverse reactions (also known as adverse events, or AEs) event following commencement of any of the relevant methods described above, the methods may involve adaptation of dose regimens based on approved prescribing information for oxaliplatin. Dosage modifications for adverse reactions for advanced colorectal cancer are presented in Table 1.

TABLE 1
Recommended Dose Modifications for Oxaliplatin Drug-related adverse reactions,
FDA approved prescribing information for Eloxatin (oxaliplatin).

| Adverse | | Oxaliplatin Dosage |
|---|---|---|
| Reactions | Severity | Modifications |
| Neuropathy | Persistent Grade 2 | Consider reducing oxaliplatin dose |
| | | to 65 mg/m² |
| | Persistent Grade 3 | Consider discontinuing oxaliplatin |
| | Grade 4 | Discontinue oxaliplatin |
| Myelosuppression | Grade 4 neutropenia or | Delay the next dose until |
| | febrile neutropenia | neutrophils ≥1.5 × 10⁹/L and |
| | Grades 3-4 | platelets ≥75 × 10⁹/L. |
| | thrombocytopenia | Reduce oxaliplatin dose to 65 mg/m² |
| Gastrointestinal | Grades 3-4 | After recovery, reduce oxaliplatin |
| Adverse Reactions | | dose to 65 mg/m² along with a dose |
| | | reduction of fluorouracil to |
| | | 300 mg/m² as an intravenous bolus |
| | | and 500 mg/m² as a 22-hour |
| | | continuous infusion |

Management of the adverse events may be tailored based on the severity of the adverse events. Specific measures of dose adaptation and support, as specified in the prescribing information for oxaliplatin, are provided below for the following events: hypersensitivity reactions, peripheral sensory neuropathy, acute neuropathy, delayed neuropathy, severe myelosuppression, posterior reversible encephalopathy syndrome (PRES), pulmonary toxicity, hepatotoxicity, QT interval prolongation and ventricular arrhythmias, rhabdomyolysis, and hemorrhage.

In certain aspects, the recommended dose modifications for oxaliplatin drug-related adverse events follow Table 1 the FDA-approved prescribing information for Eloxatin (oxaliplatin injection for intravenous use, revision 6/2023 or as current at the time of administration).

In certain aspects, dose modifications at the start of subsequent cycles of therapy are modified relative to the starting dose used in the preceding cycle following Tables 2 and 3 if a subject experiences toxicities as mentioned in said Tables.

In certain aspects, if a subject experiences acute and delayed neuropathy, the dose of oxaliplatin is reduced or permanently discontinued, for persistent neurosensory reactions based on the severity of the adverse reaction, following the schedule of Table 1.

In certain aspects, if a subject experiences a persistent grade 2 neuropathy, following administration of oxaliplatin as part of the FOLFOX regimen, oxaliplatin dosing is 65 mg/m². In certain aspects, if a subject experiences persistent grade 3 neuropathy, oxaliplatin is discontinued. In certain aspects, if a subject experiences grade 4 neuropathy, oxaliplatin is discontinued.

In certain aspects, if a subject experiences grade 4 neutropenia or febrile neutropenia, dosing is delayed until neutrophils are >1.5×10⁹/L and platelets are ≥75×10⁹/L. In certain aspects, oxaliplatin dose is (reduced to) 65 mg/m². In certain aspects, if the subject has grade 4 thrombocytopenia, dosing is delayed until neutrophils are ≥1.5×10⁹/L and platelets are ≥75×10⁹/L. In certain aspects, oxaliplatin dose is (reduced to) 65 mg/m².

In certain aspects, if a subject experiences grade 3-4 gastrointestinal adverse reaction, following administration of oxaliplatin as part of the FOLFOX regimen, oxaliplatin dosage is reduced, after recovery to 65 mg/m² along with a dose reduction of fluorouracil to 300 mg/m² as an intravenous bolus and 500 mg/m² as a 22-hour continuous infusion.

### FOLFIRI

In certain aspects, the antibody or functional part, derivative and/or analogue thereof is used in a treatment that further comprises administering of irinotecan, folinic acid and fluorouracil. In certain aspects, the method of treating cancer in a subject, further comprises administration of an effective amount of irinotecan, folinic acid (leucovorin) and fluorouracil to the subject.

In certain aspects, the disclosure provides petosemtamab for use in the treatment of a cancer in a subject, wherein the treatment further comprises administering FOLFIRI. In certain aspects the disclosure further provides a method of treating cancer in a subject, the method comprising administering an effective amount of petosemtamab and FOLFIRI to the subject.

In certain aspects, the combination of irinotecan, folinic acid and fluorouracil is the FOLFIRI regimen. FOLFIRI is known under various synonyms, the most common of which are FOLFIRI protocol, FOLFIRI regimen, 5-Fluorouracil/Folinic acid/irinotecan and Irinotecan, Leucovorin and Fluorouracil mixture.

In certain aspects, the antibody or functional part, derivative and/or analogue thereof, is used in a treatment that further comprises administering fluorouracil, leucovorin, and irinotecan (FOLFIRI). In certain aspects, said subject or cancer as treated is wildtype for RAS and/or RAF (including Kirsten Rat Sarcoma (KRAS) and/or B-Rapidly Accelerated Fibrosarcoma (BRAF)). In certain aspects, the genome of said mCRC is wildtype for RAS and/or RAF, including KRAS and/or BRAF. In certain aspects, subjects administered with FOLFIRI have had only a single prior anti-cancer treatment, such as a chemotherapy for the metastatic setting. In certain aspects, said prior treatment comprises fluoropyrimidine-oxaliplatin-based chemotherapy with or without bevacizumab.

In certain aspects, the disclosure provides petosemtamab for use in the treatment of a cancer in a subject, wherein the treatment further comprises administering irinotecan, folinic acid and fluorouracil. In certain aspects the disclosure further provides a method of treating cancer in a subject, the method comprising administering an effective amount of petosemtamab, irinotecan, folinic acid and fluorouracil to the subject.

In certain aspects, the dosing regimen of FOLFIRI comprises administering irinotecan together with folinic acid intravenously, followed by fluorouracil.

In certain aspects, the dosing regimen of FOLFIRI comprises administering irinotecan at 180 mg/m² together with folinic acid at 200-400 mg/m² intravenously, followed by fluorouracil at 400-2400 mg/m² intravenously.

In certain aspects, the dosing regimen of FOLFIRI comprises administering irinotecan at 180 mg/m² together with folinic acid at 200 mg/m² or 400 mg/m² intravenously, followed by fluorouracil at 400 mg/m² bolus, followed by fluorouracil at 2400 mg/m² as a 46 hour continuous infusion. In certain aspects, dosing of FOLFIRI is repeated every 14 days.

In certain aspects, FOLFIRI regimen comprises administering on Day 1 of 2-week cycle—irinotecan at 180 mg/m² given as IV infusion over 90 minutes, followed by or concurrent with folinic acid (leucovorin) at 200 mg/m² IV infusion over 2 hr, followed by fluorouracil at 400 mg/m² IV bolus, followed by fluorouracil at 600 mg/m² given as a 22 hour continuous infusion. Day2 of 2-week cycle—folinic acid (leucovorin) at 200 mg/m² IV infusion over 2 hr, followed by fluorouracil at 400 mg/m² IV bolus, followed by fluorouracil at 600 mg/m² given as a 22 hour continuous infusion.

In the event that the subject has an drug-related adverse event (AEs) following commencement ofany of the methods described above, the methods may involve adaptation of dose regimens based on approved prescribing information for irinotecan (also known as irinotecan hydrochloride or Camptosar). Dosage modifications for adverse reactions for advanced colorectal cancer are presented in Table 2.

TABLE 2
Recommended Dose Modifications for irinotecan Drug-related AEs.

| Toxicity | | At the Start of |
|---|---|---|
| NCI CTC Grade1¹ | | Subsequent Cycles |
| (Value) | During a Cycle of Therapy | of Therapy² |
| No toxicity | Maintain dose level | Maintain dose level |
| Neutropenia | | |
| 1 (1500 to 1999/mm³) | Maintain dose level | Maintain dose level |
| 2 (1000 to 1499/mm³) | decrease 1 dose level | Maintain dose level |
| 3 (500 to 999/mm³) | Omit dose until resolved to ≤ Grade 2, | decrease 1 dose level |
| | then decrease 1 dose level | |
| 4 (<500/mm³) | Omit dose until resolved to ≤ Grade 2, | decrease 2 dose levels |
| | then decrease 2 dose levels | |
| Neutropenic fever | Omit dose until resolved, then decrease 2 dose levels | |
| Other hematologic | Dose modifications for leukopenia or thrombocytopenia during a cycle | |
| toxicities | of therapy and at the start of subsequent cycles of therapy are also | |
| | based on NCI toxicity criteria and are the same as recommended for | |
| | neutropenia above. | |
| Diarrhea | | |
| 1 (2-3 stools/day > | Delay dose until resolved to baseline, | Maintain dose level |
| pretx³) | then give same dose | |
| 2 (4-6 stools/day > pretx) | Omit dose until resolved to baseline, then | Maintain dose level |
| | decrease 1 dose level | |
| 3 (7-9 stools/day > pretx) | Omit dose until resolved to baseline, then | decrease 1 dose level |
| | decrease 1 dose level | |
| 4 (≥10 stools/day > pretx) | Omit dose until resolved to baseline, then | decrease 2 dose levels |
| | decrease 2 dose levels | |
| Other nonhematologic | | |
| toxicities⁴ | | |
| 1 | Maintain dose level | Maintain dose level |
| 2 | Omit dose until resolved to ≤Grade 1, | Maintain dose level |
| | then decrease 1 dose level | |
| 3 | Omit dose until resolved to ≤Grade 2, | decrease 1 dose level |
| | then decrease 1 dose level | |
| 4 | Omit dose until resolved to ≤Grade 2, | decrease 2 dose levels |
| | then decrease 2 dose levels | |
| | For mucositis/stomatitis decrease only 5- | For mucositis/stomatitis |
| | FU, not irinotecan. | decrease only 5-FU, not |
| | | irinotecan. |

Source: Approved prescribing information for irinotecan.
¹National Cancer Institute-Common Toxicity Criteria (version 1.0)
²Relative to the starting dose used in the previous cycle
³Pre-treatment
⁴Excludes alopecia, anorexia, asthenia

TABLE 3
Starting Dose and Modified Dose Levels for FOLFIRI.

| Chemotherapy | Starting | Dose Level | Dose Level |
|---|---|---|---|
| Agent | Dose | Reduction 1 | Reduction 2 |
| Camptosar/irinotecan | 180 | 150 | 120 |
| hydrochloride | | | |
| LV | 200 | 200 | 200 |
| 5-FU Bolus | 400 | 320 | 240 |
| 5-FU Infusion | 600 | 480 | 360 |

Source: FDA approved prescribing information for Camptosar (irinotecan hydrochloride).
Camptosar = irinotecan hydrochloride; 5-FU = 5-Fluorouracil; FOLFIRI = 5-FU, LV, and irinotecan; LV = Leucovorin.

In certain aspects, grading of adverse events (or also used herein as toxicities or PG-3T adverse reactions) and their definitions follow Table 2, the National Cancer Institute-Common Terminology Criteria for Adverse Events (NCI-CTCAE) v.4.03/v5.0, NCI-CTC v1.0 or as current at the time of administration.

In certain aspects, the recommended dose modifications for irinotecan drug-related adverse events (AEs) are as described in National Cancer Institute (NCI)-Common Toxicity Criteria (version 1.0).

In certain aspects, the recommended dose modifications for irinotecan drug-related adverse events follow approved prescribing information for Camptosar (revision 1/2022 injection for intravenous use or as current at the time of administration).

In certain aspects, dose modifications at the start of subsequent cycles of therapy are modified relative to the starting dose used in the preceding cycle following Table 2 and 3 if a subject experiences toxicities as mentioned in said tables.

In certain aspects, if a subject does not experience any toxicity, the dose level as administered is maintained during a cycle of therapy. In certain aspects, if a subject does not experience any toxicity, the dose level is maintained at the starting dose for subsequent cycles of therapy.

In certain aspects, if a subject experiences grade 1 neutropenia (1500 to 1999/mm³), the dose level as administered is maintained during a cycle of therapy. In certain aspects, said dose level is maintained at the start of subsequent cycles of therapy.

In certain aspects, if a subject experiences grade 2 neutropenia (1000 to 1499/mm³), the dose level as administered is decreased by 1 dose level during a cycle of therapy. In certain aspects, said dose level is maintained at the start of subsequent cycles of therapy.

In certain aspects, if a subject experiences grade 3 neutropenia (500 to 999/mm³), the dose level as administered is omitted until resolved to ≤grade 2, then decreased by 1 dose level, during a cycle of therapy. In certain aspects, said dose level is decreased by 1 dose level at the start of subsequent cycles of therapy.

In certain aspects, if a subject experiences grade 4 neutropenia (<500 mm³), the dose as administered is omitted until resolved to ≤grade 2, then decreased by 2 dose levels, during a cycle of therapy. In certain aspects, said dose level is decreased by 2 dose level at the start of subsequent cycles of therapy.

In certain aspects, if the subject has neutropenic fever, the dose is omitted until resolved to ≤grade 2, then decreased by 2 dose levels.

In certain aspects, if a subject experiences other hematologic toxicities, dose modifications during a cycle of therapy and at the start of subsequent cycles of therapy are also based on NCI toxicity criteria and are the same as recommended for neutropenia above. In certain aspects, if the subject has leukopenia or thrombocytopenia, dose modifications during a cycle of therapy and at the start of subsequent cycles of therapy are also based on NCI toxicity criteria and are the same as recommended for neutropenia above.

In certain aspects, if a subject experiences grade 1 diarrhea (2-3 stools/day>pretreatment), the dose as administered is delayed until resolved to baseline, then the subject is given the same dose, during a cycle of therapy. In certain aspects, said dose as administered is maintained at the start of subsequent cycles of therapy.

In certain aspects, if a subject experiences grade 2 diarrhea (4-6 stools/day>pretreatment), the dose as administered is omitted until resolved to baseline, then decreased by 1 dose level, during a cycle of therapy. In certain aspects, said dose as administered is maintained at the start of subsequent cycles of therapy.

In certain aspects, if a subject experiences grade 3 diarrhea (7-9 stools/day>pretreatment), the dose as administered is omitted until resolved to baseline, then decreased by 1 dose level, during a cycle of therapy. In certain aspects, said dose as administered is decreased by 1 dose level at the start of subsequent cycles of therapy.

In certain aspects, if a subject experiences grade 4 diarrhea (>10 stools/day>pretreatment), the dose as administered is omitted until resolved to baseline, then decreased by 2 dose levels, during a cycle of therapy. In certain aspects, said dose as administered is decreased by 2 dose levels at the start of subsequent cycles of therapy.

In certain aspects, a subject may experience other nonhematologic toxicities. In certain aspects, other nonhematologic toxicities exclude alopecia, anorexia and asthenia.

In certain aspects, if a subject experiences other nonhematologic toxicities (grade 1), the dose as administered is maintained during a cycle of therapy. In certain aspects, said dose as administered is maintained at the start of subsequent cycles of therapy.

In certain aspects, if a subject experiences other nonhematologic toxicities (grade 2), the dose as administered is omitted until resolved to grade 1, then decreased by 1 dose level, during a cycle of therapy. In certain aspects, said dose as administered is maintained at the start of subsequent cycles of therapy.

In certain aspects, if a subject experiences other nonhematologic toxicities (grade 3), the dose as administered is omitted until resolved to grade 2, then decreased by 1 dose level, during a cycle of therapy. In certain aspects, said dose as administered is decreased by 1 dose level at the start of subsequent cycles of therapy.

In certain aspects, if a subject experiences other nonhematologic toxicities (grade 4), the dose as administered is omitted until resolved to grade 2, then decreased by 2 dose levels, during a cycle of therapy. In certain aspects, said dose as administered is decreased by 2 dose levels at the start of subsequent cycles of therapy.

In certain aspects, if a subject experiences mucositis or stomatitis, the dose as administered is decreased only for 5-FU and not for irinotecan, during a cycle of therapy. In certain aspects, the dose as administered is decreased only for 5-FU and not for irinotecan, at the start of subsequent cycles of therapy.

### Multispecific/Bispecific Antibodies

In certain aspects, the antibody or functional part, derivative and/or analogue thereof as disclosed herein is a multispecific antibody. In certain aspects, said antibody is a bispecific antibody. Said multi- or bispecific antibody or a functional part, derivative and/or analogue thereof, in certain aspects comprises a first variable domain that binds an extracellular part of the epidermal growth factor (EGF) receptor and a second variable domain, which in certain aspects, does not bind EGFR. In certain aspects, the antibody or functional part, derivative and/or analogue thereof binds EGFR monovalently. Also in certain aspects, said multispecific or bispecific antibody or functional part, derivative and/or analogue thereof, comprises a second variable domain that binds LGR5. In certain aspects, said antibody or functional part, derivative and/or analogue thereof that comprises a variable domain that binds an extracellular part of EGFR, and optionally comprises a variable domain that binds an extracellular part of LGR5, is or comprises petosemtamab.

### EGFR

Epidermal growth factor (EGF) receptor (EGFR, ErbB1, or HER1) is a member of a family of four receptor tyrosine kinases (RTKs), named Her- or cErbB-1, -2, -3 and -4. EGFR is known under various synonyms, the most common of which is EGFR. EGFR has an extracellular domain (ECD) that is composed of four sub-domains, two of which are involved in ligand binding and two of which are involved in homo-dimerisation and hetero-dimerisation. EGFR integrates extracellular signals from a variety of ligands to yield diverse intracellular responses. A major signal transduction pathway activated by EGFR is composed of the Ras-mitogen-activated protein kinase (MAPK) mitogenic signaling cascade. Activation of this pathway is initiated by the recruitment of Grb2 to tyrosine phosphorylated EGFR. This leads to activation of Ras through the Grb2-bound Ras-guanine nucleotide exchange factor Son of Sevenless (SOS). In addition, the PI3-kinase-Akt signal transduction pathway is also activated by EGFR, although this activation is much stronger in case there is co-expression of ErbB-3 (HER3). EGFR is implicated in several human epithelial malignancies, notably cancers of the breast, bladder, non-small cell lung cancer lung, colon, ovarian head and neck and brain. Activating mutations in the gene have been found, as well as over-expression of the receptor and of its ligands, giving rise to autocrine activation loops. This RTK has therefore been extensively used as target for cancer therapy.

Both small-molecule inhibitors targeting the RTK and monoclonal antibodies (mAbs) directed to the extracellular ligand-binding domains have been developed and have shown hitherto several clinical successes, albeit mostly for a select group of patients. The database accession number for the human EGFR protein and the gene encoding it is GenBank NM_005228.3. This accession number is primarily given to provide a further method of identification of EGFR protein as a target, the actual sequence of the EGFR protein bound by an antibody may vary, for instance because of a mutation in the encoding gene such as those occurring in some cancers or the like.

Where reference herein is made to EGFR, the reference refers to human EGFR unless otherwise stated. The variable domain antigen-binding site that binds EGFR, binds EGFR and a variety of variants thereof such as those expressed on some EGFR positive tumors.

In certain aspects, the EGFR is a human EGFR. The EGFR that is bound by said antibody or functional part, derivative and/or analogue thereof of the present disclosure, includes wildtype EGFR as well as EGFR having an oncogenic driver mutation. In certain aspects, said oncogenic driver mutation is an activating EGFR mutation. In certain aspects, such a mutation does not conformationally change the epitope that is bound by the antibody of the present disclosure. In certain aspects, the EGFR mutations of the present disclosure include mutations such as exon 18 mutations, including G719A, G719C, 2E709_T710D, E709A, G719S; exon 19 deletion mutations, including deletion of LREA or VAIKEL; exon 19 point mutations G735S, P753L, L747S, D761Y; in-frame exon 20 insertion mutations of 1-7 amino acids, exon 20 point mutations, including V765A, T783A, V774A, S784P, V769M, T790M; exon 21 mutations, including L858R, T854A, A871E, L861A, L861C, L861S, V843I or P848L. The antibody of the present disclosure binds an epitope that is not located in close proximity of said mutations. In particular, the EGFR mutation is S492R, which results in loss of binding of cetuximab to EGFR. The antibody of the present disclosure binds an epitope that is different from the epitope that is recognized by Cetuximab.

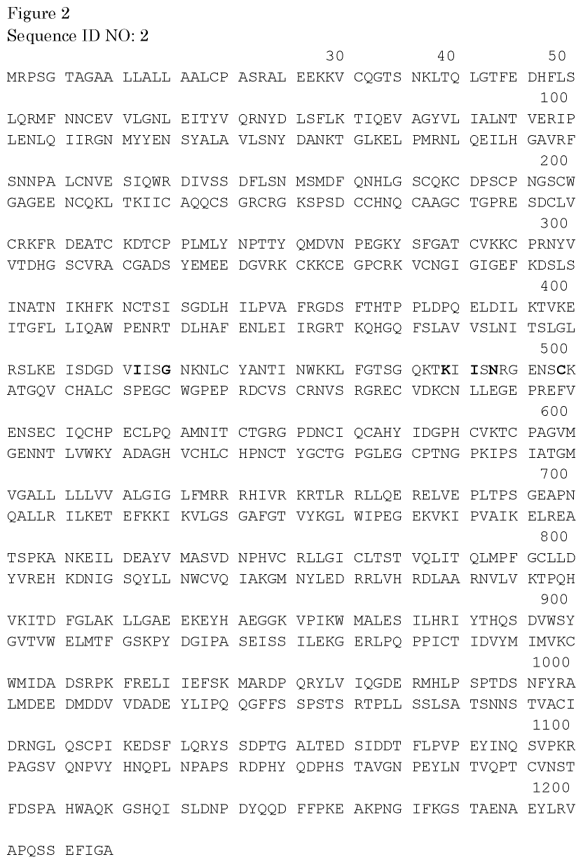

Without being bound by any theory it is believed that amino acid residues 1462; G465; K489; I491; N493; and C499 as depicted FIG. 2 are involved in binding an epitope by an antibody of the present disclosure. In certain aspects, involvement in binding is determined by observing a reduced binding of the variable domain to an EGFR with one or more of the amino acid residue substitutions selected from I462A; G465A; K489A; I491A; N493A; and C499A.

In one aspect, the variable domain that binds an epitope on an extracellular part of human EGFR is a variable domain that binds an epitope that is located within amino acid residues 420-480 of the sequence depicted in FIG. 2. In certain aspects, the binding of the variable domain to EGFR is reduced by one or more of the following amino acid residue substitutions I462A; G465A; K489A; I491A; N493A; and C499A in EGFR. In certain aspects, binding of the antibody to human EGFR interferes with the binding of EGF to the receptor. In certain aspects, the epitope on EGFR is a conformational epitope. In one aspect, the epitope is located within amino acid residues 420-480 of the sequence depicted in FIG. 2, or within 430-480 of the sequence depicted in FIG. 2. In certain aspects, said epitope is located within 438-469 of the sequence depicted in FIG. 2.

Without being bound by theory it is believed that the contact residues of the epitope, i.e. where the variable domain contacts the human EGFR are 1462; K489; I491; and N493. The amino acid residues G465 and C499 are indirectly involved in the binding of the antibody to EGFR.

In an exemplary method, CHO cells express EGFR on the cell membrane, or an alanine substitution mutant, such as a mutant comprising one or more of the substitutions selected from I462A; G465A; K489A; I491A; N493A; and C499A. A test antibody is contacted with the CHO cells and binding of the antibody to the cells compared. A test antibody binds the epitope if it binds to EGFR and to a lesser extent to an EGFR with a I462A; G465A; K489A; I491A; N493A; and C499A substitution. Comparing binding with a panel of mutants each comprising one alanine residue substitution is preferred. Such binding studies are well known in the art. Often the panel comprises single alanine substitution mutants covering essentially all amino acid residues. For EGFR the panel only needs to cover the extracellular part of the protein and a part that warrants association with the cell membrane, when cells are used.

Expression of a particular mutant can be compromised but this is detected by one or more EGFR antibodies that bind to different region(s). If expression is also reduced for these control antibodies the level or folding of the protein on the membrane is compromised for this particular mutant. Binding characteristics of the test antibody to the panel identifies whether the test antibodies exhibit reduced binding to mutants with a I462A; G465A; K489A; I491A; N493A; and C499A substitution.

In certain aspects, the disclosure provides an antibody or functional part, derivative and/or analogue thereof, that comprises a first variable domain that binds an extracellular part of EGFR, wherein the antibody is a monovalent antibody that does not comprise a second variable domain or wherein the antibody comprises EGFR binding variable domain as the only variable domain. In certain aspects, the disclosure provides an antibody or functional part, derivative and/or analogue thereof, that comprises a first variable domain that binds an extracellular part of EGFR and comprises a further second variable domain that does not bind EGFR. In certain aspects, said antibody or functional part, derivative and/or analogue thereof binds EGFR monovalently. In certain aspects, said antibody or functional part, derivative and/or analogue thereof. In certain aspects, the antibody comprises a second variable domain that binds LGR5.

In certain aspects, the disclosure provides an antibody or functional part, derivative and/or analogue thereof that comprises a first variable domain that binds an extracellular part of EGFR and a second variable domain that binds an extracellular part of LGR5.

### LGR5

The term “LGR” refers to the family of proteins known as Leucine-rich repeat-containing G-protein coupled receptors. Several members of the family are known to be involved in the WNT signaling pathway, of note LGR4, LGR5 and LGR6.

LGR5 is Leucine-Rich Repeat Containing G Protein-Coupled Receptor 5. Alternative names for the gene or protein are Leucine-Rich Repeat Containing G Protein-Coupled Receptor 5; Leucine-Rich Repeat-Containing G Protein-Coupled Receptor 5; G-Protein Coupled Receptor HG38; G-Protein Coupled Receptor 49; G-Protein Coupled Receptor 67; GPR67; GPR49; Orphan G Protein-Coupled Receptor HG38; G Protein-Coupled Receptor 49; GPR49; HG38 and FEX. A protein or antibody of the disclosure that binds LGR5, binds human LGR5. The LGR5 binding protein or antibody of the disclosure may, due to sequence and tertiary structure similarity between human and other mammalian orthologs, also bind such an ortholog but not necessarily so. Database accession numbers for the human LGR5 protein and the gene encoding it are (NC_000012.12; NT_029419.13; NC_018923.2; NP_001264155.1; NP_001264156.1; NP_003658.1). The accession numbers are primarily given to provide a further method of identification of LGR5 as a target, the actual sequence of the LGR5 protein bound may vary, for instance because of a mutation in the encoding gene such as those occurring in some cancers or the like. The LGR5 antigen binding site binds LGR5 and a variety of variants thereof, such as those expressed by some LGR5 positive tumor cells.

An antibody or a functional part, derivative and/or analogue thereof as described herein comprises a variable domain that binds an extracellular part of LGR5. In certain aspects, the second variable domain binds LGR5. In certain aspects, the LGR5 is a human LGR5. The multispecific or bispecific antibody or a functional part, derivative and/or analogue thereof as described herein comprises a variable domain that binds an extracellular part of a human epidermal growth factor (EGF receptor and in certain aspects, a variable domain that binds a human LGR5.

In certain aspects, the antibody or a functional part, derivative and/or analogue thereof as described herein comprises a variable domain that binds an extracellular part of the epidermal growth factor (EGF) receptor and interferes with the binding of EGF to the receptor and a variable domain that binds LGR5 wherein interaction of the antibody with LGR5 on an LGR5-expressing cell does not block the binding of an Rspondin (RSPO) to LGR5. Methods for determining whether an antibody blocks or does not block the binding of an Rspondin to LGR5 are described in WO2017069528, which is hereby incorporated by reference.

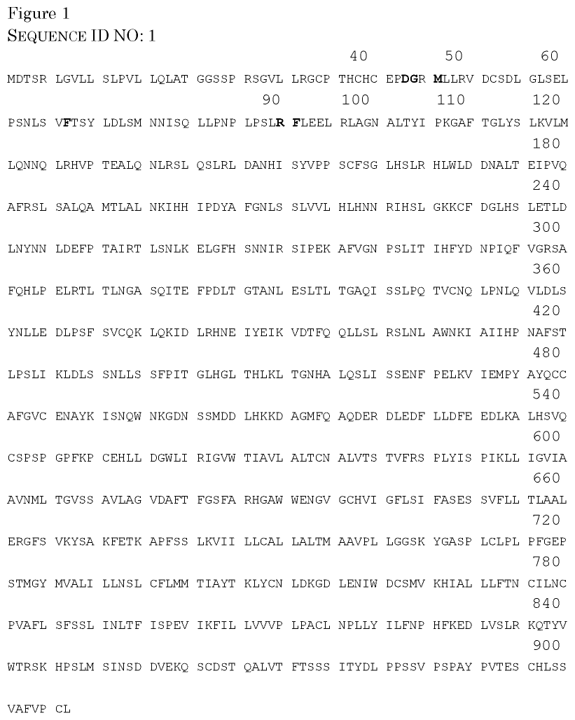

In certain aspects, the variable domain that binds an extracellular part of LGR5 binds an epitope that is located within amino acid residues 21-118 of the sequence of FIG. 1 of which amino acid residues D43; G44, M46, F67, R90, and F91 are involved in binding of the antibody to the epitope.

In certain aspects, the LGR5 variable domain is a variable domain wherein one or more of the amino acid residue substitutions in LGR5 of D43A; G44A, M46A, F67A, R90A, and F91A reduces the binding of the variable domain to LGR5.

In certain aspects, the epitope on LGR5 is a conformational epitope. In certain aspects, the epitope is located within amino acid residues 40-95 of the sequence of FIG. 1. In certain aspects, the binding of the antibody to LGR5 is reduced with one or more of the following amino acid residue substitutions D43A; G44A, M46A, F67A, R90A, and F91A.

Without being bound by theory it is believed that M46, F67, R90, and F91 of LGR5 as depicted in FIG. 1, are contact residues for a variable domain as indicated herein above, i.e. the antigen-binding site of a variable domain that binds the LGR5 epitope. That amino acid residue substitution D43A and G44A reduces the binding of an antibody can be due to the fact that these are also contact residues, however, it is also possible that these amino acid residue substitutions induce a (slight) modification of the conformation of the part of LGR5 that has one or more of the other contact residues (i.e. at positions 46, 67, 90 or 91) and that conformation change is such that antibody binding is reduced. The epitope is characterized by the mentioned amino acid substitutions. Whether an antibody binds the same epitope can be determined in various ways. In an exemplary method, CHO cells express LGR5 on the cell membrane, or an alanine substitution mutant, such as a mutant comprising one or more of the substitutions M46A, F67A, R90A, or F91A. A test antibody is contacted with the CHO cells and binding of the antibody to the cells compared. A test antibody binds the epitope if it binds to LGR5 and to a lesser extent to an LGR5 with a M46A, F67A, R90A, or F91A substitution. Comparing binding with a panel of mutants each comprising one alanine residue substitution is preferred. Such binding studies are well known in the art. Often the panel comprises single alanine substitution mutants covering essentially all amino acid residues. For LGR5 the panel only needs to cover the extracellular part of the protein and a part that warrants association with the cell membrane of course, when cells are used. Expression of a particular mutant can be compromised but this is easily detected by one or more LGR5 antibodies that bind to different region(s). If expression is also reduced for these control antibodies the level or folding of the protein on the membrane is compromised for this particular mutant. Binding characteristics of the test antibody to the panel identifies whether the test antibodies exhibit reduced binding to mutants with a M46A, F67A, R90A, or F91A substitution and thus whether the test antibody is an antibody of the disclosure. Reduced binding to mutants with a M46A, F67A, R90A, or F91A substitution also identifies the epitope to be located within amino acid residues 21-118 of the sequence of FIG. 1. In certain aspects, the panel includes a D43A substitution mutant; a G44A substitution mutant of both. The antibody with the VH sequence of the VH of MF5816 exhibits reduced binding to these substitution mutants.

Where herein accession numbers or alternative names of proteins/genes are given, they are primarily given to provide a further method of identification of the mentioned protein as a target, the actual sequence of the target protein bound by an antibody of the disclosure may vary, for instance because of a mutation and/or alternative splicing in the encoding gene such as those occurring in some cancers or the like. The target protein is bound by the antibody as long as the epitope is present in the protein and the epitope is accessible to the antibody.

### Antibodies Binding EGFR and LGR5

The disclosure further provides an antibody with a variable domain that binds an extracellular part of EGFR and a variable domain that binds an extracellular part of LGR5 wherein the LGR5 variable domain binds an epitope on LGR5 that is located within amino acid residues 21-118 of the sequence of FIG. 1.

Suitable variable domains that bind an extracellular part of EGFR and suitable variable domains that bind an extracellular part of LGR5 are disclosed herein. In certain aspects, the first variable domain comprises at least the CDR3 sequence, or at least the CDR1, CDR2 and CDR3 sequences of an EGFR specific heavy chain variable region selected from the group consisting of MF3370; MF3755; MF4280 or MF4289 as depicted in FIG. 3. In certain aspects, the second variable domain comprises at least the CDR3 sequence, or at least the CDR1, CDR2 and CDR3 sequences of an LGR5 specific heavy chain variable region selected from the group consisting of MF5790; MF5803; MF5805; MF5808; MF5809; MF5814; MF5816; MF5817; or MF5818 as depicted in FIG. 3.

In certain aspects, the variable domain that binds human EGFR, is a variable domain with a heavy chain variable region that comprises at least the CDR3 sequence of the VH of MF3755 as depicted in FIG. 3 or a CDR3 sequence that differs in at most three, or in at most two, or in no more than one amino acid from a CDR3 sequence of the VH of MF3755 as depicted in FIG. 3.

In certain aspects, the variable domain that binds human EGFR, is a variable domain with a heavy chain variable region that comprises at least the CDR1, CDR2 and CDR3 sequences of the VH of MF3755 as depicted in FIG. 3; or the CDR1, CDR2 and CDR3 sequences of the VH of MF3755 as depicted in FIG. 3 with at most three, or at most two, or at most one amino acid substitutions.

In certain aspects, the variable domain that binds human EGFR, is a variable domain with a heavy chain variable region that comprises the sequence of the VH chain of MF3755 as depicted in FIG. 3; or the amino acid sequence of the VH chain of MF3755 depicted in FIG. 3 having at most 15 (or in certain aspects 1, 2, 3, 4, 5, 6, 7, 8, 9 or 10, or in certain aspects 1, 2, 3, 4 or 5) amino acid insertions, deletions, substitutions or a combination thereof with respect to the VH chain of MF3755.

In certain aspects, the disclosure provides an antibody comprising a variable domain that binds an extracellular part of EGFR and a variable domain that binds an extracellular part of LGR5, wherein a heavy chain variable region of said variable domain comprises at least the CDR3 sequence of an EGFR specific heavy chain variable region selected from the group consisting of MF3370; MF3755; MF4280 or MF4289 as depicted in FIG. 3 or wherein a heavy chain variable region of said variable domain comprises a heavy chain CDR3 sequence that differs in at most three, or in at most two, or in no more than one amino acid from a CDR3 sequence of a VH selected from the group consisting of MF3370; MF3755; MF4280 or MF4289 as depicted in FIG. 3. In certain aspects, said variable domain comprises a heavy chain variable region comprising at least the CDR3 sequence of MF3370; MF3755; MF4280 or MF4289 as depicted in FIG. 3.

In certain aspects, said variable domain comprises a heavy chain variable region comprising at least the CDR1, CDR2 and CDR3 sequences of an EGFR specific heavy chain variable region selected from the group consisting of MF3370; MF3755; MF4280 or MF4289 as depicted in FIG. 3, or heavy chain variable region comprising at least CDR1, CDR2 and CDR3 sequences that differ in at most three, or in at most two, or in at most one amino acid from the CDR1, CDR2 and CDR3 sequences of an EGFR specific heavy chain variable region selected from the group consisting of MF3370; MF3755; MF4280 or MF4289 as depicted in FIG. 3. In certain aspects, said variable domain comprises a heavy chain variable region comprising at least the CDR1, CDR2 and CDR3 sequences of MF3370; MF3755; MF4280 or MF4289 as depicted in FIG. 3. In certain aspects, the heavy chain variable region is MF3755. In certain aspects, the heavy chain variable region is MF4280.

In certain aspects, the disclosure provides an antibody comprising a variable domain that binds an extracellular part of EGFR and a variable domain that binds an extracellular part of LGR5, wherein the variable domain that binds an extracellular part of EGFR is a heavy chain variable region that comprises the amino acid sequence of the VH chain of MF3370; MF3755; MF4280 or MF4289 as depicted in FIG. 3, or the amino acid sequence of the VH chain of MF3370; MF3755; MF4280 or MF4289 depicted in FIG. 3 having at most 15, or 1, 2, 3, 4, 5, 6, 7, 8, 9, or 10 amino acid insertions, deletions, substitutions, or a combination thereof with respect to the VH chain of MF3370; MF3755; MF4280 or MF4289.

In certain aspects, the antibody comprising a variable domain that binds an extracellular part of EGFR and a variable domain that binds an extracellular part of LGR5, wherein the EGFR binding variable domains has a CDR3, a CDR1, CDR2, and CDR3 and/or a VH sequence as indicated herein above, has a variable domain that binds LGR5 that comprises at least the CDR3 sequence of an LGR5 specific heavy chain variable region selected from the group consisting of MF5790; MF5803; MF5805; MF5808; MF5809; MF5814; MF5816; MF5817; or MF5818 as depicted in FIG. 3 or a heavy chain CDR3 sequence that differs in at most three, or in at most two, or in no more than one amino acid from a CDR3 sequence of a VH selected from the group consisting of MF5790; MF5803; MF5805; MF5808; MF5809; MF5814; MF5816; MF5817; or MF5818 as depicted in FIG. 3. In certain aspects, said variable domain comprises a heavy chain variable region comprising at least the CDR3 sequence of MF5790; MF5803; MF5805; MF5808; MF5809; MF5814; MF5816; MF5817; or MF5818 as depicted in FIG. 3.

In certain aspects, the LGR5 binding variable domain comprises a heavy chain variable region comprising at least the CDR1, CDR2 and CDR3 sequences of an LGR5 specific heavy chain variable region selected from the group consisting of MF5790; MF5803; MF5805; MF5808; MF5809; MF5814; MF5816; MF5817; or MF5818 as depicted in FIG. 3, or heavy chain CDR1, CDR2 and CDR3 sequences that differ in at most three, or in at most two, or in at most one amino acid from the CDR1, CDR2 and CDR3 sequences of LGR5 specific heavy chain variable region selected from the group consisting of MF5790; MF5803; MF5805; MF5808; MF5809; MF5814; MF5816; MF5817; or MF5818 as depicted in FIG. 3. In certain aspects, said variable domain comprises a heavy chain variable region comprising at least the CDR1, CDR2 and CDR3 sequences of MF5790; MF5803; MF5805; MF5808; MF5809; MF5814; MF5816; MF5817; or MF5818 as depicted in FIG. 3. In certain aspects, the heavy chain variable region is MF5790; MF5803; MF5814; MF5816; MF5817; or MF5818. In certain aspects, the heavy chain variable region is MF5790; MF5814; MF5816; or MF5818. In certain aspects, the heavy chain variable region is MF5814, MF5818 or MF5816. In certain aspects, the heavy chain variable region is MF5816. In certain aspects, the heavy chain variable region is MF5818.

In certain aspects, the disclosure provides an antibody comprising a variable domain that binds an extracellular part of EGFR and a variable domain that binds an extracellular part of LGR5, wherein the variable domain that binds an extracellular part of LGR5 is a heavy chain variable region that comprises the amino acid sequence of the VH chain of MF5790; MF5803; MF5805; MF5808; MF5809; MF5814; MF5816; MF5817; or MF5818 as depicted in FIG. 3, or the amino acid sequence of the VH chain of MF5790; MF5803; MF5805; MF5808; MF5809; MF5814; MF5816; MF5817; or MF5818 depicted in FIG. 3 having at most 15, or 1, 2, 3, 4, 5, 6, 7, 8, 9, or 10 amino acid insertions, deletions, substitutions, or a combination thereof with respect to the VH chain of MF5790; MF5803; MF5805; MF5808; MF5809; MF5814; MF5816; MF5817; or MF5818.

It has been shown that the antibodies comprising one or more variable domains with a heavy chain variable region MF3755 or one or more CDRs thereof have a better effectivity when used to inhibit growth of an EGFR ligand responsive cancer or cell. In the context of bispecific or multispecific antibodies, an arm of the antibody comprising a variable domain with a heavy chain variable region MF3755 or one or more CDRs thereof combines well with an arm comprising a variable domain with a heavy chain variable region MF5818 or one or more CDRs thereof.

It has been shown that antibodies comprising one or more variable domains with a heavy chain variable region MF3755 or one or more CDRs thereof have a better effectivity when used to inhibit growth of an EGFR ligand responsive cancer or cell. In the context of bispecific or multispecific antibodies, an arm of the antibody comprising a variable domain with a heavy chain variable region MF3755 or one or more CDRs thereof combines well with an arm comprising a variable domain with a heavy chain variable region MF5816 or one or more CDRs thereof.

VH chains of variable domains that bind EGFR or LGR5 can have one or more amino acid substitutions with respect to the sequence depicted in FIG. 3. In certain aspects, a VH chain has an amino acid sequence of an EGFR or LGR5 VH of FIG. 3, having at most 15, or 1, 2, 3, 4, 5, 6, 7, 8, 9 or 10 and, in certain aspects, having 1, 2, 3, 4 or amino acid insertions, deletions, substitutions or a combination thereof with respect to the VH chain sequence of FIG. 3.

CDR sequences can have one or more amino acid residue substitutions with respect to a CDR sequence in the figures. Such one or more substitutions are for instance made for optimization purposes, such as to improve binding strength or the stability of the antibody. Optimization is for instance performed by mutagenesis procedures where the stability and/or binding affinity of the resulting antibodies are for instance tested and an improved EGFR specific CDR sequence or LGR5 specific CDR sequence is selected. A skilled person is well capable of generating antibody variants comprising at least one altered CDR sequence according to the disclosure. For instance, conservative amino acid substitution may be applied. Examples of conservative amino acid substitution include the substitution of one hydrophobic residue such as isoleucine, valine, leucine or methionine for another hydrophobic residue, and the substitution of one polar residue for another polar residue, such as the substitution of arginine for lysine, glutamic acid for aspartic acid, or glutamine for asparagine.

In certain aspects, the mentioned at most 15 (or in certain aspects, 1, 2, 3, 4, 5, 6, 7, 8, 9 or 10 or in certain aspects 1, 2, 3, 4 or 5) amino acid substitutions in a VH or VL as specified herein are the conservative amino acid substitutions. In certain aspects, the amino acid insertions, deletions and substitutions or a combination thereof in a VH or VL as specified herein are not present in the CDR3 region. In certain aspects, the mentioned amino acid insertions, deletions and substitutions are also not present in the CDR1 and CDR2 regions. In certain aspects, the mentioned amino acid insertions, deletions and substitutions are also not present in the FR4 region.

In certain aspects, said insertions, deletions, substitutions or a combination thereof are not present in the CDR3 region of the VH chain, in certain aspects, not present in the CDR1, CDR2 or CDR3 region of the VH chain and in certain aspects, not in the FR4 region.

In certain aspects, said insertions, deletions, substitutions or a combination thereof are not present in the CDR1, CDR2 and CDR3 region of the VH chain.

In certain aspects, the disclosure provides an antibody comprising a variable domain that binds an extracellular part of EGFR and in certain aspects a variable domain that binds an extracellular part of LGR5 which comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect to said VH; and
wherein the VH chain of the variable domain that binds LGR5 comprises
the amino acid sequence of VH chain MF5790 as depicted in FIG. 3; or
the amino acid sequence of VH chain MF5790 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect to said VH.

In certain aspects, the disclosure provides an antibody comprising a variable domain that binds an extracellular part of EGFR and a variable domain that, in certain aspects, binds an extracellular part of LGR5 comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect to said VH; and
wherein the VH chain of the variable domain that binds LGR5 comprises
the amino acid sequence of VH chain MF5803 as depicted in FIG. 3; or
the amino acid sequence of VH chain MF5803 as depicted in FIG. 3 having at most 15 (or in certain aspects, 1, 2, 3, 4, 5, 6, 7, 8, 9 or 10, or in certain aspects 1, 2,47 3, 4 or 5) amino acid insertions, deletions, substitutions or a combination thereof with respect to said VH.

In certain aspects, the disclosure provides an antibody comprising a variable domain that binds an extracellular part of EGFR and a variable domain that, in certain aspects, binds an extracellular part of LGR5 comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect to said VH; and
wherein the VH chain of the variable domain that binds LGR5 comprises
the amino acid sequence of VH chain MF5814 as depicted in FIG. 3; or
the amino acid sequence of VH chain MF5814 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect to said VH.

In certain aspects, the disclosure provides an antibody comprising a variable domain that binds an extracellular part of EGFR and a variable domain that, in certain aspects, binds an extracellular part of LGR5 comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect to said VH; and
wherein the VH chain of the variable domain that binds LGR5 comprises
the amino acid sequence of VH chain MF5816 as depicted in FIG. 3; or
the amino acid sequence of VH chain MF5816 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect to said VH.

In certain aspects, the disclosure provides an antibody comprising a variable domain that binds an extracellular part of EGFR and a variable domain that, in certain aspects, binds an extracellular part of LGR5 comprises
the amino acid sequence of VH chain MF3755 as depicted in FIG. 3; or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect to said VH; and
wherein the VH chain of the variable domain that binds LGR5 comprises
the amino acid sequence of VH chain MF5817 as depicted in FIG. 3; or
the amino acid sequence of VH chain MF5817 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect to said VH.

In certain aspects, the disclosure provides an antibody comprising a variable domain that binds an extracellular part of EGFR and a variable domain that, in certain aspects, binds an extracellular part of LGR5 comprises
the amino acid sequence of VH chain MF3755 as depicted in FIG. 3 or
the amino acid sequence of VH chain MF3755 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect to said VH; and
wherein the VH chain of the variable domain that binds LGR5 comprises
the amino acid sequence of VH chain MF5818 as depicted in FIG. 3; or
the amino acid sequence of VH chain MF5818 as depicted in FIG. 3 having at most 15 (or in certain aspects, 1, 2, 3, 4, 5, 6, 7, 8, 9 or 10, or in certain aspects 1, 2, 3, 4 or 5) amino acid insertions, deletions, substitutions or a combination thereof with respect to said VH.

In certain aspects, the antibody is petosemtamab (cf. WHO Drug Information, Recommended INN: List 83. Vol. 34, No. 1, 2020, Pg75-77). In certain aspects, the functional part, derivative and/or analogue thereof is a functional part, derivative and/or analogue thereof of petosemtamab. In certain aspects, the EGFR/LGR5 antibody of the present disclosure is or comprises petosemtamab.

In certain aspects, the variable domain that binds an extracellular part of EGFR comprises a heavy chain variable region comprising the CDR1, CDR2 and CDR3 sequences of a variable region selected from the group consisting of MF3370; MF3755; MF4280 or MF4289 as depicted in FIG. 3 and wherein the variable domain that binds an extracellular part of LGR5 comprises a heavy chain variable region comprising the CDR1, CDR2 and CDR3 sequences of a variable region selected from the group consisting of MF5790; MF5803; MF5805; MF5808; MF5809; MF5814; MF5816; MF5817; or MF5818 as depicted in FIG. 3.

In certain aspects, the variable domain that binds an extracellular part of EGFR comprises a heavy chain variable region comprising the CDR1, CDR2 and CDR3 sequences of the variable region of MF3755 as depicted in FIG. 3 and wherein the variable domain that binds an extracellular part of LGR5 comprises a heavy chain variable region comprising the CDR1, CDR2 and CDR3 sequences of the variable region of MF5816 as depicted in FIG. 3.

In certain aspects, a VH chain of the variable domain that binds EGFR comprises the amino acid sequence of VH chain MF3370; MF3755; MF4280 or MF4289 as depicted in FIG. 3; or the amino acid sequence of VH chain MF3370; MF3755; MF4280 or MF4289 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH; and wherein a VH chain of the variable domain that binds LGR5 comprises the amino acid sequence of VH chain MF5790; MF5803; MF5805; MF5808; MF5809; MF5814; MF5816; MF5817; or MF5818 as depicted in FIG. 3; or the amino acid sequence of VH chain MF5790; MF5803; MF5805; MF5808; MF5809; MF5814; MF5816; MF5817; or MF5818 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH.

In certain aspects, a VH chain of the variable domain that binds EGFR comprises the amino acid sequence of VH chain MF3755 as depicted in FIG. 3; or the amino acid sequence of VH chain MF3755 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH; and wherein a VH chain of the variable domain that binds LGR5 comprises the amino acid sequence of VH chain MF5816 as depicted in FIG. 3; or the amino acid sequence of VH chain MF5816 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH.

In certain aspects, a VH chain of the variable domain that binds EGFR comprises the amino acid sequence of VH chain MF3370; MF3755; MF4280 or MF4289 as depicted in FIG. 3; and wherein a VH chain of the variable domain that binds LGR5 comprises the amino acid sequence of VH chain MF5790; MF5803; MF5805; MF5808; MF5809; MF5814; MF5816; MF5817; or MF5818 as depicted in FIG. 3.

In certain aspects, a VH chain of the variable domain that binds EGFR comprises the amino acid sequence of VH chain MF3755 as depicted in FIG. 3; and wherein a VH chain of the variable domain that binds LGR5 comprises the amino acid sequence of VH chain MF5816 as depicted in FIG. 3.

In certain aspects, both said variable domains that bind EGFR and that bind LGR5 comprise the CDR1, CDR2 and CDR3 regions of the light chain variable region as depicted in FIG. 4b.

In certain aspects, both said variable domains that bind EGFR and that bind LGR5 comprise the light chain variable region as depicted in FIG. 4b, which variable light chain region comprises from 0 to 10 amino acid insertions, deletions, substitutions, additions or a combination thereof, wherein the amino acid insertions, deletions and substitutions are not present in the CDR1, CDR2 and CDR3 light chain variable regions.

Additional variants of the disclosed amino acid sequences which retain EGFR or LGR5 binding can be obtained, for example, from phage display libraries which contain the rearranged human IGKV1-39/IGKJ1 VL region (De Kruif et al. Biotechnol Bioeng. 2010 (106)741-50), and a collection of VH regions incorporating amino acid substitutions into the amino acid sequence of an EGFR or LGR5 VH region disclosed herein, as previously described (e.g., WO2017/069628). Phages encoding Fab regions which bind EGFR or LGR5 may be selected and analyzed by flow cytometry, and sequenced to identify variants with amino acid substitutions, insertions, deletions or additions which retain antigen binding.

In certain aspects, the light chain variable regions of the VH/VL EGFR and LGR5 variable domains of an EGFR/LGR5 antibody of the present disclosure may be the same or different. In certain aspects, the VL region of the VH/VL EGFR variable domain of said EGFR/LGR5 antibody is similar to the VL region of the VH/VL LGR5 variable domain. In certain aspects, VL regions in the first and second VH/VL variable domains are identical.

In certain aspects, the light chain variable region of one or both VH/VL variable domains of said EGFR/LGR5 antibody comprises a common light chain variable region. In certain aspects, the common light chain variable region of one or both VH/VL variable domains comprises a germline IgVκ1-39 variable region V-segment. In certain aspects, the light chain variable region of one or both VH/VL variable domains comprises the kappa light chain V-segment IgVκ1-39*01. IgVκ1-39 is short for Immunoglobulin Variable Kappa 1-39 Gene. The gene is also known as Immunoglobulin Kappa Variable 1-39; IGKV139; IGKV1-39. External Ids for the gene are HGNC: 5740; Entrez Gene: 28930; Ensembl: ENSG00000242371. The amino acid sequence for a suitable V-region is provided in FIG. 4. The V-region can be combined with one of five J-regions. In certain aspects, the J-regions are jk1 and jk5, and the joined sequences are indicated as IGKV1-39/jk1 and IGKV1-39/jk5; alternative names are IgVκ1-39*01/IGJκ1*01 or IgVκ1-39*01/IGJκ5*01 (nomenclature according to the IMGT database worldwide web at imgt.org). In certain aspects, the light chain variable region of one or both VH/VL variable domains comprises the kappa light chain IgVκ1-39*01/IGJκ1*01 or IgVκ1-39*01/IGJκ1*05 (described in FIG. 4).

In certain aspects, the light chain variable region of FIG. 4d) comprises LCDR1, LCDR2 and LCDR sequences. Such sequences can be determined or annotated by the skilled person using for instance an annotation system like IMGT, Chothia, Kabat or other suitable annotation systems.

CDRs and framework regions of antibody heavy and light chains have been described and defined in the art using a number of different systems, including for instance Kabat (see Sequences of Proteins of Immunological Interest (National Institute of Health, Bethesda, Md., 1987 and 1991); Kabat et al., J. Biol. Chem. 252:6609-6616 (1977)), IMGT (discussed in Giudicelli et al., Nucleic Acids Res. 25: 206-21 1 1997), Chothia (Chothia and Lesk J. Mol. Biol. 196: 901-917, 1987; Chothia et al., Nature 342: 877-883, 1989; Al-Lazikani et al., J. Mol. Biol. 273: 927-948, 1997), and the nomenclatures of Honnegher and Pluckthun (Honnegher and Pluckthun, J. Mol. Biol. 309: 657-670, 2001), MacCallum (MacCallum et al., J. Mol. Biol. 262:732-745 (1996); Abhinandan and Martin, Mol. Immunol., 45: 3832-3839 (2008)), and Lefranc (Lefranc M. P. et al., Dev. Comp. Immunol., 27: 55-77 (2003)). In general, it is irrelevant which numbering system is used, as an antibody exhibits its properties regardless of the numbering system used. When the amino acid sequence of a variable region is given, a skilled person can readily determine its CDRs based on of the above indicated different numbering systems. Thus, the present disclosure encompasses defining the CDRs in accordance with each numbering system available to a skilled person. In particular, the present disclosure encompasses defining the CDRs in accordance with the numbering systems of Kabat, IMGT, and Chothia. In certain aspects, the heavy chain CDRs are as defined using Kabat and the light chain CDRs as defined using IMGT. Amino acids in the constant regions are indicated according to the EU numbering system.

In certain aspects, said light chain variable region is or comprises one or both VH/VL variable domains that are present in an EGFR binding antibody of the present disclosure and comprise LCDR1, LCDR2 and LCDR sequences. Such sequences can be determined or annotated by the skilled person using for instance an annotation system like IMGT, Chothia, Kabat or other suitable annotation systems.

In certain aspects, the light chain variable region of one or both VH/VL variable domains of a bispecific antibody of the present disclosure comprises an LCDR1 comprising the amino acid sequence QSISSY (described in FIG. 4), an LCDR2 comprising the amino acid sequence AAS (described in FIG. 4), and an LCDR3 comprising the amino acid sequence QQSYSTPPT (described in FIG. 4) (i.e., the CDRs of IGKV1-39 according to IMGT). In certain aspects, the light chain variable region of one or both VH/VL variable domains of an EGFR/LGR5 binding antibody of the present disclosure comprises an LCDR1 comprising the amino acid sequence QSISSY (described in FIG. 4), an LCDR2 comprising the amino acid sequence AAS (described in FIG. 4), and an LCDR3 comprising the amino acid sequence QQSYSTPPT (described in FIG. 4).

In certain aspects, the light chain variable region of one or both VH/VL variable domains of an EGFR/LGR5 bispecific antibody of the present disclosure comprises an LCDR1 comprising the amino acid sequence QSISSY (described in FIG. 4), an LCDR2 comprising the amino acid sequence AAS (described in FIG. 4), and an LCDR3 comprising the amino acid sequence QQSYSTPPT (described in FIG. 4) (i.e., the CDRs of IGKV1-39 according to IMGT). In certain aspects, the light chain variable region of one or both VH/VL variable domains of said EGFR/LGR5 antibody comprises an LCDR1 comprising the amino acid sequence QSISSY (described in FIG. 4), an LCDR2 comprising the amino acid sequence AAS (described in FIG. 4), and an LCDR3 comprising the amino acid sequence QQSYSTPPT (described in FIG. 4).

In certain aspects, one or both VH/VL variable domains of the EGFR/LGR5 antibody comprise a light chain variable region comprising an amino acid sequence that is at least 90%, in certain aspects at least 95%, in certain aspects at least 97%, in certain aspects at least 98%, in certain aspects at least 99% identical or in certain aspects 100% identical to the amino acid sequence as set forth in FIG. 4.

For example, the light chain variable region of one or both VH/VL variable domains of the EGFR/LGR5 antibody can have from 0 to 10, or in certain aspects from 0 to 5 amino acid insertions, deletions, substitutions, additions or a combination thereof with respect to the sequence in FIG. 4. In certain aspects, the light chain variable region of one or both VH/VL variable domains of the EGFR/LGR5 antibody comprises from 0 to 9, from 0 to 8, from 0 to 7, from 0 to 6, from 0 to 5, from 0 to 4, in certain aspects from 0 to 3, in certain aspects from 0 to 2, in certain aspects from 0 to 1 and in certain aspects 0 amino acid insertions, deletions, substitutions, additions with respect to the indicated amino acid sequence, or a combination thereof.

Also, the light chain variable region of one or both VH/VL variable domains of the EGFR/LGR5 antibody may comprise the amino acid sequence of a sequence as depicted in FIG. 4. In certain aspects, both VH/VL variable domains of the EGFR/LGR5 antibody comprise identical VL regions. In certain aspects, the VL of both VH/VL variable domains of the EGFR/LGR5 bispecific antibody comprises the amino acid sequence set forth in FIG. 4. In certain aspects, the VL of both VH/VL variable domains of the EGFR/LGR5 bispecific antibody comprises the amino acid sequence set forth in FIG. 4.

In certain aspects, the antibody or functional part, derivative and/or analogue thereof that comprises a variable domain that binds an extracellular part of EGFR is a bispecific antibody having two variable domains, one that binds EGFR and another that binds LGR5 as described herein. Said antibody or functional part, derivative and/or analogue thereof, can be provided in a number of formats. Many different formats of bispecific antibodies are known in the art, and have been reviewed by Kontermann (Drug Discov Today, 2015 July; 20(7):838-47; MAbs, 2012 March-April; 4(2):182-97) and in Spiess et al., (Alternative molecular formats and therapeutic applications for bispecific antibodies. Mol. Immunol. (2015) http: //dx.doi.org/10.1016/j.molimm.2015.01.003), which are each incorporated herein by reference. For example, bispecific antibody formats that are not classical antibodies with two VH/VL combinations, have at least a variable domain comprising a heavy chain variable region and a light chain variable region. This variable domain may be linked to a single chain Fv-fragment, monobody, a VH and a Fab-fragment that provides the second binding activity.

In certain aspects, said antibody or functional part, derivative and/or analogue thereof is a bispecific antibody of the human IgG subclass (e.g., for instance IgG1, IgG2, IgG3, IgG4). In certain aspects, said antibody is of the human IgG1 subclass. Full length IgG antibodies are preferred because of their favorable half-life and for reasons of low immunogenicity. Accordingly, said bispecific antibody is in certain aspects, a full length IgG molecule. In certain aspects, said bispecific antibody is a full length IgG1 molecule. Accordingly, in certain aspects, said bispecific antibody comprises a fragment crystallizable (Fc). In certain aspects, the Fc of the EGFR/LGR5 bispecific antibody is comprised of a human constant region. A constant region or Fc of the EGFR/LGR5 bispecific antibody may contain one or more, or not more than 10, or not more than 5 amino-acid differences with a constant region of a naturally occurring human antibody. For example, each Fab-arm of the bispecific antibodies may further include an Fc-region comprising modifications promoting the formation of the bispecific antibody, promoting stability and/or other features described herein.

### EGFR Signal Inhibition

In certain aspects, an antibody or a functional part, derivative and/or analogue thereof as described herein interferes with the binding of a ligand for EGFR to EGFR. The term “interferes with binding” as used herein means that binding of the antibody or a functional part, derivative and/or analogue thereof to the EGFR competes with the ligand for binding to EGF receptor. The antibody or a functional part, derivative and/or analogue thereof may diminish ligand binding, displace ligand when this is already bound to the EGF receptor or it may, for instance through steric hindrance, at least partially prevent that ligand can bind to the EGF receptor.

In certain aspects, an EGFR antibody as disclosed herein inhibits respectively EGFR ligand-induced signaling, measured as ligand-induced growth of BxPC3 cells (ATCC CRL-1687) or BxPC3-luc2 cells (Perkin Elmer 125058) or ligand-induced cell death of A431 cells (ATCC CRL-1555). EGFR can bind a number of ligands and stimulate growth of the mentioned BxPC3 cells or BxPC3-luc2 cells. In the presence of an EGFR ligand the growth of BxPC3 or BxPC3-luc2 cells is stimulated. EGFR ligand-induced growth of BxPC3 cells can be measured by comparing the growth of the cells in the absence and presence of the ligand. In certain aspects, the EGFR ligand for measuring EGFR ligand-induced growth of BxPC3 or BxPC3-luc2 cells is EGF. In certain aspects, the ligand-induced growth is measured using saturating amounts of ligand. In certain aspects, EGF is used in an amount of 100 ng/ml of culture medium. In certain aspects, said EGF is the EGF from R&D systems, cat. nr. 396-HB and 236-EG (see also WO2017/069628; which is incorporated by reference herein).

In certain aspects, an EGFR antibody as disclosed herein inhibits EGFR ligand induced growth of BxPC3 cells (ATCC CRL-1687) or BxPC3-luc2 cells (Perkin Elmer 125058). EGFR can bind a number of ligands and stimulate growth of the mentioned BxPC3 cells or BxPC3-luc2 cells. In the presence of a ligand the growth of BxPC3 or BxPC3-luc2 cells is stimulated. EGFR ligand-induced growth of BxPC3 cells can be measured by comparing the growth of the cells in the absence and presence of the ligand. In certain aspects, the EGFR ligand for measuring EGFR ligand-induced growth of BxPC3 or BxPC3-luc2 cells is EGF. In certain aspects, the ligand-induced growth is measured using saturating amounts of ligand. In certain aspects, EGF is used in an amount of 100 ng/ml of culture medium. In certain aspects, EGF is the EGF of R&D systems, cat. nr. 396-HB and 236-EG (see also WO2017/069628; which is incorporated by reference herein).

For the avoidance of doubt the reference to the growth of a cell as used herein refers to a change in the number of cells. Inhibition of growth refers to a reduction in the number of cells that would otherwise have been obtained. Increase in growth refers to an increase in the number of cells that would otherwise have been obtained. The growth of a cell typically refers to the proliferation of the cell.

Whether an antibody as described herein inhibits signaling or inhibits growth in a multispecific format is in certain aspects determined by the methods as described herein above using a monospecific monovalent or monospecific bivalent version of the antibody. In certain aspects, such an antibody has binding sites for the receptor of which signaling is to be determined. A monospecific monovalent antibody can have a variable domain with an irrelevant binding specificity such as tetanus toxoid specificity. In certain aspects, said antibody is a bivalent monospecific antibody wherein the antigen binding variable domains consist of variable domains that bind the EGF-receptor family member.

Nucleic acids and cells Bispecific antibodies are typically produced by cells that express nucleic acid(s) encoding the antibody. Accordingly, in certain aspects, the bispecific EGFR/LGR5 antibodies disclosed herein are produced by providing a cell comprising one or more nucleic acids that encode the heavy and light chain variable regions and constant regions of the bispecific EGFR/LGR5 antibody. In certain aspects, the cell is an animal cell, such as a mammal cell, or a primate cell and in certain aspects a human cell. A suitable cell is any cell capable of comprising and producing the EGFR/LGR5 bispecific antibody.

Suitable cells for antibody production are known in the art and include a hybridoma cell, a Chinese hamster ovary (CHO) cell, an NS0 cell or a PER-C6 cell. Various institutions and companies have developed cell lines for the large scale production of antibodies, for instance for clinical use. Non-limiting examples of such cell lines are CHO cells, NSwq cells or PER.C6 cells. In certain aspects, said cell is a human cell. In certain aspects a cell is transformed by an adenovirus E l region or a functional equivalent thereof. An example of such a cell line is the PER.C6 cell line or equivalent thereof. In a certain aspect, said cell is a CHO cell or a variant thereof. In certain aspects, the variant makes use of a glutamine synthetase (GS) vector system for expression of an antibody. In certain aspects, the cell is a CHO cell.

In certain aspects, the cell expresses the different light and heavy chains that make up the EGFR/LGR5 bispecific antibody. In certain aspects, the cell expresses two different heavy chains and at least one light chain. In certain aspects, the cell expresses a “common light chain” as described herein to reduce the number of different antibody species (combinations of different heavy and light chains). For example, the respective VH regions are cloned into expression vectors using methods known in the art for production of bispecific IgG (WO2013/157954; incorporated herein by reference), in conjunction with the rearranged human IGKV1-39/IGKJ1 (huVκ1-39) light chain, previously shown to be able to pair with more than one heavy chain thereby giving rise to antibodies with diverse specificities, which facilitates the generation of bispecific molecules (De Kruif et al. J. Mol. Biol. 2009 (387) 548 58; WO2009/157771).

An antibody producing cell that expresses a common light chain and equal amounts of the two heavy chains typically produces 50% bispecific antibody and 25% of each of the monospecific antibodies (i.e. having identical heavy light chain combinations). Several methods have been published to favor the production of the bispecific antibody over the production of the respective monospecific antibodies. Such is typically achieved by modifying the constant region of the heavy chains such that they favor heterodimerization (i.e. dimerization with the heavy chain of the other heavy/light chain combination) over homodimerization. In certain aspects, the bispecific antibody of the disclosure comprises two different immunoglobulin heavy chains with compatible heterodimerization domains. Various compatible heterodimerization domains have been described in the art. In certain aspects, the compatible heterodimerization domains are compatible immunoglobulin heavy chain CH3 heterodimerization domains. The art describes various ways in which such hetero-dimerization of heavy chains can be achieved.

One preferred method for producing the EGFR/LGR5 bispecific antibody is disclosed in U.S. Pat. Nos. 9,248,181 and 9,358,286. Specifically, preferred mutations to produce essentially only bispecific full length IgG molecules are the amino acid substitutions L351K and T366K (EU numbering) in the first CH3 domain (the ‘KK-variant’ heavy chain) and the amino acid substitutions L351D and L368E in the second CH3 domain (the ‘DE-variant’ heavy chain), or vice versa. As previously described, the DE-variant and KK-variant preferentially pair to form heterodimers (so-called ‘DEKK’ bispecific molecules). Homodimerization of DE-variant heavy chains (DEDE homodimers) or KK-variant heavy chains (KKKK homodimers) hardly occurs due to strong repulsion between the charged residues in the CH3-CH3 interface between identical heavy chains.

Accordingly, in certain aspects, the heavy chain/light chain combination that comprises the variable domain that binds EGFR, comprises a DE variant of the heavy chain. In certain aspects, the heavy chain/light chain combination that comprises the variable domain that binds LGR5 comprises a KK variant of the heavy chain.

A candidate EGFR/LGR5 IgG bispecific antibody can be tested for binding using any suitable assay. For example, binding to membrane-expressed EGFR or LGR5 on CHO cells can be assessed by flow cytometry (according to the FACS procedure as previously described in WO2017/069628). In certain aspects, the binding of a candidate EGFR/LGR5 bispecific antibody to LGR5 on CHO cells is demonstrated by flow cytometry, performed according to standard procedures known in the art. Binding to the CHO cells is compared with CHO cells that have not been transfected with expression cassettes for EGFR and/or LGR5. The binding of the candidate bispecific IgG1 to EGFR is determined using CHO cells transfected with an EGFR expression construct; a LGR5 monospecific antibody and an EGFR monospecific antibody, as well as an irrelevant IgG1 isotype control mAb are included in the assay as controls (e.g., an antibody which binds LGR5 and another antigen such as tetanus toxin (TT)).

The affinities of the LGR5 and EGFR Fabs of a candidate EGFR/LGR5 bispecific antibody for their targets can be measured by surface plasmon resonance (SPR) technology using a BIAcore T100. Briefly, an anti-human IgG mouse monoclonal antibody (Becton and Dickinson, cat. Nr. 555784) is coupled to the surface of a CM5 sensor chip using free amine chemistry (NHS/EDC). Then the bispecific antibody is captured onto the sensor surface. Subsequently, the recombinant purified antigens human EGFR (Sino Biological Inc, cat. Nr. 11896-H07H) and human LGR5 protein are run over the sensor surface in a concentration range to measure on- and off-rates. After each cycle, the sensor surface is regenerated by a pulse of HCl and the bispecific antibody is captured again. From the obtained sensorgrams, on- and off-rates and affinity values for binding to human LGR5 and EGFR are determined using the BIAevaluation software, as previously described for CD3 in US 2016/0368988.

### ADCC

An antibody as disclosed herein is typically a bispecific full length antibody, in certain aspects of the human IgG subclass. In certain aspects, said antibody is of the human IgG1 subclass. Such antibodies have good ADCC properties which can, if desired, be enhanced by techniques known in the art, to have favorable half-life upon in vivo administration to humans or CH3 engineering technology that can provide for modified heavy chains that preferentially form heterodimers over homodimers upon co-expression in clonal cells.

ADCC activity of an antibody can be improved when the antibody itself has a low ADCC activity, by modifying the constant region of the antibody. Another way to improve ADCC activity of an antibody is by enzymatically interfering with the glycosylation pathway resulting in reduced fucose. Several in vitro methods exist for determining the efficacy of antibodies or effector cells in eliciting ADCC. Among these are chromium-51 [Cr51]release assays, europium [Eu]release assays, and sulfur-35 [S35]release assays. Usually, a labeled target cell line expressing a certain surface-exposed antigen is incubated with antibody specific for that antigen. After washing, effector cells expressing Fc receptor CD16 are co-incubated with the antibody-labeled target cells. Target cell lysis is subsequently measured by release of intracellular label by a scintillation counter or spectrophotometry.

A bispecific antibody as disclosed herein can be ADCC enhanced. In certain aspects, such a bispecific antibody is afucosylated. In certain aspects, a bispecific antibody comprises a reduced amount of fucosylation of the N-linked carbohydrate structure in the Fc region, when compared to the same antibody produced in a normal CHO cell. Low fucose levels are associated with increased CD16 (FcγRIIIa) binding on NK effector cells, resulting in increased ADCC activity. In certain aspects, and in addition to its direct antitumor activity, a bispecific antibody of the present disclosure can eliminate tumor cells following opsonization and subsequent natural killer (NK) cell mediated ADCC activity and complement-dependent cytotoxic (CDC) activity.

The antibody that comprises a variable domain that binds an extracellular part of EGFR and a variable domain that binds an extracellular part of LGR5 may further comprise one or more additional variable domains that can bind one or more further targets. In certain aspects, said further target is a protein, such as a membrane protein comprising an extracellular part. A membrane protein as used herein is a cell membrane protein, such as a protein that is in the outer membrane of a cell, the membrane that separates the cell from the outside world. The membrane protein has an extracellular part. A membrane protein is at least on a cell if it contains a transmembrane region that is in the cell membrane of the cell.

Antibodies with more than two variable domains are known in the art. For instance, it is possible to attach an additional variable domain. In certain aspects, an antibody with three or more variable domains is a multivalent multimer antibody as described in PCT/NL2019/050199 which is incorporated by reference herein.

In certain aspects, the antibody is a bispecific antibody comprising two variable domains, wherein one variable domain binds an extracellular part of EGFR and another variable domain binds an extracellular part of LGR5. In certain aspects, the variable domains are variable domains as described herein.

A functional part of an antibody as described herein comprises at least a variable domain that binds an extracellular part of EGFR and a variable domain that binds an extracellular part of LGR5 as described herein. It thus comprises the antigen binding parts of an antibody as described herein and typically contains the variable domains of the antibody. A variable domain of a functional part can be a single chain Fv-fragment or a so-called single domain antibody fragment. In certain aspects, the antibody parts or derivatives have at least two variable domains of an antibody or equivalents thereof. Non-limiting examples of such variable domains or equivalents thereof are F(ab)-fragments and Single chain Fv fragments. A functional part of a bispecific antibody comprises the antigen binding parts of the bispecific antibody, or a derivative and/or analogue of the binding parts. As mentioned herein above, the binding part of an antibody is encompassed in the variable domain.

### Pharmaceutical Compositions and Administration

Also provided is a pharmaceutical composition comprising an antibody or functional part, derivative and/or analogue thereof of the present disclosure and a fluoropyrimidine, a platinum-based chemotherapeutic agent, a BCL-2 inhibitor, or SN-38, as disclosed herein and a pharmaceutically acceptable carrier. In certain aspects, the disclosure provides a pharmaceutical composition comprising an EGFR/LGR5 bispecific antibody and a fluoropyrimidine, a platinum-based chemotherapeutic agent, a BCL-2 inhibitor, or SN-38. In certain aspects, the disclosure provides a combination, or a kit-of-parts, of a pharmaceutical composition comprising an EGFR/LGR5 bispecific antibody and a separate pharmaceutical composition comprising either a fluoropyrimidine, a platinum-based chemotherapeutic agent, a BCL-2 inhibitor or SN-38.

As used herein, the term “pharmaceutically acceptable” means approved by a government regulatory agency or listed in the U.S. Pharmacopeia or another generally recognized pharmacopeia for use in animals, particularly in humans, and includes any and all solvents, salts, dispersion media, coatings, antibacterial and antifungal agents, isotonic and absorption delaying agents, and the like that are physiologically compatible. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions. Liquid compositions for parenteral administration can be formulated for administration by injection or continuous infusion. Routes of administration by injection or infusion include intravesical, intratumoral, intravenous, intraperitoneal, intramuscular, intrathecal and subcutaneous.

Depending on the route of administration (e.g., intravenously, subcutaneously, intra-articularly and the like) the active compound may be coated in a material to protect the compound from the action of acids and other natural conditions that may inactivate the compound.

Said pharmaceutical composition comprising said antibody, functional part, derivative and/or analogue thereof that comprises a variable domain that binds an extracellular part of EGFR may be contained in a holder that is separate, meaning not physically linked, from a holder that contains said pharmaceutical composition comprising said fluoropyrimidine, platinum-based chemotherapeutic agent, BCL-2 inhibitor, or SN-38.

Pharmaceutical compositions suitable for administration to human patients are typically formulated for parenteral administration, e.g., in a liquid carrier, or suitable for reconstitution into liquid solution or suspension for intravenous administration. The compositions may be formulated in dosage unit form for ease of administration and uniformity of dosage. Also included are solid preparations which are intended for conversion, shortly before use, to liquid preparations for either oral or parenteral administration. Such liquid forms include solutions, suspensions and emulsions. The compositions and methods provided herein are particularly useful for the treatment of cancer in a patient, particularly head and neck cancer, gastric, esophageal, gastro-esophageal-junction cancer, non-small cell lung cancer or colorectal cancer.

Accordingly, the compositions and methods may be used in the treatment of various malignancies.

In certain aspects, the subject or patient is a mammal, in particular a human.

### Prior Treatment

In certain aspects, the subject has previously not been treated with an anti-cancer therapy. Worded differently, the cancer of said subject has previously not been treated with an anti-cancer therapy. In certain aspects, the subject is anti-cancer treatment naive subject. In certain aspects, treatment with the therapeutic agent of the present disclosure is first-line treatment. In certain aspects, the subject has previously not been treated with a neoadjuvant therapy. In certain aspects, the cancer is head and neck cancer, gastric, esophageal, gastro-esophageal-junction cancer, non-small cell lung cancer or colorectal cancer.

In certain aspects, the subject has previously been treated with an anti-cancer therapy. Worded differently, the cancer of said subject has previously been treated with an anti-cancer therapy. In certain aspects, the subject has received prior treatment with an anti-cancer therapy. Worded differently, the cancer of said subject has received prior treatment with an anti-cancer therapy.

In certain aspects, the subject has previously been treated with two or more anti-cancer therapies. Worded differently, the cancer of said subject has previously been treated with two or more anti-cancer therapies. In certain aspects, the subject has received prior treatment with two or more anti-cancer therapies. Worded differently, the cancer of said subject has received prior treatment with two or more anti-cancer therapies.

In certain aspects, the cancer is head and neck cancer, gastric, esophageal, gastro-esophageal-junction cancer, non-small cell lung cancer or colorectal cancer. In certain aspects, the subject has previously been treated with neoadjuvant therapy.

In certain aspects, the anti-cancer therapy is chemotherapy, radiation therapy, targeted therapy and/or immunotherapy.

The subject may have been previously treated with one or more lines of standard approved therapy or standard of care. Although surgery or radiation therapy may be preferred for most patients with early or localized disease, and may be considered for locally advanced disease, it may not be possible to apply to all patients, for instance due to the anatomical location of the cancer. In certain aspects, standard approved therapy or standard of care herein includes treatment by administration of a chemotherapeutic agent, such as one or more of a platinum-based chemotherapeutic agent (e.g. cisplatin, carboplatin, oxaliplatin), an antineoplastic compound (e.g. methotrexate), a fluoropyrimidine (e.g. fluorouracil, 5-FU, capecitabine), a taxane (e.g. docetaxel or paclitaxel) a nucleoside analogue (e.g. gemcitabine), a BCL-2 inhibitor or SN-38, or any combination thereof. In certain aspects, chemotherapy is fluorouracil, TAS-102, oxaliplatin, venetoclax, SN-38, FOLFOX or FOLFIRI.

Targeted therapy as disclosed herein, comprises treatment with an agent that blocks the action of a certain enzyme, protein or other molecule involved in the growth or spread of cancer cells. Targeted therapies include, but are not limited to, angiogenesis inhibitors (e.g. bevacizumab), or a monoclonal antibody (e.g. trastuzumab, cetuximab, afatinib). In certain aspects, the targeted therapy is cetuximab.

In certain aspects immunotherapy comprises treatment with an immune checkpoint inhibitor. In certain aspects, the immune checkpoint inhibitor of the present disclosure targets an immune checkpoint protein selected from PD-L1, PD-1, CTLA-4, B7-1 or B7-2. In certain aspects, the immune checkpoint inhibitor comprises durvalumab, pembrolizumab, ipilimumab, nivolumab, atezolizumab, retifanlimab cemiplimab or other anti-PD1, anti-PD-L1 antibodies approved or in development. In certain aspects, the immune checkpoint inhibitor comprises durvalumab or pembrolizumab.

In certain aspects, the subject to be treated has progressed after treatment with fluorouracil, oxaliplatin, FOLFOX, FOLFIRI, targeted therapy, immunotherapy or a combination thereof. In certain aspects, said treatment is for colorectal cancer.

In certain aspects, the subject or cancer to be treated has been previously treated with first line standard of care as appropriate for CRC or mCRC. In certain aspects, said subject or said cancer to be treated has been previously treated and is administered second line treatment using an antibody or functional part, derivative and/or analogue thereof that comprises a variable domain that binds an extracellular part of EGFR and optionally a variable domain that binds an extracellular part of LGR5, in combination with either a fluoropyrimidine, a platinum-based chemotherapeutic agent, a BCL-2 inhibitor, or SN-38.

In certain aspects, the subject or cancer to be treated has been previously treated and is administered third line treatment or later treatment, using an antibody or functional part, derivative and/or analogue thereof that comprises a variable domain that binds an extracellular part of EGFR and optionally a variable domain that binds an extracellular part of LGR5, in combination with either a fluoropyrimidine, a platinum-based chemotherapeutic agent, a BCL-2 inhibitor, or SN-38. In certain aspects, said prior treatment comprises standard of care treatment appropriate for CRC or mCRC. In certain aspects, said standard of care comprises or is chemotherapy appropriate for CRC or mCRC. In certain aspects, said prior treatment comprises or is fluoropyrimidine-oxaliplatin-based chemotherapy with or without bevacizumab or fluoropyrimidine-irinotecan-based chemotherapy with or without bevacizumab appropriate for CRC or mCRC.

In certain aspects, said subject or cancer has been previously treated with a fluoropyrimidine-oxaliplatin-based chemotherapy with or without bevacizumab and is administered an antibody or functional part, derivative and/or analogue thereof that comprises a variable domain that binds an extracellular part of EGFR and optionally a variable domain that binds an extracellular part of LGR5, and is administered FOLFIRI. In certain aspects, said subject or cancer has been previously treated with only said oxaliplatin-based chemotherapy. Said administration with said antibody or functional part, derivative and/or analogue thereof that comprises a variable domain that binds an extracellular part of EGFR and optionally a variable domain that binds an extracellular part of LGR5, and FOLFIRI is thus provided as second line therapy.

In certain aspects, said subject or cancer has been previously treated with a fluoropyrimidine-irinotecan-based chemotherapy with or without bevacizumab and is administered an antibody or functional part, derivative and/or analogue thereof that comprises a variable domain that binds an extracellular part of EGFR and optionally a variable domain that binds an extracellular part of LGR5, and is administered FOLFOX.

In certain aspects, said subject or cancer has been previously treated with only said irinotecan-based chemotherapy. Said administration with said antibody or functional part, derivative and/or analogue thereof that comprises a variable domain that binds an extracellular part of EGFR and optionally a variable domain that binds an extracellular part of LGR5, and FOLFIRI is thus provided as second line therapy.

In certain aspects, the subject to be treated has progressed after treatment with fluorouracil, oxaliplatin, FOLFOX, FOLFIRI, targeted therapy, immunotherapy or a combination thereof. In certain aspects, said treatment is for gastric cancer.

In certain aspects, the subject to be treated has progressed after treatment with a fluoropyrimidine, oxaliplatin, FOLFOX, FOLFIRI, targeted therapy, immunotherapy or a combination thereof. In certain aspects, said treatment is for esophageal, gastro-esophageal-junction cancer.

In certain aspects, the subject to be treated has progressed after treatment with radiation therapy, fluoropyrimidine, platinum-based therapy, targeted therapy, immunotherapy or a combination thereof. In certain aspects said treatment is for head and neck cancer.

In certain aspects, the subject to be treated has progressed after treatment with a platinum-based therapy, targeted therapy, immunotherapy or a combination thereof. In certain aspects, said treatment is for non-small cell lung cancer.

### Administration

In its Biclonics® antibody program, Merus has developed multispecific antibodies that target EGFR and LGR5 (Leucine-rich repeat containing G protein-coupled receptor). The efficacy of such multispecific antibodies has been assessed in vitro and in vivo using patient-derived CRC organoids and mice PDX models, respectively (see, e.g., WO2017/069628; which is incorporated by reference herein). Multispecific antibodies that target EGFR and LGR5 were shown to inhibit tumor growth. The potency of such inhibitory antibodies was shown to be correlated with the levels of LGR5 RNA expression by cells from the cancer. In certain aspects, said multispecific antibodies that target EGFR and LGR5 are as described in WO2017/069628.

The disclosure further provides methods of treating such cancer in a subject, the method comprising administering the subject in need thereof with the antibody or functional part, derivative and/or analogue thereof and a fluoropyrimidine, a platinum-based chemotherapeutic agent, a BCL-2 inhibitor, or SN-38 to the subject. In certain aspects, said method comprises providing the subject with a dose of 1500 mg of the antibody or functional part, derivative and/or analogue thereof. In certain aspects, said method comprises providing the subject with a flat dose of 1500 mg of the antibody or functional part, derivative and/or analogue thereof. In certain aspects, administrations of the therapeutic agent or the combination of agents to the subject may be done weekly, biweekly or monthly. In certain aspects, the therapeutic agent or the combination of agents is administered once every 2 weeks. In certain aspects, the therapeutic agent is petosemtamab and is administered once every 2 weeks at 1500 mg.

In certain aspects, administration of the antibody or functional part, derivative and/or analogue thereof, is at a dosage of between 5 and 2000 mg. In certain aspects, administration of the antibody or functional part, derivative and/or analogue thereof, is at a dosage of 5, 20, 50, 90, 150, 225, 335, 500, 750, 1100, 1500 mg or 2000 mg. In certain aspects, administration of the antibody or functional part, derivative and/or analogue thereof, is at a dosage of 750, 1100, 1500 mg or 2000 mg. In certain aspects, administration of the antibody or functional part, derivative and/or analogue thereof, is at a dosage of 750 mg. In certain aspects, administration of the antibody or functional part, derivative and/or analogue thereof, is at a dosage of 1100 mg. In certain aspects, administration of the antibody or functional part, derivative and/or analogue thereof, is at a dosage of 1500 mg. In certain aspects, administration of the antibody or functional part, derivative and/or analogue thereof, is at a dosage of 2000 mg. As is understood by the skilled person, the dosage can be administered over time. In certain aspects, the dosage is administered by IV, for example with a 1-6 hour infusion, or a 2-4 hour infusion. In certain aspects, said dosage is administered once every 2 weeks. In certain aspects, said dosage is a flat dosage which is suitable for use in adults and/or in subjects weighing at least 35 kg.

In certain aspects, administration of petosemtamab is at a dosage of between 5 and 2000 mg. In certain aspects, administration of petosemtamab is at a dosage of 5, 20, 50, 90, 150, 225, 335, 500, 750, 1100, 1500 mg or 2000 mg. In certain aspects, administration of petosemtamab is at a dosage of 750 mg. In certain aspects, administration of petosemtamab is at a dosage of 1100 mg. In certain aspects, administration of petosemtamab is at a dosage of 1500 mg. In certain aspects, administration of petosemtamab is at a dosage of 2000 mg. As is understood by the skilled person, the dosage can be administered over time. In certain aspects, the dosage is administered by IV, for example with a 1-6 hour infusion, or a 2-4 hour infusion. In certain aspects, said dosage is administered once every 2 weeks. In certain aspects, said dosage is a flat dosage which is suitable for use in adults and/or in subjects weighing at least 35 kg.

In certain aspects, administration of petosemtamab is at a weekly dosage of 750 mg. In certain aspects, administration of petosemtamab is at a biweekly dosage of 750 mg. In certain aspects, administration of petosemtamab is at a triweekly dosage of 750 mg.

In certain aspects, administration of petosemtamab is at a weekly dosage of 1100 mg. In certain aspects, administration of petosemtamab is at a biweekly dosage of 1100. In certain aspects, administration of petosemtamab is at a triweekly dosage of 1100 mg.

In certain aspects, administration of petosemtamab is at a weekly dosage of 1500 mg. In certain aspects, administration of petosemtamab is at a biweekly dosage of 1500 mg. In certain aspects, administration of petosemtamab is at a triweekly dosage of 1500 mg.

In certain aspects, said antibody or functional part, derivative and/or analogue thereof comprises petosemtamab and is administrated in an amount which achieves human receptor target engagement for both EGFR and LGR5 of at least 90%, at least 95%, at least 99% across relevant body weights for a statistically significant number of subjects. Said amount of 90% may be achieved using a flat dose of about 1000 mg Q2W. Said amount of 95% may be achieved using a flat dose of about 1100 to about 1200 mg Q2W.

In certain aspects, a premedication regimen is used. Such a regimen may be useful to reduce the likelihood or severity of an infusion-related reaction. Generally, a steroid such as dexamethasone and/or an antihistamine such as dexchlorpheniramine, diphenhydramine, or chlorpheniramine is administered (e.g., orally, intravenously) prior to treatment with a therapeutic agent mentioned herein. In certain aspects, treatment of the present disclosure comprises premedication with paracetamol/acetaminophen, antihistamines, or corticosteroids. In certain aspects, said premedication is administered in the event of infusion-related reactions, hypersensitivity and/or allergic reactions, according to standard local clinical practice. In certain aspects, a dose of 1500 mg petosemtamab is premedicated with an antihistamine, pain reducing medication, fever reducing medication and/or anti-inflammatory medication. In certain aspects, a dose of 750 mg petosemtamab is premedicated with an antihistamine, pain reducing medication, fever reducing medication and/or anti-inflammatory medication.

As used herein, combined treatment, administration or co-administration includes simultaneous treatment or administration of an antibody or functional part, derivative and/or analogue thereof of the present disclosure and a fluoropyrimidine, a platinum-based chemotherapeutic agent, a BCL-2 inhibitor, or SN-38, in the same or different dosage form, separate administration or sequential administration. Accordingly, in certain aspects, said antibody or functional part, derivative and/or analogue thereof may be used in a method for treating cancer in a subject, wherein said antibody or functional part, derivative and/or analogue thereof is administered simultaneously, separately or sequentially with a fluoropyrimidine, a platinum-based chemotherapeutic agent, a BCL-2 inhibitor, or SN-38. In other aspects, said antibody or functional part, derivative and/or analogue thereof may be used in the treatment of a cancer in a subject, wherein said antibody or functional part, derivative and/or analogue thereof may be administered simultaneously, separately or sequentially with a fluoropyrimidine, a platinum-based chemotherapeutic agent, a BCL-2 inhibitor, or SN-38.

In some aspects, an antibody or functional part, derivative and/or analogue thereof may be for use in the manufacture of a medicament for the treatment of cancer in a subject, wherein said antibody or functional part, derivative and/or analogue thereof is administered simultaneously, separately or sequentially with a fluoropyrimidine, a platinum-based chemotherapeutic agent, a BCL-2 inhibitor, or SN-38. In some aspects, said antibody or functional part, derivative and/or analogue thereof may be for use in the manufacture of a medicament for treating a cancer in a subject, wherein said antibody or functional part, derivative and/or analogue thereof may be administered simultaneously, separately or sequentially with a fluoropyrimidine, a platinum-based chemotherapeutic agent, a BCL-2 inhibitor, or SN-38.

In some aspects, said antibody or functional part, derivative and/or analogue thereof and a fluoropyrimidine, a platinum-based chemotherapeutic agent, a BCL-2 inhibitor, or SN-38, may be for use in the manufacture of one or more medicaments, for the treatment of a cancer in a subject, wherein said antibody or functional part, derivative and/or analogue thereof is administered simultaneously, separately or sequentially with a fluoropyrimidine, a platinum-based chemotherapeutic agent, a BCL-2 inhibitor, or SN-38.

A product comprising said antibody or functional part, derivative and/or analogue thereof and a fluoropyrimidine, a platinum-based chemotherapeutic agent, a BCL-2 inhibitor, or SN-38, may be a combined preparation for simultaneous, separate or sequential use in treating a cancer in a subject.

Said antibody or functional part, derivative and/or analogue thereof and the fluoropyrimidine, a platinum-based chemotherapeutic agent, a BCL-2 inhibitor, or SN-38 can also be administered according to any suitable schedule. In certain aspects, said antibody or functional part, derivative and/or analogue thereof and the fluoropyrimidine, platinum-based chemotherapeutic agent, BCL-2 inhibitor, or SN-38, can be simultaneously administered in a single formulation. In certain aspects, said antibody or functional part, derivative and/or analogue thereof and the fluoropyrimidine, platinum-based chemotherapeutic agent, BCL-2 inhibitor, or SN-38, can be formulated for separate administration, wherein they are administered concurrently or sequentially.

For example, in certain aspects, said antibody or functional part, derivative and/or analogue thereof can be administered first followed by the administration of the fluoropyrimidine, the platinum-based chemotherapeutic agent, a BCL-2 inhibitor, or SN-38, or vice versa. Dosage regimens in the above methods of treatment and uses are adjusted to provide the optimum desired response (e.g., a therapeutic response).

Said antibody or functional part, derivative and/or analogue thereof and the fluoropyrimidine, the platinum-based chemotherapeutic agent, the BCL-2 inhibitor, or SN-38 disclosed herein, can be administered according to a suitable dosage, and suitable route (e.g., intravenous, intraperitoneal, intramuscular, intrathecal or subcutaneous). For example, a single bolus may be administered, several divided doses may be administered over time or the dose may be proportionally reduced or increased as indicated by the exigencies of the therapeutic situation.

In certain aspects, the disclosure provides a method of treating cancer in a subject, wherein the method comprises administering an effective amount of said antibody or functional part, derivative and/or analogue thereof, and an effective amount of the fluoropyrimidine, platinum-based chemotherapeutic agent, BCL-2 inhibitor, or SN-38, to the subject.

In certain aspects, said antibody or functional part, derivative and/or analogue thereof is administered prior to administration of the fluoropyrimidine, the platinum-based chemotherapeutic agent, the BCL-2 inhibitor, or SN-38, e.g., said antibody or functional part, derivative and/or analogue thereof is administered into the patient first, followed later by an administration of the fluoropyrimidine, the platinum-based chemotherapeutic agent, the BCL-2 inhibitor, or SN-38. In one aspect, the fluoropyrimidine, the platinum-based chemotherapeutic agent, the BCL-2 inhibitor, or SN-38 is administered prior to administration of said antibody or functional part, derivative and/or analogue thereof e.g., the fluoropyrimidine, the platinum-based chemotherapeutic agent, the BCL-2 inhibitor, or SN-38 is administered into the patient first, followed later by administration of said antibody or functional part, derivative and/or analogue thereof (e.g., one or more minutes, hours or days after). Such concurrent or sequential administration results in both said antibody or functional part, derivative and/or analogue thereof and the fluoropyrimidine, the platinum-based chemotherapeutic agent, the BCL-2 inhibitor, or SN-38 being simultaneously present in treated patients. Concurrent presence of both said antibody or functional part, derivative and/or analogue thereof and the fluoropyrimidine, the platinum-based chemotherapeutic agent, the BCL-2 inhibitor, or SN-38 will support cancer treatment induced by said antibody or functional part, derivative and/or analogue thereof and inhibition of EGFR/LGR5 signaling mediated by said antibody or functional part, derivative and/or analogue thereof.

In certain aspects, a subject is administered a single dose of said antibody or functional part, derivative and/or analogue thereof and a single dose of the fluoropyrimidine, the platinum-based chemotherapeutic agent, the BCL-2 inhibitor, or SN-38 as disclosed herein. In certain aspects, said antibody or functional part, derivative and/or analogue thereof and the fluoropyrimidine, the platinum-based chemotherapeutic agent, the BCL-2 inhibitor, or SN-38 will be administered repeatedly, over a course of treatment. For example, in certain aspects, multiple (e.g., 2, 3, 4, 5, 6, 7, 8, 9, 10 or more) doses of a fluoropyrimidine, a platinum-based chemotherapeutic agent, a BCL-2 inhibitor, or SN-38 and multiple (e.g., 2, 3, 4, 5, 6, 7, 8, 9, 10 or more) doses of said antibody or functional part, derivative and/or analogue thereof are administered to a subject in need of treatment.

In certain aspects, administrations of said antibody or functional part, derivative and/or analogue thereof and the fluoropyrimidine, the platinum-based chemotherapeutic agent, the BCL-2 inhibitor, or SN-38 may be done weekly, biweekly or monthly, in which regimen, they may be administered on the same day (e.g., simultaneously), or one after the other (e.g., one or more minutes, hours or days before or after one another). When administered separately, said antibody or functional part, derivative and/or analogue thereof and the fluoropyrimidine, the platinum-based chemotherapeutic agent, the BCL-2 inhibitor, or SN-38 may be, but are not necessarily administered according to the same administration (i.e., dosing) protocol. For example, one cycle of treatment may comprise administering said antibody or functional part, derivative and/or analogue thereof one or multiple times, while a therapeutically effective dose of the fluoropyrimidine, the platinum-based chemotherapeutic agent, the BCL-2 inhibitor, or SN-38 may be administered either more or less frequently than said antibody or functional part, derivative and/or analogue thereof. In certain aspects, administration of each dose of the fluoropyrimidine, the platinum-based chemotherapeutic agent, the BCL-2 inhibitor, or SN-38 and said antibody or functional part, derivative and/or analogue thereof may be on the same day, or alternatively, the fluoropyrimidine, the platinum-based chemotherapeutic agent, the BCL-2 inhibitor, or SN-38 may be administered one or more days before or after said antibody or functional part, derivative and/or analogue thereof.

In certain aspects, the dose of said antibody or functional part, derivative and/or analogue thereof and/or the fluoropyrimidine, the platinum-based chemotherapeutic agent, the BCL-2 inhibitor, or SN-38 is varied over time. For example, said antibody or functional part, derivative and/or analogue thereof and/or the fluoropyrimidine, the platinum-based chemotherapeutic agent, the BCL-2 inhibitor, or SN-38 may be initially administered at a high dose and may be lowered over time. In another aspect, said antibody or functional part, derivative and/or analogue thereof and/or the fluoropyrimidine, the platinum-based chemotherapeutic agent, the BCL-2 inhibitor, or SN-38 is initially administered at a low dose and increased over time.

In certain aspects, the amount of said antibody or functional part, derivative and/or analogue thereof and/or the fluoropyrimidine, the platinum-based chemotherapeutic agent, the BCL-2 inhibitor, or SN-38 is constant for each dose. In another aspect, the amount of said antibody or functional part, derivative and/or analogue thereof and/or the fluoropyrimidine, the platinum-based chemotherapeutic agent, the BCL-2 inhibitor, or SN-38 varies with each dose. For example, the maintenance (or follow-on) dose of each can be higher or the same as the loading dose which is first administered. In another aspect, the maintenance dose of each can be lower or the same as the loading dose. A clinician may utilize preferred dosages as warranted by the condition of the patient being treated. The dose may depend upon a number of factors, including stage of disease, etc. The specific dose that should be administered based upon the presence of one or more of such factors is within the skill of the artisan. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In certain aspects, said antibody or functional part, derivative and/or analogue thereof is administered at a dose of 0.1, 0.3, 1, 2, 3, 4, 5, 6, 7, 8, 9, or 10 mg/kg body weight. Alternatively, said antibody or functional part, derivative and/or analogue thereof is administered at a dose of 0.5, 1, 2, 3, 4, 5, 6, 7, 8, 9, or 10 mg/kg body weight. In certain aspects, said antibody or functional part, derivative and/or analogue thereof is provided to a subject using a flat dosage of 1500 mg. A flat dosage offers several advantages over body-surface or weight dosing as it reduces preparation time and reduces potential dose calculation mistakes. In certain aspects, said antibody or functional part, derivative and/or analogue thereof is provided at a dosage of at least 500 mg. In certain aspects, said dosage is between 1100 to 2000 mg. In certain aspects, said dosage is between 1100 to 1800 mg. As is understood by the skilled person, the dosage can be administered over time. For example, the dosage may be administered by IV, for example with a 1-6 hour infusion, or a 2-4 hour infusion. In certain aspects, said antibody or functional part, derivative and/or analogue thereof is administered once every 2 weeks. In certain aspects, the antibody is petosemtamab and is administered once every 2 weeks at 1500 mg flat dose. In particular, the flat dosages disclosed herein are suitable for use in adults and/or in subjects weighing at least 35 kg. In certain aspects, the subject is afflicted with head and neck cancer, gastric, esophageal, gastro-esophageal-junction cancer, non-small cell lung cancer or colorectal cancer.

In certain aspects, a premedication regimen may be used. Such a regimen may be useful to reduce the likelihood or severity of an infusion-related reaction. Generally, a steroid such as dexamethasone and/or an antihistamine such as dexchlorpheniramine, diphenhydramine, or chlorpheniramine is administered (e.g., orally, intravenously) prior to antibody treatment.

The treatment method described herein is typically continued for as long as the clinician overseeing the patient's care deems the treatment method to be effective, i.e., that the patient is responding to treatment. Non-limiting parameters that indicate the treatment method is effective may include one or more of the following: decrease in tumor cells; inhibition of tumor cell proliferation; tumor cell elimination; progression-free survival; appropriate response by a suitable tumor marker (if applicable).

With regard to the frequency of administering said antibody or functional part, derivative and/or analogue thereof and/or the fluoropyrimidine, the platinum-based chemotherapeutic agent, the BCL-2 inhibitor, or SN-38, one of ordinary skill in the art will be able to determine an appropriate frequency. For example, a clinician can decide to administer said antibody or functional part, derivative and/or analogue thereof and/or the fluoropyrimidine, the platinum-based chemotherapeutic agent, the BCL-2 inhibitor, or SN-38 relatively infrequently (e.g., once every two weeks) and progressively shorten the period between doses as tolerated by the patient. Exemplary lengths of time associated with the course of therapy in accordance with the claimed method include: about one week; about two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years; about five years; perpetual (e.g., ongoing maintenance therapy). The foregoing duration may be associated with one or multiple rounds/cycles of treatment.

The efficacy of the treatment methods provided herein can be assessed using any suitable means. In certain aspects, the clinical efficacy of the treatment is analyzed using cancer cell number reduction as an objective response criterion. Patients, e.g., humans, treated according to the methods disclosed herein, experience improvement in at least one sign of cancer. In certain aspects, one or more of the following can occur: the number of cancer cells can be reduced; cancer recurrence is prevented or delayed; one or more of the symptoms associated with cancer can be relieved to some extent. In addition, in vitro assays determine the T cell mediated target cell lysis. In certain aspects, tumor assessment is based on CT-scan and/or MRI scans, see, e.g., the RECIST 1.1 guidelines (Response Evaluation Criteria in Solid Tumours) (Eisenhauer et al., 2009 Eur J Cancer 45:228-247). Such assessments generally take place every 4-8 weeks after treatment.

In certain aspects, the tumor cells are no longer detectable following treatment as described herein. In certain aspects, a subject is in partial or full remission. In certain aspects, a subject has an increased overall survival, median survival rate, and/or progression free survival.

The combination of the present disclosure (i.e., said antibody or functional part, derivative and/or analogue thereof in combination with fluoropyrimidine, platinum-based chemotherapeutic agent, BCL-2 inhibitor, or SN-38) may also be used in conjunction with other well-known therapies (e.g., chemotherapy or radiation therapy) that are selected for their particular usefulness against the cancer that is being treated.

Methods for the safe and effective administration of chemotherapeutic agents are known to those skilled in the art. In addition, their administration is described in the standard literature. For example, the administration of many of the chemotherapeutic agents is described in the Physicians' Desk Reference (PDR), e.g., 1996 edition (Medical Economics Company, Montvale, N.J. 07645-1742, USA); the disclosure of which is incorporated herein by reference thereto.

It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

In certain aspects, at the start of treatment, at least one, more than one or all of the following inclusion factors IF1-IF16 are applicable to subjects for treatment. In certain aspects, the subject comprises or complies with all of IF1-IF16:
IF 1. Having an age of at least 18.
IF 2. Having histologically or cytologically confirmed solid tumors with evidence of metastatic or locally advanced disease not amenable to standard therapy with curative intent, or locally advanced unresectable or metastatic disease.
IF 3. Having been previously diagnosed with histologically or cytologically confirmed unresectable or metastatic adenocarcinoma of the colon or rectum. Subjects are RAS/RAF wildtype as determined using NGS on tumor tissue (primary or metastatic).
IF4. Subjects are naive to prior anti-EGFR therapy.
IF5. Disease progression must have occurred during or within 6 months of prior first line chemotherapy as received and confirmed by suitable radiographic testing.
IF6.1. Subjects for treatment with a combination of petosemtamab and FOLFIRI should have only received a single prior chemotherapy regimen for the metastatic setting, which typically is first line fluoropyrimidine-oxaliplatin-based chemotherapy f bevacizumab.
IF6.2. Subjects for treatment with a combination of petosemtamab and FOLFOX should have received only a single prior chemotherapy regimen for the metastatic setting, which typically is first line fluoropyrimidine-irinotecan-based chemotherapy with or without bevacizumab.
IF 7. Having a new baseline tumor sample (e.g. a formalin-fixed paraffin-embedded block, [FFPE]) from a metastatic or primary site. If the subject has such an available tumor sample from sample collection with sufficient material (at least 20 slides with >20% tumor content) and has not received further anticancer treatment since said sample collection, a new tumor biopsy at baseline is not necessary. Archival FFPE slides are not acceptable.
IF 8. Being amenable for a biopsy.
IF 9. Having a measurable disease as defined by RECIST version 1.1 by radiologic methods.
IF 10. Having an Eastern Cooperative Oncology Group (ECOG) performance status of 0 or 1.
IF 11. Life expectancy ≥12 weeks, typically assessed as per investigator.
IF 12. Left ventricular ejection fraction (LVEF) at least 50% by echocardiogram (ECHO) or multigated acquisition scan (MUGA).
IF 13. Adequate organ function:
   IF 13.1 Having an Absolute Neutrophil Count (ANC) of at least 1.5×10⁹/L.
   IF 13.2 Having a hemoglobin level of at least 9 g/dL.
   IF 13.3 Having a platelets level of at least 100×10⁹/L.
   IF 13.4 Having a corrected total serum calcium within normal ranges.
   IF 13.5 Having serum magnesium, sodium, corrected total calcium, phosphate, and potassium within normal ranges (or corrected with supplements or appropriate treatment).
   IF 13.6 Having an Alanine aminotransferase (ALT), Aspartate aminotransferase (AST) of equal to or less than 2.5 times the upper limit of normal (ULN) and total bilirubin of equal to or less than 1.5 times ULN, with the proviso that in case subjects have Gilbert's syndrome, total bilirubin is less than or equal to 3.0 times ULN or direct bilirubin is less than or equal to 1.5 times ULN; with the proviso that in case of liver involvement, ALT/AST is equal to or less than 5 times ULN and total bilirubin is equal to or less than 2 times ULN.
   IF 13.7 Having a serum creatinine level of less than or equal to 1.5 x ULN or having a creatinine clearance of at least 60 mL/min calculated according to the Cockroft and Gault formula or Modification of Diet in Renal Disease (MDRD) formula for patients of more than 65 years.
   IF 13.8 Having a serum albumin level of at least 3 g/dL.
   IF 13.9 Having am International Normalized Ratio (INR) or Prothrombin Time (PT) level of less than or equal to 1.5 x ULN, unless patient receives anticoagulant therapy and in therapeutic range of intended used anticoagulant.
   IF 13.10 Having an Activated Partial Thromboplastin Time (APTT) or PTT less than or equal to 1.5 x ULN, unless patient is receiving anticoagulant therapy and is in therapeutic range of intended used anticoagulant.
IF 14. Subject is willing to undergo testing for human immunodeficiency virus (HIV) if not tested within the past 6 months. Known HIV-positive subjects are eligible provided the cluster of differentiation 4 (CD4+) count is >300/μL, viral load is undetectable, and the patient is currently receiving highly active antiretroviral therapy (HAART).
IF 15. Being hepatitis B positive (HBsAg) but receive antiviral treatment with lamivudine, tenofovir, entecavir, or other antiviral agents, starting at least seven days start of treatment according to the present disclosure.
IF 16. Having an antecedent of hepatitis B (being anti-HBc positive, HBsAg and hepatitis B virus [HBV]-DNA negative).
IF 17. Having a positive test for hepatitis C virus (HCV) ribonucleic acid (RNA), with the proviso that said HCV infection resolved spontaneously (i.e. having positive HCV antibodies without detectable HCV-RNA) or that a subject who achieved a sustained response after antiviral treatment shows absence of detectable HCV RNA at least six months with the use of IFN-free regimens or at least 12 months with the use of IFN-based regimens, after cessation of antiviral treatment.

In certain aspects, all values for organ function measurements according to IF13 have an upper limit observed with healthy subjects.

In certain aspects, the subject for treatment complies with any one or more factors selected from the group consisting of IF1-IF16. In certain aspects, the subject for treatment complies with factors IF2, IF3, IF4, IF5, IF6, IF9, IF10, IF12, IF13, IF14, IF15, IF16 and IF17. In certain aspects, the subject for treatment complies with factors IF2, IF3, IF4, IF5 and IF6. In certain aspects, the subject for treatment complies with factor IF3 and IF6. In certain aspects, at the start of treatment, at least one, more than one or all of the following exclusion factors EF1-EF18 are applicable to subjects for treatment:
EF 1. Having central nervous system metastases that are untreated or symptomatic, or require radiation, surgery, or continued steroid therapy to control symptoms within 14 days from start of treatment according to the present disclosure.
EF 2. Having leptomeningeal involvement.
EF 3. Participation in a further clinical trial or treatment with any investigational drug within four weeks prior to start of treatment according to the present disclosure.
EF 4. Having any systemic anticancer therapy within four weeks or five half-lives, whichever is longer, of the first dose according to the present disclosure. For cytotoxic agents that have major delayed toxicity (e.g. mitomycin C, nitrosoureas), or anticancer immunotherapies, a washout period of six weeks prior to prior to start of treatment according to the present disclosure is required.
EF 5. Having a requirement for immunosuppressive medication (eg, methotrexate, cyclophosphamide).
EF 6. Having major surgery or radiotherapy within three weeks of start of treatment according to the present disclosure. Patients who received prior radiotherapy to at least 25% or more of bone marrow are excluded, irrespective of when it was received.
EF 7. Having persistent Grade >1 clinically significant toxicities related to prior antineoplastic therapies (except for alopecia); with the proviso that stable sensory neuropathy of Grade 2 (or less) National Cancer Institute-Common Terminology Criteria for Adverse Events (NCI-CTCAE) v4.03 or v5.0 is allowed or as current at the time of administration.
EF 8. Having a history any of the excipients of petosemtamab, human proteins, or any non-IMP treatment required for this study.
EF 9. Exhibiting uncontrolled hypertension (systolic BP>150 mmHg and/or diastolic BP>100 mmHg) with appropriate treatment, or unstable angina.
EF 10. Having a history of congestive heart failure of Class II-IV New York Heart Association (NYHA) criteria, or serious cardiac arrhythmia requiring treatment (except atrial fibrillation, paroxysmal supraventricular tachycardia).
EF 11. Having had a myocardial infarction within six months from start of treatment according to the present disclosure.
EF 12. Having a history of prior malignancies, with the exception of excised cervical intraepithelial neoplasia or nonmelanoma skin cancer, or curatively treated cancer deemed at low risk for recurrence with no evidence of disease for at least three years prior to start of treatment according to the present disclosure.
EF 13. Having dyspnea at rest of any origin, or other diseases requiring continuous oxygen therapy.
EF 14. Having a history of interstitial lung disease (ILD) (eg, pneumonitis or pulmonary fibrosis), or evidence of ILD on baseline chest computerized tomography (CT) scan.
EF 15. Having a serious illness or medical conditions including, but not limited to, uncontrolled active infection, clinically significant pulmonary, metabolic or psychiatric disorders at start of treatment according to the present disclosure.
EF 16. Subjects having an active hepatitis B surface antigen infection (HBsAg positive) without receiving antiviral treatment.
EF 17. Having a positive test for hepatitis C virus (HCV)
EF 18. Subjects having cirrhotic status of Child-Pugh class B or C; subjects having fibrolamellar HCC, subjects having sarcomatoid HCC, or subjects having mixed cholangiocarcinoma and HCC.
EF 18. Pregnant subjects or being a breastfeeding subject; subjects of childbearing potential must use highly effective contraception methods prior to start of treatment according to the present disclosure, for the duration of said treatment, and for six months after the last dose of petosemtamab administration.

In certain aspects, the subject for treatment complies with any one or more factors selected from the group consisting of EF1-EF18. In certain aspects, the subject for treatment complies with all of the factors EF1-EF18. In certain aspects, the subject for treatment complies with factors IF13, IF16.

ECOG Performance Status Scoring Grade Definition is as followed in the art, meaning: 0 Fully active, able to carry on all pre-disease performance without restriction. 1 Restricted in physically strenuous activity but ambulatory and able to carry out work of a light or sedentary nature, e.g., light housework, office work. 2 Ambulatory and capable of all self-care but unable to carry out any work activities. Up and about more than 50% of waking hours. 3 Capable of only limited self-care, confined to bed or chair more than 50% of waking hours. 4 Completely disabled. Cannot carry on any self-care. Totally confined to bed or chair. 5 Dead.

The Child-Pugh scoring, also referred to as the Child-Pugh classification, the Child-Turcotte-Pugh (CTP) calculator or the Child Criteria, herein is applied in accordance with standard clinical practice. The Child-Pugh score is determined by scoring five clinical measures of liver disease and the possibility of eventual liver failure. A score of 1, 2, or 3 is given to each measure, with 3 being the most severe. The five clinical measures are total bilirubin, serum albumin level, prothrombin time (or prolongation or INR as a time for blood to clot), ascites, and hepatic encephalopathy. Class A means 5 to 6 points, least severe liver disease and a one- to five-year survival rate of 95 percent. Class B: 7 to 9 points, moderately severe liver disease and a one- to five-year survival rate of 75 percent. Class C: to 15 points, most severe liver disease and a one- to five-year survival rate of 50 percent. Per clinical measure, the following points are given. Encephalopathy: None=1 p, Grade 1 and 2=2 p, Grade 3 and 4=3 pts. Ascites: none=1 pt, slight=2 pts, moderate=3 pts. Bilirubin: under 2 mg/ml=1 pt, 2 to 3 mg/ml=2 pts, over 3 mg/ml=3 pts. Albumin: greater than 3.5 mg/ml=1 pt, 2.8 to 3.5 mg/ml=2 pts, less than 2.8 mg/ml=3 pts. Prothrombin Time (PT, seconds prolonged): less than 4 sec=1 pt, 4 to 6 sec=2 pts, over 6 sec=3 pts. Alternatively, the International Normalized Ratio (INR) will be used as a substitute for PT, with INR under 1.7=1 pt, INR 1.7 to 2.2=2 pts, INR above 2.2=3 pts.

Herein, calculation of renal clearance is according to Cockcroft & Gault formula for subjects aged <65 years: Male=1.25×weight (kg)×(140-age)/serum creatinine (μmol/L). Female=1.04×weight (kg)×(140-age)/serum creatinine (μmol/L).

Herein, calculation of MDRD (Modification of Diet in Renal Disease) is according to the following formula: Patients aged >65 years. For a male subject older than 65 years of age, calculation is as follows: Male=186×(serum creatinine (μmol/L)×0,0113)−1,154×age−0,203. For dark skinned subjects, the outcome is multiplied by 1.21. For female subjects, the outcome is multiplied by 0.742.

### Kit of Parts and Combinations of Therapeutic Agents

In certain aspects, the disclosure provides a kit or a product which includes a pharmaceutical composition containing an EGFR/LGR5 bispecific antibody and a fluoropyrimidine, a platinum-based chemotherapeutic agent, a BCL-2 inhibitor, or SN-38, and a pharmaceutically-acceptable carrier, in a therapeutically effective amount adapted for use in the preceding methods. In certain aspects, the kit or product optionally also can include instructions, e.g., comprising administration schedules, to allow a practitioner (e.g., a physician, nurse, or patient) to administer the composition contained therein to a patient having cancer.

Therefore, in certain aspects, the disclosure provides a kit of parts comprising
an antibody or functional part, derivative and/or analogue thereof,
a fluoropyrimidine, a platinum-based chemotherapeutic agent, a BCL-2 inhibitor, or SN-38; and
instructions for use of EGFR/LGR5 bispecific antibody and instructions for use of the fluoropyrimidine, platinum-based chemotherapeutic agent, BCL-2 inhibitor, or SN-38.

In certain aspects, the instructions for use of petosemtamab comprise instructions for dosing at 1500 mg. In certain aspects, the instructions for use is for the use of said antibody or functional part, derivative and/or analogue thereof, the fluoropyrimidine, the platinum-based chemotherapeutic agent, the BCL-2 inhibitor, or SN-38, in the treatment of adenocarcinoma or a squamous cell carcinoma. In certain aspects, the instructions for use is for the treatment of head and neck cancer, gastric, esophageal, gastro-esophageal-junction cancer, non-small cell lung cancer or colorectal cancer. In certain aspects, the instructions for use is for the treatment of colorectal cancer.

In certain aspects, the kit or product includes multiple packages of the single-dose pharmaceutical compositions each containing an effective amount of an antibody or functional part, derivative and/or analogue thereof and a fluoropyrimidine, a platinum-based chemotherapeutic agent, a BCL-2 inhibitor, or SN-38, for a single administration in accordance with the methods provided above. Instruments or devices necessary for administering the pharmaceutical composition(s) also may be included in the kit or product. For instance, a kit or product may provide one or more pre-filled syringes containing a unit dosage of an antibody or functional part, derivative and/or analogue thereof and a fluoropyrimidine, a platinum-based chemotherapeutic agent, a BCL-2 inhibitor, or SN-38, in the same container, or in separate containers to be administered as separate and distinct compositions.

In certain aspects, one or both of an antibody or functional part, derivative and/or analogue thereof and a fluoropyrimidine, a platinum-based chemotherapeutic agent, a BCL-2 inhibitor, or SN-38 is provided in a solid form suitable for reconstitution and subsequent administration in accordance with the accompanying instructions.

In certain aspects, the disclosure provides a combination of antibody or functional part, derivative and/or analogue thereof and a fluoropyrimidine, a platinum-based chemotherapeutic agent, a BCL-2 inhibitor, or SN-38, for use in the treatment of cancer in a subject in need thereof.

In certain aspects, the disclosure provides a combination of:
an antibody or functional part, derivative and/or analogue thereof of the present disclosure,
instructions for use of said antibody or functional part, derivative and/or analogue thereof in the treatment of a cancer in a subject,
instructions for use in the treatment of a cancer in a subject of a fluoropyrimidine, a platinum-based chemotherapeutic agent, a BCL-2 inhibitor, or SN-38.

In certain aspects, the disclosure provides a combination of:
a fluoropyrimidine, a platinum-based chemotherapeutic agent, a BCL-2 inhibitor, or SN-38,
instructions for use of the fluoropyrimidine, the platinum-based chemotherapeutic agent, the BCL-2 inhibitor, or SN-38 in the treatment of cancer in a subject,
and instructions for use of said antibody or functional part, derivative and/or analogue thereof in the treatment of cancer in a subject.

In certain aspects, the instructions for use comprise the amount of said antibody or functional part, derivative and/or analogue thereof and/or the amount of fluoropyrimidine, platinum-based chemotherapeutic agent, BCL-2 inhibitor, or SN-38, to be used and/or the dosing interval and/or the cancer to be treated.

In yet further aspects, the composition or combination or kit or product includes one or more additional active agents.

The compounds and compositions disclosed herein are useful as therapy and in therapeutic treatments and may thus be useful as medicaments and used in a method of preparing a medicament.

In certain aspects, the present disclosure provides a pharmaceutical composition comprising an antibody or functional part, derivative and/or analogue thereof that comprises a variable domain that can bind an extracellular part of EGFR of the present disclosure and instructions for use thereof with a fluoropyrimidine, a platinum-based chemotherapeutic agent, a BCL-2 inhibitor, or SN-38, in the treatment of said cancer.

In certain aspects, the present disclosure provides a pharmaceutical composition for the treatment of a cancer, comprising an antibody or functional part, derivative and/or analogue thereof that comprises a variable domain that can bind an extracellular part of EGFR of the present disclosure and a pharmaceutical composition for the treatment of said cancer, comprising a fluoropyrimidine, a platinum-based chemotherapeutic agent, a BCL-2 inhibitor, or SN-38 of the present disclosure.

In certain aspects, the present disclosure provides a pharmaceutical composition for use in the treatment of cancer comprising an antibody or functional part, derivative and/or analogue thereof that comprises a variable domain that can bind an extracellular part of EGFR of the present disclosure, wherein the pharmaceutical composition is administered in combination with a fluoropyrimidine, a platinum-based chemotherapeutic agent, a BCL-2 inhibitor, or SN-38 of the present disclosure.

In certain aspects, the present disclosure relates to a pharmaceutical composition for the treatment of a cancer comprising an antibody or functional part, derivative and/or analogue thereof that comprises a variable domain that can bind an extracellular part of EGFR of the present disclosure, wherein the subject to be treated is administered a fluoropyrimidine, a platinum-based chemotherapeutic agent, a BCL-2 inhibitor, or SN-38 prior to, simultaneously with, or after administration of said bispecific antibody.

In certain aspects, the present disclosure relates to a pharmaceutical composition for the treatment of a cancer in a subject comprising a fluoropyrimidine, a platinum-based chemotherapeutic agent, a BCL-2 inhibitor, or SN-38, wherein said subject to be treated is administered an antibody or functional part, derivative and/or analogue thereof that comprises a variable domain that can bind an extracellular part of EGFR of the present disclosure prior to, simultaneously with, or after administration of said fluoropyrimidine, platinum-based chemotherapeutic agent, BCL-2 inhibitor, or SN-38.

The present disclosure thus relates to a combination of medicaments for the treatment of cancer in a subject which comprises administration to said subject of multiple, different medicaments for treating said cancer, which treatment comprises simultaneous, sequential or separate administration of said medicaments. In certain aspects, said medicament comprises an antibody or functional part, derivative and/or analogue thereof that comprises a variable domain that can bind an extracellular part of EGFR of the present disclosure, and said other, different medicament comprises a fluoropyrimidine, a platinum-based chemotherapeutic agent, a BCL-2 inhibitor, or SN-38.

In certain aspects, the antibody or functional part, derivative and/or analogue thereof that comprises a variable domain that can bind an extracellular part of EGFR of the present disclosure may be administered simultaneously, sequentially or separately with the fluoropyrimidine, the platinum-based chemotherapeutic agent, the BCL-2 inhibitor, or SN-38 of the present disclosure. Said combination of the antibody or functional part, derivative and/or analogue thereof that comprises a variable domain that can bind an extracellular part of EGFR of the present disclosure and the fluoropyrimidine, the platinum-based chemotherapeutic agent, the BCL-2 inhibitor, or SN-38 thus encompasses simultaneous, sequential or separate administration.

Hence, in certain aspects, the present disclosure provides an antibody or functional part, derivative and/or analogue thereof that comprises a variable domain that can bind an extracellular part of EGFR of the present disclosure for use in a method of treatment of a cancer, wherein the treatment further comprises administering of a fluoropyrimidine, a platinum-based chemotherapeutic agent, a BCL-2 inhibitor, or SN-38, wherein optionally said antibody or functional part, derivative and/or analogue thereof is administered simultaneously, sequentially or separately with said fluoropyrimidine, platinum-based chemotherapeutic agent, BCL-2 inhibitor, SN-38.

Hence, in certain aspects, the present disclosure provides a method of treatment of a subject having a cancer, comprising administering to the subject an effective amount of a fluoropyrimidine, a platinum-based chemotherapeutic agent, a BCL-2 inhibitor, or SN-38 and an antibody or functional part, derivative and/or analogue thereof that comprises a variable domain that can bind an extracellular part of EGFR of the present disclosure, wherein optionally said antibody or functional part, derivative and/or analogue thereof is administered simultaneously, sequentially or separately with said fluoropyrimidine, platinum-based chemotherapeutic agent, BCL-2 inhibitor, or SN-38.

Hence, in certain aspects, the present disclosure provides the use of an antibody or functional part, derivative and/or analogue thereof that comprises a variable domain that can bind an extracellular part of EGFR of the present disclosure and a fluoropyrimidine, a platinum-based chemotherapeutic agent, a BCL-2 inhibitor, or SN-38 in the manufacture of a medicament for the treatment of a cancer, wherein optionally said antibody or functional part, derivative and/or analogue thereof is administered simultaneously, sequentially or separately with said fluoropyrimidine, platinum-based chemotherapeutic agent, BCL-2 inhibitor, or SN-38. In certain aspects, said antibody or functional part, derivative and/or analogue thereof is administered prior to, simultaneously with, or after administration of said fluoropyrimidine, platinum-based chemotherapeutic agent, BCL-2 inhibitor, or SN-38.

In certain aspects, the antibody or functional part, derivative and/or analogue thereof that comprises a variable domain that can bind an extracellular part of EGFR of the present disclosure is for use in the manufacture of a medicament for the treatment of a cancer and the fluoropyrimidine, platinum-based chemotherapeutic agent, BCL-2 inhibitor, or SN-38 are for use in the manufacture of a medicament for the treatment of said cancer, wherein optionally said antibody or functional part, derivative and/or analogue thereof is administered simultaneously, sequentially or separately with said fluoropyrimidine, platinum-based chemotherapeutic agent, BCL-2 inhibitor, or SN-38. Optionally, said antibody or functional part, derivative and/or analogue thereof is administered prior to, simultaneously with, or after administration of said fluoropyrimidine, platinum-based chemotherapeutic agent, BCL-2 inhibitor, or SN-38.

### Definitions

In order that the present description may be more readily understood, certain terms are defined herein as follows. Additional definitions may be set forth throughout the detailed description where deemed required.

As used herein, the singular forms “a”, “an” and “the” include plural referents. Use of the term “comprising” “having” “including” as well as other forms, such as “comprise”, “comprises”, “comprised”, “has”, “have”, “had”, “include”, “includes”, and “included”, is not limiting.

The term “antibody” as used herein means a proteinaceous molecule belonging to the immunoglobulin class of proteins, containing one or more domains that bind an epitope on an antigen, where such domains are or derived from or share sequence homology with the variable region of an antibody. Antibodies are typically made of basic structural units, each with two heavy chains and two light chains. An antibody according to the present disclosure is not limited to any particular format or method of producing it.

A “bispecific antibody” is an antibody as described herein wherein one domain of the antibody binds to a first antigen whereas a second domain of the antibody binds to a second antigen, wherein said first and second antigens are not identical, or where one domain binds a first epitope on an antigen, whereas a second domain binds to a second epitope on the antigen. The term “bispecific antibody” also encompasses antibodies wherein one heavy chain variable region/light chain variable region (VH/VL) combination binds a first antigen or epitope on an antigen and a second VH/VL combination that binds a second antigen or epitope on the antigen. The term further includes antibodies wherein VH is capable of specifically recognizing a first antigen and the VL, paired with the VH in an immunoglobulin variable region, is capable of specifically recognizing a second antigen. The resulting VH/VL pair will bind either antigen 1 or antigen 2. Such so called “two-in-one antibodies”, are described in for instance WO 2008/027236, WO 2010/108127 and Schaefer et al (Cancer Cell 20, 472-486, October 2011). A bispecific antibody according to the present disclosure is not limited to any particular bispecific format or method of producing it.

The term ‘common light chain’ as used herein refers to the two light chains (or the VL part thereof) in the bispecific antibody. The two light chains (or the VL part thereof) may be identical or have some amino acid sequence differences while the binding specificity of the full-length antibody is not affected. The terms ‘common light chain’, ‘common VL’, ‘single light chain’, ‘single VL’, with or without the addition of the term ‘rearranged’ are all used herein interchangeably. “Common” also refers to functional equivalents of the light chain of which the amino acid sequence is not identical. Many variants of said light chain exist wherein mutations (deletions, substitutions, insertions and/or additions) are present that do not influence the formation of functional binding regions. In certain aspects, the light chain of the present disclosure can also be a light chain as specified herein, having from 0 to 10 amino acid insertions, deletions, substitutions, additions or a combination thereof. In certain aspects, the light chain of the present disclosure can also be a light chain as specified herein, having from 0 to 5 amino acid insertions, deletions, substitutions, additions or a combination thereof. It is for instance within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like.

As used herein, “to comprise” and its conjugations is used in its non-limiting sense to mean that items following the word are included, but items not specifically mentioned are not excluded. In addition, the verb “to consist” may be replaced by “to consist essentially of” meaning that a compound or adjunct compound as defined herein may comprise additional component(s) than the ones specifically identified, said additional component(s) not altering the unique characteristic of the disclosure.

A “derivative of an antibody” is a protein that but for the CDR regions, deviates from the amino acid sequence of a natural antibody in at most 20 amino acids. A derivative of an antibody as disclosed herein is an antibody that deviates from said amino acid sequence in at most 20 amino acids. The functional part, derivative and/or analogue maintains the binding specificity of the (bispecific) antibody. An “analogue of an antibody” is a protein that may be different in structure, format or origin but maintains the binding specificity of the antibody it is an analogue of. “Percent (%) identity” as referring to nucleic acid or amino acid sequences herein is defined as the percentage of residues in a candidate sequence that are identical with the residues in a selected sequence, after aligning the sequences for optimal comparison purposes. The percent sequence identity comparing nucleic acid sequences is determined using the AlignX application of the Vector NTI Advance© 11.5.2 software using the default settings, which employ a modified ClustalW algorithm (Thompson, J. D., Higgins, D. G., and Gibson T. J., (1994) Nucl. Acid Res. 22(22): 4673-4680), the swgapdnamt score matrix, a gap opening penalty of 15 and a gap extension penalty of 6.66. Amino acid sequences are aligned with the AlignX application of the Vector NTI Advance© 11.5.2 software using default settings, which employ a modified ClustalW algorithm (Thompson, J. D., Higgins, D. G., and Gibson T. J., (1994) Nucl. Acid Res. 22(22): 4673-4680), the blosum62mt2 score matrix, a gap opening penalty of 10 and a gap extension penalty of 0.1.

As an antibody typically recognizes an epitope of an antigen, and such an epitope may be present in other compounds as well, antibodies according to the present disclosure that “specifically recognize” an antigen, for example, EGFR or LGR5, may recognize other compounds as well, if such other compounds contain the same kind of epitope. Hence, the terms “specifically recognizes” with respect to an antigen and antibody interaction does not exclude binding of the antibodies to other compounds that contain the same kind of epitope.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein (so-called linear and conformational epitopes). Epitopes formed from contiguous, linear amino acids are typically retained on exposure to denaturing solvents, whereas epitopes formed by tertiary folding, conformation are typically lost on treatment with denaturing solvents. An epitope may typically include 3, 4, 5, 6, 7, 8, 9, 10, 11, 12, 13, 14 or amino acids in a unique spatial conformation.

As used herein, the terms “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer).

The words cancer and tumor are used herein and typically both refer to cancer, unless otherwise specifically stated.

The terms “treat,” “treating,” and “treatment,” as used herein, refer to any type of intervention or process performed on, or administering an active agent or combination of active agents to the subject with the objective of reversing, alleviating, ameliorating, inhibiting, or slowing down or preventing the progression, development, severity or recurrence of a symptom, complication, condition or biochemical indicia associated with a disease.

As used herein “effective treatment”” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, decrease the tumor size, decrease the presence of circulating tumor cells, reduce or prevent metastases of a tumor, slow or arrest tumor growth and/or prevent or delay tumor recurrence or relapse.

The term “effective amount” or “therapeutically effective amount” refers to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In terms of tumor development, an effective amount is an amount sufficient to delay tumor development. In terms of tumor recurrence, an effective amount is an amount sufficient to prevent or delay tumor recurrence. An effective amount can be administered in one or more administrations. The effective amount of the agent or combination of agents may: (i) reduce the number of cancer cells; (ii) reduce tumor size; (iii) inhibit, retard, slow to some extent and may stop cancer cell infiltration into peripheral organs; (iv) inhibit tumor metastasis; (v) inhibit tumor growth; (vi) prevent or delay occurrence and/or recurrence of tumor; and/or (vii) relieve to some extent one or more of the symptoms associated with the cancer. In one aspect, an “effective amount” is the amount of an antibody and a fluoropyrimidine, a platinum-based chemotherapeutic agent, a BCL-2 inhibitor, or SN-38, as disclosed herein, in combination, to affect a decrease in a cancer (for example a decrease in the number of cancer cells); slowing of progression of a cancer or prevent regrowth or recurrence of the cancer. As mentioned before herein, the antibody or functional part, derivative and/or analogue thereof that binds EGFR or binds EGFR and LGR5 of the present disclosure, and a fluoropyrimidine, a platinum-based chemotherapeutic agent, a BCL-2 inhibitor, or SN-38, in combination, is also referred herein to as a “therapeutic agent”. In certain aspects, the effective amount of the antibody or functional part, derivative and/or analogue thereof that binds EGFR or binds EGFR and LGR5 of the present disclosure herein is a flat dose of 1500 mg administered on a biweekly basis to a subject having a cancer of the present disclosure.

The term “flat dose” herein refers to a dosing regimen wherein a subject is administered with a fixed amount of a therapeutic substance over multiple administrations, independent of body weight of the subject. Flat dosing is typically abbreviated with qnw, wherein n is an integer indicating the interval and w is week. For instance, a q2w flat dose administration regimen of 1500 mg antibody means a fixed amount of 1500 mg antibody is administered each 2 weeks. Herein, in certain aspects, the therapeutic substance is an antibody binding EGFR or EGFR and LGR5 that is administered with a q2w dosing regimen of 1500 mg. In certain aspects, the subject has been administered at least 3 q2w flat dosages of 1500 mg. In certain aspects, said administration is at least 4 dosages or more and may last until the patient shows sufficient clinical or radiological progression.

The flat dose may be premedicated, meaning medication is administered to the subject prior to being administered the antibody of the present disclosure. In certain aspects, the flat dose of 1500 mg antibody is premedicated with an antihistamine, pain reducing medication, fever reducing medication and/or anti-inflammatory medication.

An effective amount of the combination therapy is administered according to the methods described herein in “an effective regimen” which refers to a combination of an antibody or functional part, derivative and/or analogue thereof of the present disclosure and a fluoropyrimidine, a platinum-based chemotherapeutic agent, a BCL-2 inhibitor, or SN-38, wherein the order of administration, amount dosed and dosage frequency is adequate to effect treatment.

As used herein, the terms “synergy”, “therapeutic synergy”, and “synergistic effect” refer to a phenomenon where treatment of patients with a combination of therapeutic agents (e.g., an EGFR/LGR5 binding antibody such as petosemtamab, in combination with a fluoropyrimidine, a platinum-based chemotherapeutic agent, a BCL-2 inhibitor, or SN-38) manifests a therapeutically superior outcome to the outcome achieved by each individual constituent of the combination when used alone (see, e.g., T. H. Corbett et al., 1982, Cancer Treatment Reports, 66, 1187). In this context a therapeutically superior outcome includes one or more of the following (a) an increase in therapeutic response that is greater than either or both of the separate effects of each agent alone at the same dose as in the combination; (b) a decrease in the dose of one or more agents in the combination without a decrease in therapeutic efficacy; (c) a decrease in the incidence of adverse events while receiving a therapeutic benefit that is equal to or greater than the monotherapy of each agent at the same dose as in the combination, (d) a reduction in dose-limiting toxicities while receiving a therapeutic benefit that is greater than the monotherapy of each agent; (e) a delay or minimization of the induction of drug resistance. In organoid models, a combination, used at its maximum tolerated dose, in which each of the constituents will be present at a dose generally not exceeding their individual maximum tolerated dose, manifests therapeutic synergy when decrease in organoid growth achieved by administration of the combination is greater than the value of the decrease in organoid growth of the best constituent when the constituent is administered alone.

All documents and references, including Genbank entries, patents and published patent applications, and websites, described herein are each expressly incorporated herein by reference to the same extent as if were written in this document in full or in part.

For the purpose of clarity and a concise description features are described herein as part of the same or separate parts of the disclosure, however, it will be appreciated that the scope of the disclosure may include preferred aspects having combinations of all or some of the features described.

The disclosure is now described by reference to the following examples, which are illustrative only, and are not intended to limit the present disclosure. While the disclosure has been described in detail and with reference to specific aspects thereof. it will be apparent to one of skill in the art that various changes and modifications can be made thereto without departing from the spirit and scope thereof.

### List of Clauses

1. An antibody or functional part, derivative and/or analogue thereof that comprises a variable domain that binds an extracellular part of EGFR, for use in the treatment of a cancer in a subject, wherein the treatment further comprises administering of a fluoropyrimidine.
2. A method of treating cancer in a subject, the method comprising administering an effective amount of an antibody or functional part, derivative and/or analogue thereof that comprises a variable domain that binds an extracellular part of EGFR, and a fluoropyrimidine to the subject.
3. The antibody or functional part, derivative and/or analogue thereof, or the method of clause 1 or 2, wherein the fluoropyrimidine is fluorouracil.
4. The antibody of functional, part, derivative and/or analogue thereof, or the method of any one of the preceding clauses, wherein fluorouracil is administered in a range of 200-3000 mg/m² intravenously.
5. The antibody or functional part, derivative and/or analogue thereof, or the method of any one of the preceding clauses, wherein fluorouracil is administered at 400 mg/m² by intravenous bolus on Day 1, followed by 2400 mg/m² to 3000 mg/m² intravenously as a continuous infusion over 46 hours every two weeks.
6. The antibody or functional part, derivative and/or analogue thereof, or the method of any one of the preceding clauses, wherein fluorouracil is administered at 500 mg/m² by intravenous bolus on Days 1, 8, 15, 22, 29, and 36 in 8-week cycles.
7. The antibody or functional part, derivative and/or analogue thereof, or the method of any one of the preceding clauses, wherein the cancer has a mutation in one or more genes selected from APC, KRAS, NRAS, SMAD2, SMAD4, SOX9, PIK3CA or TP53.
8. An antibody or functional part, derivative and/or analogue thereof that comprises a variable domain that binds an extracellular part of EGFR, for use in the treatment of a cancer in a subject, wherein the treatment further comprises administering of a fluoropyrimidine and a thymidine phosphorylase inhibitor.
9. A method of treating cancer in a subject, the method comprising administering an effective amount of an antibody or functional part, derivative and/or analogue thereof that comprises a variable domain that binds an extracellular part of EGFR, a fluoropyrimidine and a thymidine phosphorylase inhibitor to the subject.
10. The antibody or functional part, derivative and/or analogue thereof, or the method of clause 8 or 9, wherein the fluoropyrimidine is trifluorothymidine and the thymidine phosphorylase inhibitor is tipiracil.
11. The antibody or functional part, derivative and/or analogue thereof, or the method of any one of the clauses 8-10, wherein the trifluorothymidine and tipiracil are administered at a molar ratio of 1:0.5 at 35-80 mg/m² twice daily.
12. The antibody or functional part, derivative and/or analogue thereof, or the method of any one of the clauses 8-11, wherein the cancer has a mutation in one or more genes selected from APC, FBXW7, KRAS, NRAS, SMAD2, SMAD3, SMAD4, SOX9, PIK3CA or TP53.
13. An antibody or functional part, derivative and/or analogue thereof that comprises a variable domain that binds an extracellular part of EGFR, for use in the treatment of a cancer in a subject, wherein the treatment further comprises administering of a platinum-based chemotherapeutic agent.
14. A method of treating cancer in a subject, the method comprising administering an effective amount of an antibody or functional part, derivative and/or analogue thereof that comprises a variable domain that binds an extracellular part of EGFR, and a platinum-based chemotherapeutic agent to the subject.
15. The antibody or functional part, derivative and/or analogue thereof, or the method of clause 13 or 14, wherein the platinum-based chemotherapeutic agent is oxaliplatin.
16. The antibody of functional, part, derivative and/or analogue thereof, or the method of any one of the clauses 13-15, wherein oxaliplatin is administered in a range of 65-130 mg/m².
17. The antibody of functional, part, derivative and/or analogue thereof, or the method of any one of the clauses 13-16, wherein oxaliplatin is administered at 85 mg/m² every two weeks.
18. The antibody or functional part, derivative and/or analogue thereof, or the method of any one of the clauses 13-17, wherein the cancer has a mutation in one or more genes selected from APC, FBXW7, KRAS, NRAS, SMAD2, SMAD4, SOX9, PIK3CA or TP53.
19. An antibody or functional part, derivative and/or analogue thereof that comprises a variable domain that binds an extracellular part of EGFR, for use in the treatment of a cancer in a subject, wherein the treatment further comprises administering of a BCL-2 inhibitor.
20. A method of treating cancer in a subject, the method comprising administering an effective amount of an antibody or functional part, derivative and/or analogue thereof that comprises a variable domain that binds an extracellular part of EGFR, and a BCL-2 inhibitor to the subject.
21. The antibody or functional part, derivative and/or analogue thereof, or the method of clause 19 or 20, wherein the BCL-2 inhibitor is venetoclax.
22. The antibody of functional, part, derivative and/or analogue thereof, or the method of any one of the clauses 19-21, wherein venetoclax is administered in a range of 20-1200 mg/m² daily.
23. The antibody or functional part, derivative and/or analogue thereof, or the method of any one of the clauses 19-22, wherein the cancer has a mutation in one or more genes selected from APC, KRAS, NRAS, SMAD2, SMAD4, SOX9, PIK3CA or TP53.
24. An antibody or functional part, derivative and/or analogue thereof that comprises a variable domain that binds an extracellular part of EGFR, for use in the treatment of a cancer in a subject, wherein the treatment further comprises administering of SN-38.
25. A method of treating cancer in a subject, the method comprising administering an effective amount of an antibody or functional part, derivative and/or analogue thereof that comprises a variable domain that binds an extracellular part of EGFR, and SN-38 to the subject.
26. The antibody or functional part, derivative and/or analogue thereof, or the method of clause 24 or 25, wherein the cancer has a mutation in one or more genes selected from APC, FBXW7, KRAS, NRAS, PIK3CA, SMAD2, SMAD3, SMAD4, SOX9, TCF7L2, and TP53.
27. An antibody or functional part, derivative and/or analogue thereof that comprises a variable domain that binds an extracellular part of EGFR, for use in the treatment of a cancer in a subject, wherein treatment further comprises administering of oxaliplatin, folinic acid and fluorouracil.
28. A method of treating cancer in a subject, the method comprising administering an effective amount of an antibody or functional part, derivative and/or analogue thereof that comprises a variable domain that binds an extracellular part of EGFR, and oxaliplatin, folinic acid and fluorouracil to the subject.
29. The antibody or functional part, derivative and/or analogue thereof, or the method of clause 27 or 28, wherein oxaliplatin is administered at 50-200 mg/m² together with folinic acid at 200-600 mg/m² intravenously, followed by a fluorouracil at 1200-3600 mg/m² intravenously.
30. An antibody or functional part, derivative and/or analogue thereof that comprises a variable domain that binds an extracellular part of EGFR, for use in the treatment of a cancer in a subject, wherein the treatment further comprises administering of irinotecan, folinic acid and fluorouracil.
31. A method of treating cancer in a subject, the method comprising administering an effective amount of an antibody or functional part, derivative and/or analogue thereof that comprises a variable domain that binds an extracellular part of EGFR, and irinotecan, folinic acid and fluorouracil to the subject.
32. The antibody or functional part, derivative and/or analogue thereof, or the method of clause 30 or 31, wherein irinotecan is administered at 180 mg/m² together with folinic acid at 200-400 mg/m² intravenously, followed by fluorouracil at 400-2400 mg/m² intravenously.
33. The antibody of functional, part, derivative and/or analogue thereof, or the method of any one of the clauses 27-32, wherein the cancer has a mutation in one or more genes selected from APC, KRAS, NRAS, SMAD2, SMAD4, or SOX9.
34. The antibody or functional part, derivative and/or analogue thereof, or the method of any one of the preceding clauses, wherein the cancer is an adenocarcinoma or a squamous cell carcinoma.
35. The antibody or functional part, derivative and/or analogue thereof, or the method of any one of the preceding clauses, wherein the cancer is a head and neck cancer, gastric, esophageal, gastro-esophageal-junction cancer, non-small cell lung cancer or colorectal cancer.
36. The antibody or functional part, derivative and/or analogue thereof, or the method of any one of the preceding clauses, wherein the cancer is a colorectal cancer.
37. The antibody or functional part, derivative and/or analogue thereof, or the method of any one of the preceding clauses, wherein the antibody is ADCC enhanced.
38. The antibody or functional part, derivative and/or analogue thereof, or the method of any one of the preceding clauses, wherein the antibody is afucosylated.
39. The antibody or functional part, derivative and/or analogue thereof, or the method of any one of the preceding clauses, wherein the antibody is a multispecific antibody.
40. The antibody or functional part, derivative and/or analogue thereof, or the method of any one of the preceding clauses, wherein the antibody is a bispecific antibody.
41. The antibody or functional part, derivative and/or analogue thereof, or the method of any one of the preceding clauses, wherein the variable domain that binds an extracellular part of EGFR is a heavy chain variable region that comprises
   at least the CDR3 sequence of the VH of MF3370; MF3755; MF4280 or MF4289 as depicted in FIG. 3 or a CDR3 sequence that differs in at most three, two, or one amino acid from the CDR3 sequence of the VH of MF3370; MF3755; MF4280 or MF4289 as depicted in FIG. 3; or
   at least the CDR1, CDR2 and CDR3 sequences of the VH of MF3370; MF3755; MF4280 or MF4289 as depicted in FIG. 3; or the CDR1, CDR2 and CDR3 sequences of the VH of MF3370; MF3755; MF4280 or MF4289 as depicted in FIG. 3 with at most three, two, or one amino acid substitutions; or
   the amino acid sequence of the VH chain of MF3370; MF3755; MF4280 or MF4289 as depicted in FIG. 3; or
   the amino acid sequence of the VH chain of MF3370; MF3755; MF4280 or MF4289 depicted in FIG. 3 having at most 15, or 1, 2, 3, 4, 5, 6, 7, 8, 9 or 10 amino acid insertions, deletions, substitutions or a combination thereof with respect to the VH chain of MF3370; MF3755; MF4280 or MF4289.
42. The antibody or functional part, derivative and/or analogue thereof, or the method of any one of the preceding clauses, wherein the antibody comprises a second variable domain that does not bind EGFR.
43. The antibody or functional part, derivative and/or analogue thereof, or the method of any one of the preceding clauses, wherein the antibody comprises a second variable domain that binds LGR5.
44. The antibody or functional part, derivative and/or analogue thereof, or the method of any one of the preceding clauses, wherein the variable domain that binds LGR5 binds an epitope that is located within amino acid residues 21-118 of the human LGR5 sequence depicted in FIG. 1.
45. The antibody or functional part, derivative and/or analogue thereof, or the method of any one of the preceding clauses, wherein the variable domain that binds LGR5 is a heavy chain variable region that comprises
   at least the CDR3 sequence of the VH of MF5790; MF5803; MF5805; MF5808; MF5809; MF5814; MF5816; MF5817; or MF5818 as depicted in FIG. 3 or a CDR3 sequence that differs in at most three, two, or one amino acid from a CDR3 sequence of the VH of MF5790; MF5803; MF5805; MF5808; MF5809; MF5814; MF5816; MF5817; or MF5818 as depicted in FIG. 3; or
   at least the CDR1, CDR2 and CDR3 sequences of the VH of MF5790; MF5803; MF5805; MF5808; MF5809; MF5814; MF5816; MF5817; or MF5818 as depicted in FIG. 3;
   or the CDR1, CDR2 and CDR3 sequences of the VH of MF5790; MF5803; MF5805; MF5808; MF5809; MF5814; MF5816; MF5817; or MF5818 as depicted in FIG. 3 with at most three, two, or one amino acid substitutions; or
   the sequence of the VH chain of MF5790; MF5803; MF5805; MF5808; MF5809; MF5814; MF5816; MF5817; or MF5818 as depicted in FIG. 3; or
   the amino acid sequence of the VH chain of MF5790; MF5803; MF5805; MF5808; MF5809; MF5814; MF5816; MF5817; or MF5818 as depicted in FIG. 3 having at most 15, or 1, 2, 3, 4, 5, 6, 7, 8, 9 or 10 amino acid insertions, deletions, substitutions or a combination thereof with respect to the VH chain of MF5790; MF5803; MF5805; MF5808; MF5809; MF5814; MF5816; MF5817; or MF5818.
46. The antibody or functional part, derivative and/or analogue thereof, or the use or the method of any one of the preceding clauses, wherein the variable domain that binds an extracellular part of EGFR comprises a heavy chain variable region comprising the CDR1, CDR2 and CDR3 sequences of a variable region selected from the group consisting of MF3370; MF3755; MF4280 or MF4289 as depicted in FIG. 3 and wherein the variable domain that binds an extracellular part of LGR5 comprises a heavy chain variable region comprising the CDR1, CDR2 and CDR3 sequences of a variable region selected from the group consisting of MF5790; MF5803; MF5805; MF5808; MF5809; MF5814; MF5816; MF5817; or MF5818 as depicted in FIG. 3.
47. The antibody or functional part, derivative and/or analogue thereof, or the use or the method of any one of the preceding clauses, wherein the variable domain that binds an extracellular part of EGFR comprises a heavy chain variable region comprising the CDR1, CDR2 and CDR3 sequences of the variable region of MF3755 as depicted in FIG. 3 and wherein the variable domain that binds an extracellular part of LGR5 comprises a heavy chain variable region comprising the CDR1, CDR2 and CDR3 sequences of the variable region of MF5816 as depicted in FIG. 3.
48. The antibody or functional part, derivative and/or analogue thereof, or the use or the method of any one of the preceding clauses, wherein a VH chain of the variable domain that binds EGFR comprises the amino acid sequence of VH chain MF3370; MF3755; MF4280 or MF4289 as depicted in FIG. 3; or the amino acid sequence of VH chain MF3370; MF3755; MF4280 or MF4289 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH; and wherein a VH chain of the variable domain that binds LGR5 comprises the amino acid sequence of VH chain MF5790; MF5803; MF5805; MF5808; MF5809; MF5814; MF5816; MF5817; or MF5818 as depicted in FIG. 3; or the amino acid sequence of VH chain MF5790; MF5803; MF5805; MF5808; MF5809; MF5814; MF5816; MF5817; or MF5818 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH, preferably wherein the amino acid insertions, deletions and substitutions are not present in the CDR1, CDR2 and CDR3 light chain variable regions. 49. The antibody or functional part, derivative and/or analogue thereof, or the use or the method of any one of the preceding clauses, wherein a VH chain of the variable domain that binds EGFR comprises the amino acid sequence of VH chain MF3755 as depicted in FIG. 3; or the amino acid sequence of VH chain MF3755 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH; and wherein a VH chain of the variable domain that binds LGR5 comprises the amino acid sequence of VH chain MF5816 as depicted in FIG. 3; or the amino acid sequence of VH chain MF5816 as depicted in FIG. 3 having at most 15, preferably not more than 10, 9, 8, 7, 6, 5, 4, 3, 2, 1 and preferably having not more than 5, 4, 3, 2 or 1 amino acid modifications, including insertions, deletions, substitutions or a combination thereof with respect said VH, preferably wherein the amino acid insertions, deletions and substitutions are not present in the CDR1, CDR2 and CDR3 light chain variable regions.
50. The antibody or functional part, derivative and/or analogue thereof, or the use or the method of any one of the preceding clauses, wherein a VH chain of the variable domain that binds EGFR comprises the amino acid sequence of VH chain MF3370; MF3755; MF4280 or MF4289 as depicted in FIG. 3; and wherein a VH chain of the variable domain that binds LGR5 comprises the amino acid sequence of VH chain MF5790; MF5803; MF5805; MF5808; MF5809; MF5814; MF5816; MF5817; or MF5818 as depicted in FIG. 3.
51. The antibody or functional part, derivative and/or analogue thereof, or the use or the method of any one of the preceding clauses, wherein a VH chain of the variable domain that binds EGFR comprises the amino acid sequence of VH chain MF3755 as depicted in FIG. 3; and wherein a VH chain of the variable domain that binds LGR5 comprises the amino acid sequence of VH chain MF5816 as depicted in FIG. 3.
52. The antibody or functional part, derivative and/or analogue thereof, or the use or the method of any one of the preceding clauses, wherein both said variable domains that bind EGFR and that bind LGR5 comprise the CDR1, CDR2 and CDR3 regions of the light chain variable region as depicted in FIG. 4b.
53. The antibody or functional part, derivative and/or analogue thereof, or the use or the method of any one of the preceding clauses, wherein both said variable domains that bind EGFR and that bind LGR5 comprise the light chain variable region as depicted in FIG. 4b, which variable light chain region comprises from 0 to 10 amino acid insertions, deletions, substitutions, additions or a combination thereof, wherein the amino acid insertions, deletions and substitutions are not present in the CDR1, CDR2 and CDR3 light chain variable regions.
54. The antibody or functional part, derivative and/or analogue thereof, or the method of any one of the preceding clauses, wherein the antibody is petosemtamab.
55. The antibody or functional part, derivative and/or analogue thereof, or the method of any one of the preceding clauses, wherein the antibody is a monovalent antibody that does not comprise a second variable domain or wherein the antibody comprises said EGFR binding variable domain as the only variable domain.
56. The antibody or functional part, derivative and/or analogue thereof, or the method of any one of the preceding clauses, wherein the subject is a mammal, in particular a human.
57. The antibody or functional part, derivative and/or analogue thereof, or the method of any one of the preceding clauses, wherein said treatment comprises providing 1500 mg of the antibody or functional part, derivative and/or analogue thereof to the subject.
58. The antibody or functional part, derivative and/or analogue thereof, or the method of any one of the preceding clauses, wherein the subject to be treated has not received prior anti-cancer therapy.
59. The antibody or functional part, derivative and/or analogue thereof, or the method of any one of clauses 1-57, wherein the subject to be treated has progressed after prior anti-cancer therapy.
60. The antibody or functional part, derivative and/or analogue thereof, or the method of any one of the preceding clauses, wherein the prior anti-cancer therapy is chemotherapy, targeted anti-cancer therapy, immunotherapy or radiation therapy.
61. The antibody or functional part, derivative and/or analogue thereof, or the method of any one of the preceding clauses, wherein anti-cancer therapy is fluorouracil, a platinum-based chemotherapeutic agent, oxaliplatin, FOLFOX, FOLFIRI, TAS-102, trastuzumab, pembrolizumab, nivolumab, cetuximab or a combination thereof.
62. The antibody or functional part, derivative and/or analogue thereof, or the method of any one of the preceding clauses, wherein the subject has progressed after fluoropyrimidine-oxaliplatin-based chemotherapy with or without bevacizumab.
63. The antibody or functional part, derivative and/or analogue thereof, or the method of clause 62, wherein the subject is administered FOLFIRI.
64. The antibody or functional part, derivative and/or analogue thereof, or the method of any one of the preceding clauses, wherein the subject has progressed after fluoropyrimidine-irinotecan-based chemotherapy with or without bevacizumab.
65. The antibody or functional part, derivative and/or analogue thereof, or the method of clause 64, wherein the subject is administered FOLFOX.
66. The antibody or functional part, derivative and/or analogue thereof, or the method of any one of the preceding clauses, wherein said cancer is metastatic colorectal cancer (mCRC), such as an adenocarcinoma of the colon or rectum.
67. The antibody or functional part, derivative and/or analogue thereof, or the method of any one of the preceding clauses, wherein the antibody or functional part, derivative and/or analogue thereof and the fluoropyrimidine, the platinum-based chemotherapeutic agent, the BCL-2 inhibitor, or SN-38 are administered to the subject simultaneously, separately, or sequentially.
68. The method of treating cancer in a subject of any one of the preceding clauses, wherein the method comprises administering an effective amount of the antibody or functional part, derivative and/or analogue thereof, and an effective amount of the fluoropyrimidine, platinum-based chemotherapeutic agent, BCL-2 inhibitor, or SN-38, to the subject.
69. A pharmaceutical composition comprising an antibody or functional part, derivative and/or analogue thereof, as defined in any one of the preceding clauses; and a fluoropyrimidine, a platinum-based chemotherapeutic agent, a BCL-2 inhibitor, or SN-38.
70. The pharmaceutical composition according to clause 69 wherein the antibody or functional part, derivative and/or analogue thereof, is provided as a single formulation which includes the fluoropyrimidine, platinum-based chemotherapeutic agent, a BCL-2 inhibitor, or SN-38.
71. The pharmaceutical composition according to clause 69, wherein the antibody or functional part, derivative and/or analogue thereof, and the fluoropyrimidine, a platinum-based chemotherapeutic agent, a BCL-2 inhibitor, or SN-38 are provided in separate formulation.
72. A kit of parts comprising
   an antibody or functional part, derivative and/or analogue thereof, as defined in any one of the preceding clauses;
   a fluoropyrimidine, a platinum-based chemotherapeutic agent, a BCL-2 inhibitor, or SN-38; and
   instructions for use of said antibody or functional part, derivative and/or analogue thereof and instructions for use of said fluoropyrimidine, platinum-based chemotherapeutic agent, BCL-2 inhibitor, or SN-38.
73. The kit of parts, according to clause 72, wherein the instructions for use of the antibody or functional part, derivative/and or analogue thereof comprises instructions for dosing at 1500 mg.
74. The kit of parts according to clause 72 or 73, wherein the kit comprises instructions for use of the antibody or functional part, derivative and/or analogue thereof and fluoropyrimidine, platinum-based chemotherapeutic agent, BCL-2 inhibitor, or SN-38, in the treatment of adenocarcinoma or a squamous cell carcinoma.
75. The kit of parts according to any one of the clauses 72-74, wherein the kit comprises instructions for use of the antibody or functional part, derivative and/or analogue thereof and fluoropyrimidine, platinum-based chemotherapeutic agent, BCL-2 inhibitor, or SN-38, in the treatment of head and neck cancer, gastric, esophageal, gastro-esophageal-junction cancer, non-small cell lung cancer or colorectal cancer.
76. The kit of parts according to any one of the clauses 72-75, wherein the kit comprises instructions for use of the antibody or functional part, derivative and/or analogue thereof and fluoropyrimidine, platinum-based chemotherapeutic agent, BCL-2 inhibitor, or SN-38, in the treatment of colorectal cancer.
77. A combination of an antibody or functional part, derivative and/or analogue thereof as defined in any one of the preceding clauses, and a fluoropyrimidine, platinum-based chemotherapeutic agent, BCL-2 inhibitor, or SN-38, for use in the treatment of cancer in a subject in need thereof.
78. A combination of
   an antibody or functional part, derivative and/or analogue thereof, as defined in any one of the preceding clauses,
   instructions for use in the treatment of a cancer in a subject of said antibody and
   instructions for use in the treatment of a cancer in a subject of a fluoropyrimidine, a platinum-based chemotherapeutic agent, a BCL-2 inhibitor, or a SN-38, as defined in any one of the preceding clauses.
79. The combination of clause 78, wherein the instructions for use comprise the amount of the antibody or functional part, derivative and/or analogue thereof and/or the amount of the fluoropyrimidine, platinum-based chemotherapeutic agent, BCL-2 inhibitor, or SN-38 to be used, and/or the dosing interval and/or the cancer to be treated.
80. A combination of
   a fluoropyrimidine, a platinum-based chemotherapeutic agent, a BCL-2 inhibitor, or a SN-38, as defined in any one of the preceding clauses,
   instructions for use in the treatment of a cancer in a subject of a fluoropyrimidine, a platinum-based chemotherapeutic agent, a BCL-2 inhibitor, or a SN-38,
   instructions for use in the treatment of a cancer in a subject of an antibody or functional part, derivative and/or analogue thereof, as defined in any one of the preceding clauses.
81. The combination of clause 80, wherein the instructions for use comprise the amount of the antibody or functional part, derivative and/or analogue thereof and/or the amount of the fluoropyrimidine, platinum-based chemotherapeutic agent, BCL-2 inhibitor, or SN-38 to be used, and/or the dosing interval and/or the cancer to be treated.
82. Use of an antibody or functional part, derivative and/or analogue thereof as defined in any one of the preceding clauses and a fluoropyrimidine, a platinum-based chemotherapeutic agent, a BCL-2 inhibitor, or SN-38 as defined in any one of the preceding clauses, in the manufacture of one or more medicaments for the treatment of a cancer in a subject.
83. The use of clause 82, wherein the treatment comprises administering to the subject an effective amount of the antibody or functional part, derivative and/or analogue thereof and an effective amount of the fluoropyrimidine, the platinum-based chemotherapeutic agent, the BCL-2 inhibitor or the SN-38 to the subject.
84. The use of clause 82 or 83, wherein the treatment comprises administering the antibody or functional part, derivative and/or analogue thereof and the fluoropyrimidine, the platinum-based chemotherapeutic agent, the BCL-2 inhibitor or the SN-38 simultaneously, separately, or sequentially to the subject.

### EXAMPLES

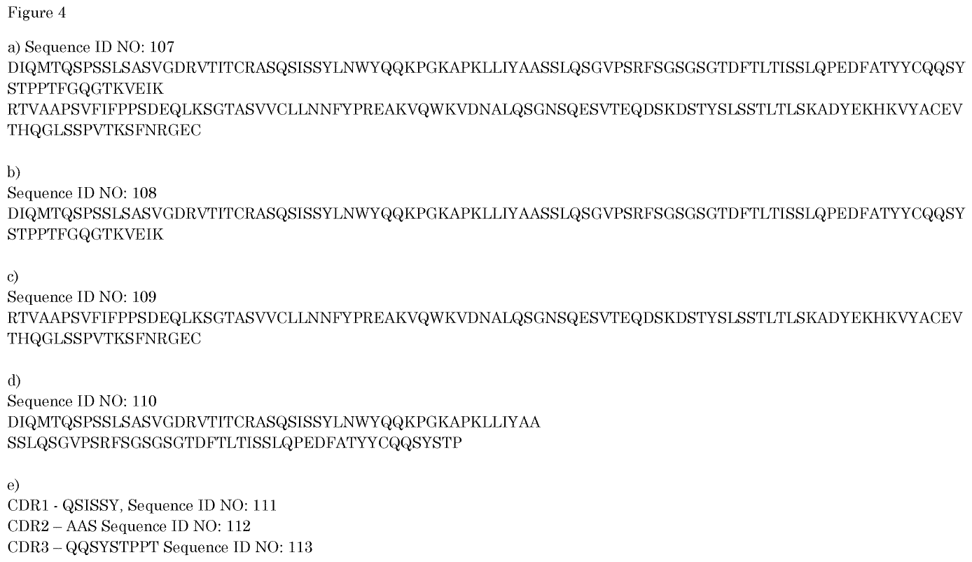

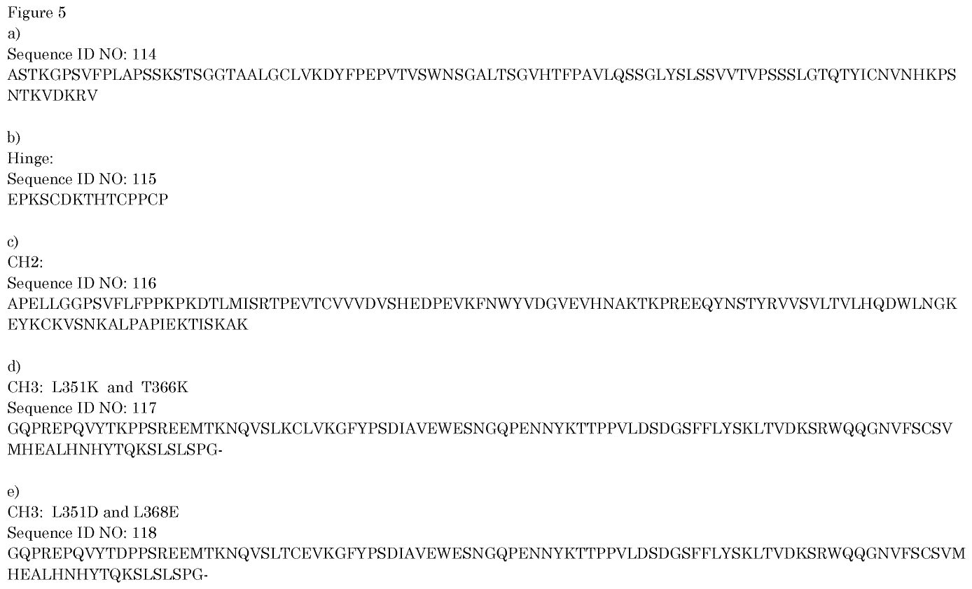

As used herein “MFXXXX” wherein X is independently a numeral 0-9, refers to a Fab comprising a variable domain wherein the VH has the amino acid sequence identified by the 4 digits depicted in FIG. 3. Unless otherwise indicated the light chain variable region of the variable domain typically has a sequence of FIG. 4b. The light chain in the examples has a sequence as depicted in FIG. 4a. “MFXXXX VH” refers to the amino acid sequence of the VH identified by the 4 digits. The MF further comprises a constant region of a light chain and a constant region of a heavy chain that normally interacts with a constant region of a light chain. The VH/variable region of the heavy chains differ and typically also the CH3 region, wherein one of the heavy chains has a KK mutation in its CH3 domain and the other has the complementing DE mutation in its CH3 domain (see for reference PCT/NL2013/050294 (published as WO2013/157954) and FIGS. 5d and 5e. Bispecific antibodies in the examples have an Fc tail with a KK/DE CH3 heterodimerization domain, a CH2 domain and a CH1 domain as indicated in FIG. 5, a common light chain as indicated in FIG. 4a and a VH as specified by the MF number. For example a bispecific antibody indicated by MF3755 xMF5816 has the above general sequences and a variable domain with a VH with the sequence of MF3755 and a variable domain with a VH with the sequence of MF5816.

The amino acid sequences of the various heavy chain variable regions (VH) are indicated in FIG. 3. Bispecific antibodies EGFR/LGR5, MF3755xMF5816, comprising heavy chain variable regions MF3755 and MF5816 and a common light chain and including modifications for enhanced ADCC from afucosylation, among other LGR5 and EGFR combinations as depicted in FIG. 3 have been shown to be effective in WO2017/069628.

### Example 1: Generation of Bispecific Antibodies

Bispecific antibodies were generated by transient co-transfection of two plasmids encoding IgG with different VH domains, using a proprietary CH3 engineering technology to ensure efficient heterodimerisation and formation of bispecific antibodies. The common light chain is also co-transfected in the same cell, either on the same plasmid or on another plasmid. In our applications (e.g. WO2013/157954 and WO2013/157953; incorporated herein by reference) we have disclosed methods and means for producing bispecific antibodies from a single cell, whereby means are provided that favor the formation of bispecific antibodies over the formation of monospecific antibodies. These methods can also be favorably employed in the present disclosure. Specifically, preferred mutations to produce essentially only bispecific full length IgG molecules are amino acid substitutions at positions 351 and 366, e.g. L351K and T366K (numbering according to EU numbering) in the first CH3 domain (the ‘KK-variant’ heavy chain) and amino acid substitutions at positions 351 and 368, e.g. L351D and L368E in the second CH3 domain (the ‘DE-variant’ heavy chain), or vice versa (see FIGS. 5d and 5e). It was previously demonstrated in the mentioned applications that the negatively charged DE-variant heavy chain and positively charged KK-variant heavy chain preferentially pair to form heterodimers (so-called ‘DEKK’ bispecific molecules). Homodimerization of DE-variant heavy chains (DE-DE homodimers) or KK-variant heavy chains (KK-KK homodimers) hardly occurs due to strong repulsion between the charged residues in the CH3-CH3 interface between identical heavy chains.

VH genes of variable domain that bind LGR5 described above were cloned into the vector encoding the positively charged CH3 domain. The VH genes of variable domain that bind EGFR such as those disclosed in WO 2015/130172 (incorporated herein by reference) were cloned into vector encoding the negatively charged CH3 domain. Suspension growth-adapted 293F Freestyle cells were cultivated in T125 flasks on a shaker plateau until a density of 3.0×10e6 cells/ml. Cells were seeded at a density of 0.3-0.5×10e6 viable cells/ml in each well of a 24-deep well plate. The cells were transiently transfected with a mix of two plasmids encoding different antibodies, cloned into the proprietary vector system. Seven days after transfection, the cellular supernatant was harvested and filtered through a 0.22 μM filter (Sartorius). The sterile supernatant was stored at 4° C. until purification of the antibodies.

### IgG Purification and Quantification

Purifications were performed under sterile conditions in filter plates using Protein-A affinity chromatography. First, the pH of the medium was adjusted to pH 8.0 and subsequently, IgG-containing supernatants were incubated with protein A Sepharose CL-4B beads (50% v/v) (Pierce) for 2 hrs at 25° C. on a shaking platform at 600 rpm. Next, the beads were harvested by filtration. Beads were washed twice with PBS pH 7.4. Bound IgG was then eluted at pH 3.0 with 0.1 M citrate buffer and the eluate was immediately neutralized using Tris pH 8.0. Buffer exchange was performed by centrifugation using multiscreen Ultracel 10 multiplates (Millipore). The samples were finally harvested in PBS pH7.4. The IgG concentration was measured using Octet. Protein samples were stored at 4° C.

To determine the amount of IgG purified, the concentration of antibody was determined by means of Octet analysis using protein-A biosensors (Forte-Bio, according to the supplier's recommendations) using total human IgG (Sigma Aldrich, cat. nr. I4506) as standard.

The following bispecific antibodies are suitable for use in this example and for use in the methods of the disclosure: MF3370xMF5790, MF3370xMF5803, MF3370xMF5805, MF3370xMF5808, MF3370xMF5809, MF3370xMF5814, MF3370xMF5816, MF3370xMF5817, MF3370xMF5818, MF3755xMF5790, MF3755xMF5803, MF3755xMF5805, MF3755xMF5808, MF3755xMF5809, MF3755xMF5814, MF3755xMF5816, MF3755xMF5817, MF3755xMF5818, MF4280xMF5790, MF4280xMF5803, MF4280xMF5805, MF4280xMF5808, MF4280xMF5809, MF4280xMF5814, MF4280xMF5816, MF4280xMF5817, MF4280xMF5818, MF4289xMF5790, MF4289xMF5803, MF4289xMF5805, MF4289xMF5808, MF4289xMF5809, MF4289xMF5814, MF4289xMF5816, MF4289xMF5817, and MF4289xMF5818. Each bispecific antibody comprises two VH as specified by the MF numbers capable of binding EGFR and LGR5 respectively, further comprises an Fc tail with a KK/DE CH3 heterodimerization domain as indicated by SEQ ID NO: 117 (FIG. 5d) and SEQ ID NO: 118 (FIG. 5e), respectively, a CH2 domain as indicated by SEQ ID NO: 116 (FIG. 5c), a hinge domain as indicated by SEQ ID NO: 115 (FIG. 5b) and a CH1 domain as indicated by SEQ ID NO: 114 (FIG. 5a), a common light chain as indicated by SEQ ID NO: 107 (FIG. 4a).

### Example 2: Materials and Methods

### Organoid Models:

Tumor organoid models were obtained from HUB organoids (see huborganoids.nl) and are as described in van de Wetering, M. et al. Prospective derivation of a living organoid biobank of colorectal cancer patients, Cell 161, 933-945 (2015). Mutations in components of the four main CRC driver pathways (WNT, RTK/RAS, TP53 and transforming growth factor-6) in each model are summarized in Table 4. Further details of the clinical-pathological features can be found in Herpers B. et al. Nat Cancer. 2022 April; 3(4):418-436.

TABLE 4
Organoid models

| | | | mono- | | |
|---|---|---|---|---|---|
| | | Bi-allelic | allelic | | |
| | Specimen | loss of | loss of | Gain of | Loss of |
| | type | function | function | function | heterozygosity |
| P18T | | APC | | | |
| C55T | Primary | | APC, | KRAS | NRAS, SMAD2, |
| | tumor | | SOX9 | | SMAD4 |
| C20T | Primary | TP53 | | | APC, NRAS, |
| | tumor | | | | SMAD2, |
| | | | | | SMAD4, |
| | | | | | FBXW7 |
| C82T | Primary | APC | TP53 | KRAS , | |
| | tumor | | | PIK3CA | |
| C82N | Tumor- | — | — | — | — |
| | adjacent | | | | |
| | healthy | | | | |
| | mucosa | | | | |
| C92T | Primary | APC, TP53 | SOX9; | | TCF7L2, NRAS, |
| | tumor | | GOF: | | SMAD2, |
| | | | KRAS, | | SMAD3, |
| | | | PIK3CA | | SMAD4 |
| C92N | Tumor- | — | — | — | — |
| | adjacent | | | | |
| | healthy | | | | |
| | mucosa | | | | |

Culture medium: The organoids were expanded and exposed in Matrigel Matrix Basement Membrane (Corning, Cat. no 354230) droplets in Organoid Culture Medium (OCM) and seeded in 384-wells plates (Greiner, Cat. no 781091) using a liquid handler. In brief, OCM contains RSP03 (BioTechne, 3500-RS/CF), Noggin (Peprotech, 120-10C), B27 (Thermo Fisher Scientific, Cat. no 17504001), Nicotinamide (Sigma Aldrich, Cat. no N0636), N-acetylcysteine (Sigma Aldrich, Cat. no A9165), SB-202190 (Sigma Aldrich, Cat. no S7067), Gastrin (Merck, Cat. no G9145) and A83-01 (Tocris, Cat. no 2939) in Advanced DMEM (Thermo Fisher Scientific, Cat. no 12634028) with Pen/Strep (Thermo Fisher Scientific, Cat. no 15140122), Hepes (Thermo Fisher Scientific, Cat. no 15630-056) and Glutamax (Thermo Fisher Scientific, Cat. no 35050038). OCM is supplemented with WNT3A-conditioned medium (OL-016_v2) for healthy colon organoid models. During passaging 10 μM Y27632 (RhoKi) (Bioconnect, Cat. no M1817) was used.

Colon organoids were seeded as 300 or 600 (model P18T) 10-20 cell stage cell clusters in a hydrogel in 384-wells plates. Upon 30 minutes gelation, medium was added. For colon tumoroids, organoid culture medium was used. For normal colon organoids, organoid culture medium supplemented with WNT3A was used. Growth factors were added to the culture medium at the indicated concentrations on the days of seeding. Bispecific antibodies were added to the medium with growth factors on the day of seeding. Compound exposures were performed on the day of seeding or 24 h after seeding. The organoids were exposed to the growth factors, antibodies and compounds for 5-8 days (dependent on the growth rate of the organoid models—normal organoids deplete the culture medium quicker than colon tumoroids). At the end of an experiment the organoids were fixed to stain the actin cytoskeleton and nuclei, to prepare for image-based analysis in Ominer® of 25-40 sections of 50 μm stepsize per z-stack, captured using a 4×objective (1080×1080 pixels) per well. The per well data were aggregated and the averages and standard deviations per morphological feature (>500 features) were recorded and processed.

Growth factors: EGF (Peprotech. Cat. no AF-100-15) was used at 10 ng/ml during the expansion of the tumoroid models and at 50 ng/ml for the healthy organoid models.

### Test Compounds:

Petosemtamab: 22.9 mg/ml (Lot: FB01701; Stored at 4° C.); PG2708p218: 2.5 mg/ml (Stored at 4° C.); 5-FU (F-2449): 10 mM (DMSO) (Lot: G1264; Stored at −20° C.); Oxaliplatin (15604985): 12.59 mM (PBS) (Lot: 211E0081; Stored at 4° C.); SN-38 (S4908): 10 mM (DMSO) (Lot: 86639-52-3-02; Stored at −20° C.); TAS-102 (S8539): 10 mM (DMSO) (Lot: S853901; Stored at −20° C.); Venetoclax (S8048): 10 mM (DMSO) (Lot: S804806; Stored at −20° C.); Staurosporine (HY-15141): 10 mM (DMSO) (Lot: CS2716; Stored at −20° C.).

### Image Analysis

After incubation, the organoids were fixed and stained to visualize the nuclei and the actin cytoskeleton respectively. Upon washing in PBS the plates were imaged at 4× magnification in a Molecular Devices ImageXpress Micro XLS system at a 50 μm z-step size for 25-40 sections per well. The images were processed to quantify the shape, dimensions, location, number and intensity of each individual nucleus, organoid, lumen and epithelium within the organoids.

QC was run on the segmented images and on the data by bootstrapping the data of the positive and negative control groups to calculate the randomized Z′-factor and one-way ANOVA as a measure for data consistency. The aggregated data was normalized (z-score and percentage-of-control (POC) with DMSO+PBS+PG2708 as negative control references) and processed in TIBCO Spotfire® or in MS Excel to format the data for graphical visualization and curve fitting in GraphPad Prism v8.4.

### Example 3: Dose Range Testing of Individual Compounds

A dose range of petosemtamab, 5-FU, Oxaliplatin, SN-38, TAS-102 and Venetoclax was tested in 5 ng/ml EGF conditions to estimate the IC20, in preparation for combination studies in Experiment 4. The models that were tested are P18T, C55T, C82T, and C82N. PBS, DMSO and PG2708 negative control antibody were used as negative controls. 1 μM Staurosporine and Ong/ml EGF were used as positive controls. Compound exposure was on the day of seeding for organoid models C82N, C82T, C55T and P18T and exposure time was 8 days. Table 5 indicates the different concentrations tested for the compounds.

TABLE 5
Compound Concentrations tested

| Compound | Unit | Dose | | | | | | | |
|---|---|---|---|---|---|---|---|---|---|
| 5-FU | μM | 100 | 31.6 | 10 | 3.16 | 1 | 0.316 | 0.1 | 0.0316 |
| Oxaliplatin | μM | 31.6 | 10 | 3.16 | 1 | 0.316 | 0.1 | 0.0316 | 0.01 |
| Irinotecan | μM | 31.6 | 10 | 3.16 | 1 | 0.316 | 0.1 | 0.0316 | 0.01 |
| SN-38 | μM | 1000 | 316 | 100 | 31.6 | 10 | 3.16 | 1 | 0.316 |
| TAS-102 | μM | 10 | 3.16 | 1 | 0.316 | 0.1 | 0.0316 | 0.01 | 0.003 |
| Venetoclax | μM | 10 | 3.16 | 1 | 0.316 | 0.1 | 0.0316 | 0.01 | 0.003 |
| petosemtamab | μg/ml | 31.6 | 10 | 3.16 | 1 | 0.316 | 0.1 | 0.0316 | 0.01 |

The controls behaved as expected. All compounds reduced proliferation, increased cell death and reduced lumen formation in organoids with increasing compound doses. Based on the gathered results, IC20 for all the compounds was determined in preparation of Example 4. The IC20 values are summarized in Table 6.

TABLE 6
Summary of IC20 of individual compounds and proposed dose range

| IC20 table | P18T | | C55T | | C82N | | C82T | | Proposed dose range |
|---|---|---|---|---|---|---|---|---|---|
| 5-FU (μM) | 0.36 | (±0.11) | 0.42 | (±0.29) | 16.94 | (±23.53) | 2.73 | (±5.59) | 0.1-0.316-1-3.16-10 μM |
| SN-38 (nM) | 0.94 | (±0.56) | 2.51 | (±1.13) | 180.67 | (±260.23) | 1.85 | (±0.28) | 0.1-0.316-1-3.16-10 μM |
| Oxaliplatin | 4.82 | (±0.56) | 4.04 | (±0.98) | 7.82 | (±15.42) | 15.81 | (±9.54) | 0.316-1-3.16-10-31.6 μM |
| (μM) | | | | | | | | | |
| TAS-102 (μM) | 0.22 | (±0.09) | 0.92 | (±0.49) | 0.23 | (±0.07) | 1.44 | (±0.96) | 0.1-0.316-1-3.16-10 μM |
| Venetoclax | 2.05 | (±0.41) | 1.7 | (±0.33) | 7.69 | (±2.42) | 1.9 | (±0.64) | 0.1-0.316-1-3.16-10 μM |
| (μM) | | | | | | | | | |
| petosemtamab | 0.069 | (±0.017) | 0.034 | (±0.012) | 5.22 | (±2.43) | 1.03 | (±0.73) | 0.0316-0.1-0.316-1-3.16 μg/ml |
| (μg/ml) | | | | | | | | | |

### Example 4: Combination Studies of Petosemtamab and Individual Compounds

It was tested whether petosemtamab treatment would sensitize organoids towards 5-FU, Oxaliplatin, SN-38, TAS-102 or Venetoclax treatment in a 6×6 combination matrix in ng/ml EGF conditions in models P18T, C20T, C55T, C82T, C92T, and C92N. On the day of seeding the following combinations were added: Petosemtamab (0-0.031-0.1-0.316-1-3.162pg/ml); with 5-FU, TAS-102 or Venetoclax at 0-0.1-0.316-1-3.162-10 μM; or SN38 at 0-0.1-0.316-1-3.162-10 nM; or Oxaliplatin at 0-0.316-1-3.162-10-31.62 μM.

The antibody-drug combinations were incubated for 7 days before fixation to visualize the 3D actin and nucleus organization in the organoids. PBS, DMSO and PG2708 negative control antibody were used as negative controls. 1 μM Staurosporine and Ong/ml EGF were used as positive controls.

For each model and combinations, three features were evaluated: nucleus count, sum child count (total number of surviving nuclei), and total organoid size. Total organoid size is known to reduce upon growth inhibition and cell death induction.

The controls behaved as expected and were consistent across the experiments. All compound combinations reduced the outgrowth of organoids and increased cell death. Total organoid size was further used to evaluate synergism between the different combinations.

### Data Analysis:

Data was analyzed using the online tool SynergyFinder.org (Zheng, S. et. al. bioRxiv 2021.06.01.446564 (2021); Zheng S, et. al. SynergyFinder Plus: Toward Better Interpretation and Annotation of Drug Combination Screening Datasets. Genomics Proteomics Bioinformatics. 2022 Jan. 25: S1672-0229(22)00008-0). The SynergyFinder tool uses four methods: HSA, LOEWE, BLISS and ZIP to indicate the degree of interaction. Following SynergyFinder analysis, CI was calculated for each datapoint using the Chou-Talalay method (cf. Chou T. C. Drug combination using the Chou-Talalay method. Cancer research 70, 440-446 (2010)). CI was calculated for each datapoint using the maximum value of either the measured single agent effect or the value predicted by the model based on the effects of the single agents, divided by the actual measured effect using all four methods (ZIP, BLISS, LOEWE and HSA). The resulting synergy scores and CI scores are summarized for the different models in Table 7.

TABLE 7
Synergy scores and combination index (CI) scores for different
models for combination treatment of petosemtamab with
5-FU, Oxaliplatin, SN-38, TAS-102 or Venetoclax.

| | | | | | | | CI |
|---|---|---|---|---|---|---|---|
| Model | Drug1 | Drug2 | ZIP | HAS | Loewe | Bliss | score |
| C82T | petosemtamab | 5-FU | 4.90 | 3.35 | 2.97 | 4.84 | 0.572 |
| C55T | petosemtamab | 5-FU | 4.83 | 8.68 | 8.78 | 2.85 | 0.672 |
| C55T | petosemtamab | Oxaliplatin | 14.44 | 16.22 | 15.55 | 14.09 | 0.410 |
| C82T | petosemtamab | Oxaliplatin | 7.46 | 6.38 | 5.63 | 7.77 | 0.481 |
| C92N | petosemtamab | Oxaliplatin | 0.34 | 0.84 | 0.83 | 0.38 | 0.675 |
| C20T | petosemtamab | Oxaliplatin | 1.48 | 1.41 | 1.13 | 0.48 | 0.725 |
| P18T | petosemtamab | Oxaliplatin | 2.69 | 4.81 | 2.42 | 2.41 | 0.809 |
| C55T | petosemtamab | SN38 | 14.55 | 14.03 | 12.65 | 15.09 | 0.365 |
| C92N | petosemtamab | SN38 | 1.45 | 1.94 | 1.22 | 1.80 | 0.461 |
| C82T | petosemtamab | SN38 | 5.19 | 4.31 | 3.82 | 5.01 | 0.506 |
| C20T | petosemtamab | SN38 | 0.79 | 1.05 | 0.90 | 1.07 | 0.530 |
| C92T | petosemtamab | SN38 | 4.48 | 7.02 | 6.63 | 4.17 | 0.594 |
| C82T | petosemtamab | TAS-102 | 6.09 | 6.00 | 5.92 | 6.83 | 0.303 |
| C55T | petosemtamab | TAS-102 | 6.73 | 9.38 | 8.43 | 6.68 | 0.515 |
| C82T | petosemtamab | Venetoclax | 6.80 | 7.13 | 6.79 | 6.85 | 0.198 |
| C55T | petosemtamab | Venetoclax | 1.79 | 6.08 | 5.13 | 1.49 | 0.766 |
| C20T | petosemtamab | Venetoclax | −0.01 | 0.93 | 0.43 | 0.01 | 0.854 |

In sum, combination treatment with petosemtamab and each of the five tested standard of care drugs led to synergistic growth inhibition in the indicated models.

### Example 5: Combination Studies of Petosemtamab and Folfox/Folfiri

It was also tested whether petosemtamab combination treatment with FOLFOX and FOLFIRI promoted the efficiency of tumor regression. The individual components of FOLFOX and FOLFIRI combination drugs are described elsewhere herein. In patients, the first treatment is 2 hours IV Oxaliplatin at 85 mg/m². This correlates with a plasma Cmax of 1.61-2.2ug/ml or 4-5 uM. Next Leucovorin is provided IV in combination with Irinotecan for 90-120 minutes at 180 mg/m². 150 mg/m² Irinotecan leads to an approximate plasma Cmax of 2 μg/ml Irinotecan, which provides on average 36 ng/ml of its metabolite SN-38 (92 nM). Next 5-FU is provided as a bolus injection of 400 mg/m², leading to an average Cmax of 55ug/ml, followed by IV infusion over 46 hours of 2400 mg/m². Literature indicates that IV infusion over 3 days at 1750 mg/m² leads to an average plasma concentration of 4.6 uM (peak is 7.3 uM). Leucovorin or Folinic acid was used up to the concentration at which it could be dissolved (50pg/ml). This led to the following combinations:
5 μM 5-FU+5 μM Oxaliplatin+50pg/ml Folinic acid to mimic FOLFOX (5 μM) or
5 uM 5-FU+100 nM SN-38+50ug/ml Folinic acid to mimic FOLFIRI (5 uM).

Combinations were tested in model C55T in conditions containing 5 ng/ml EGF.

Synergy was calculated for the FOLFIRI and FOLFOX combination using total organoid size and the method as described in Example 4. The synergy scores and Combination Index (CI) are summarized in Table 8. Synergy scores and CI indicate that both FOLFIRI and FOLFOX have synergistic effects in combination with petosemtamab in the indicated model.

TABLE 8
Synergy scores and combination sensitivity scores calculated
in SynergyFinder for model C55T for combination treatment
of petosemtamab with FOLFOX or FOLFIRI.

| | | | | | | | | CI |
|---|---|---|---|---|---|---|---|---|
| | Model | Drug 1 | Drug 2 | ZIP | HAS | Loewe | Bliss | score |
| 1 | C55T | Petosemtamab | FOLFIRI | 3.69 | 7.68 | 5.62 | 4.05 | 0.49 |
| 2 | C55T | Petosemtamab | FOLFOX | 3.37 | 8.94 | 7.81 | 3.65 | 0.62 |

### Example 6: Clinical Study Protocol

### Study Design

This is a first in human phase 1/2 open-label multicenter study combining an initial dose escalation part with a dose expansion part the combination cohort for mCRC. The initial dose escalation part was completed, and the preliminary RP2D was established at 1500 mg Q2W.

A combination of petosemtamab and selected chemotherapeutic agents is evaluated in a dose expansion part of the study primarily aimed at treating mCRC.

Petosemtamab will be administered IV as a flat dose over an infusion period of 2 to approximately 6 h, Q2W, with 4-week cycles (28 days).

Patients will be assigned to a dose expansion treatment arm which includes a petosemtamab arm as a single agent and one arm using petosemtamab in combination with chemotherapeutic agents. In particular, a combination of petosemtamab with FOLFOX and in another study arm, a combination with FOLFIRI is tested.

Safety, PK, immunogenicity, and antitumor activity will be characterized in all cohort patients, and retrospective biomarker analyses including EGFR and LGR5 status will be performed.

### Study Population

### Inclusion Criteria

1. Signed informed consent form (ICF) before initiation of any study procedures.
2. Age ≥18 years at signing of ICF.
3. Histologically or cytologically confirmed solid tumors with evidence of metastatic or locally advanced disease not amenable to standard therapy with curative intent:
   Expansion cohorts: patients with locally advanced unresectable or metastatic disease for the following indications:
      Patients with mCRC receiving treatment as second line therapy are previously diagnosed with histologically or cytologically confirmed unresectable or metastatic adenocarcinoma of the colon or rectum. Patients are wildtype for RAS/RAF as determined using NGS on tumor tissue (primary or metastatic). The NGS data report is provided for Sponsor confirmation before enrolment. Patients are naive to prior anti-EGFR therapy. Radiographically confirmed disease progression has occurred during or within 6 months of prior 1L chemotherapy.
      Cohort to be treated with petosemtamab and FOLFIRI: patients have had only one prior chemotherapy regimen for the metastatic setting, consisting of 1L fluoropyrimidine-oxaliplatin-based chemotherapy with or without bevacizumab.
      Cohort to be treated with petosemtamab and FOLFOX: patients should have had only 1 prior chemotherapy regimen for the metastatic setting, consisting of 1L fluoropyrimidine-irinotecan-based chemotherapy with or without bevacizumab.
4. A baseline new tumor sample (formalin-fixed paraffin-embedded [FFPE]) from a metastatic or primary site. If the patient has an available tumor sample as an FFPE block with sufficient material (at least 20 slides with >20% tumor content) and has not received further anticancer treatment since sample collection, a new tumor biopsy at baseline is not necessary. Archival FFPE slides are not acceptable.
5. Measurable disease as defined by RECIST v1.1 by radiologic methods.
6. ECOG PS of 0 or 1.
7. Life expectancy ≥12 weeks, as per investigator.
8. Left ventricular ejection fraction (LVEF) 250% by echocardiogram (ECHO) or multigated acquisition (MUGA) scan.
9. Adequate organ function:
   Absolute neutrophil count (ANC) ≥1.5×10⁹/L
   Hemoglobin ≥9 g/dL
   Platelets ≥100×10⁹/L
   Serum magnesium, sodium, corrected total calcium, phosphate, and potassium within normal ranges (or corrected with supplements or appropriate treatment). If these electrolyte ranges are not within normal range despite corrective treatment, then the Sponsor should be consulted to confirm eligibility.
   Alanine aminotransferase (ALT), aspartate aminotransferase (AST) ≤2.5×upper limit of normal (ULN) and total bilirubin ≤1.5×ULN (unless due to known Gilbert's syndrome who are excluded if total bilirubin >3.0×ULN or direct bilirubin >1.5×ULN); in cases of liver involvement, ALT/AST ≤5×ULN and total bilirubin ≤2×ULN will be allowed, unless due to known Gilbert's syndrome when total bilirubin ≤3.0×ULN or direct bilirubin ≤1.5×ULN will be allowed.
   Serum creatinine ≤1.5×ULN or creatinine clearance(CrCl) ≥60 mL/min calculated according to the Cockroft and Gault formula or Modification of Diet in Renal Disease (MDRD) formula for patients aged >65 years.
   International normalized ratio (INR) or prothrombin time (PT)<1.5×ULN unless patient is receiving anticoagulant therapy and in therapeutic range of intended used anticoagulant.
   Activated partial thromboplastin time (APTT) or partial thromboplastin time (PTT) ≤1.5×ULN unless patient is receiving anticoagulant therapy and is in therapeutic range of intended used anticoagulant.

### Exclusion Criteria

1. Central nervous system metastases that are untreated or symptomatic, or require radiation, surgery, or continued steroid therapy to control symptoms within 14 days of study entry.
2. Known leptomeningeal involvement.
3. Participation in another clinical study or treatment with any investigational drug within 4 weeks prior to study entry.
4. Any systemic anticancer therapy within 4 weeks or 5 half-lives, whichever is longer, of the first dose of study treatment. For cytotoxic agents that have major delayed toxicity (eg, mitomycin C, nitrosoureas), or anticancer immunotherapies, a washout period of 6 weeks is required.
5. Requirement for immunosuppressive medication (eg, methotrexate, cyclophosphamide).
6. Major surgery or radiotherapy within 3 weeks of the first dose of study treatment. Patients who received prior radiotherapy to ≥25% of bone marrow are not eligible, irrespective of when it was received.
7. Persistent Grade >1 clinically significant toxicities related to prior antineoplastic therapies (except for alopecia); stable sensory neuropathy Grade ≤2 National Cancer Institute-Common Terminology Criteria for Adverse Events (NCI-CTCAE) v4.03/v5.0 or as current at the time of administration is allowed.
8. History of hypersensitivity reaction to any of the excipients of petosemtamab, human proteins, or any non-IMP treatment required for this study.
9. Uncontrolled hypertension (systolic blood pressure [BP]>150 mmHg and/or diastolic BP >100 mmHg) with appropriate treatment; unstable angina; history of congestive heart failure of Class II-IV New York Heart Association (NYHA) criteria, or serious cardiac arrhythmia requiring treatment (except atrial fibrillation, paroxysmal supraventricular tachycardia); or history of myocardial infarction within 6 months of study entry.
10. History of prior malignancies with the exception of excised cervical intraepithelial neoplasia or nonmelanoma skin cancer, or curatively treated cancer deemed at low risk for recurrence with no evidence of disease for ≥3 years, or a second primary malignancy.
11. Current dyspnea at rest of any origin, or other diseases requiring continuous oxygen therapy, including patients with a history of interstitial lung disease (ILD) (eg, pneumonitis or pulmonary fibrosis), or evidence of ILD on baseline chest computerized tomography (CT) scan.
12. Current serious illness or medical conditions including, but not limited to, uncontrolled active infection, clinically significant pulmonary, metabolic or psychiatric disorders.
13. Patients with known infectious diseases
14. Pregnant or breastfeeding patients; patients of childbearing potential must use highly effective contraception methods prior to study entry, for the duration of study participation, and for 6 months after the last dose of petosemtamab.
15. mCRC with a RAS/RAF mutation identified by local ctDNA or tumor test at screening, or identified as such in disease history, are not eligible for this study.
16. For the mCRC cohort; patients with an active inflammatory bowel disease, or other bowel disease causing chronic diarrhea (defined as NCI-CTCAE Grade ≥2), are not eligible for this study.
17. Patients with peripheral sensory neuropathy with functional impairment (defined as NCI CTCAE Grade ≥3) are not eligible for this study.

### Prescreening of Patients

Prescreening may be performed to exclude somatic mutations in RAS and RAF family genes (i.e. KRAS, NRAS, HRAS, BRAF, ARAF, RAF1 wildtype).

Prescreening of mCRC patients in the second line setting to be treated in combination with FOLFIRI or FOLFOX: may be applied to determine eligibility, a report from a validated tumor NGS assay is to confirm that no RAS/RAF or other relevant mutation was detected in the patient's tumor. This report may be from any time during the natural history of disease. RAS/RAF status must be known for the patient to be considered for this cohort.

### Petosemtamab Administration

Petosemtamab is administered by IV infusion Q2W using 1500 mg Q2W dosing regimen. Infusions are administered IV over approximately 6 h for the Cycle 1 Day 1 infusion. Subsequent infusions after Cycle 1 Day 1 can be reduced to 2 h (±15 min) at the investigator's discretion and in the absence of IRRs. Alternatively, petosemtamab is used at 1100 mg flat dose Q2W or administrated in an amount which achieves human receptor target engagement for both EGFR and LGR5 of at least 90%, at least 95%, at least 99% across relevant body weights for a statistically significant number of subjects. Said target engagement of 90% may be achieved using a flat dose of about 1000 mg Q2W. Said amount of 95% may be achieved using a flat dose of about 1100 to about 1200 mg Q2W.

A cycle is considered 4 weeks. For each patient, a 1 h observation period will be implemented following the end of infusion (EOI).

### Treatment Duration

Study treatment will be administered until confirmed progressive disease (PD) per independent review (as per RECIST v1.1), unacceptable toxicity, withdrawal of patient consent, patient noncompliance, investigator decision (eg, clinical deterioration), or petosemtamab interruption >6 consecutive weeks. All patients enter the survival follow-up phase and will continue to be followed for survival status until the end of the study, and all patients have had the opportunity to be followed for ≥18 months, even after starting a new anticancer treatment.

### Efficacy Assessment

Tumor assessment will be based on CT/magnetic resonance imaging (MRI) with contrast of the head, neck, chest, abdomen, and appropriate anatomy of tumor location per RECIST v1.1, Q8W after treatment start for up to 12 months. After 12 months, tumor assessments can be conducted Q12W. Brain scans are required at baseline if clinically indicated and, if metastases are present, are repeated at the same frequency as the head, neck, chest, and abdomen scans. Bone scans will be performed as clinically indicated for patients with suspected bone metastases at baseline outside of the CT scan regions, or suspected lesions on study. Imaging of all patients will undergo BICR by a central imaging center and local investigator review.

### Concomitant Medications

### Permitted

Petosemtamab is catabolized by lysosomal enzymes in the liver and/or kidney into amino acids that are then reabsorbed and/or reincorporated into endogenous proteins. Therefore, no drug-drug interactions are expected with drugs metabolized by cytochrome P450 (CYP450), including components of the FOLFIRI and FOLFOX regimens.

Suitable recommended premedication regimen should be followed before the first infusion. At subsequent infusions, it is recommended that the investigators administer appropriate premedication regimen at their discretion, using their medical judgment. In the event of IRRs, hypersensitivity or allergic reactions, symptomatic treatment should be administered according to standard local clinical practice, and dosing instructions should be followed.

All medication necessary for the patient's safety and wellbeing, and which is not expected to interfere with evaluation of the study drug, may be given at the investigator's discretion.

Concurrent radiation treatment during this study for symptom control without evidence of progression, only with authorization of the Sponsor.

Prohibited medications include concomitant chronic oral corticosteroids (>10 mg/day prednisone equivalent), tumor necrosis factor (TNF-alpha inhibitors, anti-T-cell antibodies, or other immunosuppressive medication. No strong CYP3A4 inducers, strong CYP3A4 or UGT1A1 inhibitors are to be administered with irinotecan unless there are no therapeutic alternatives. Any investigational drug or other anticancer therapy during the study. Optionally, initiation of herbal remedies for cancer treatment. Herbal remedies initiated prior to study entry and continuing during the study are permitted. Major surgery or radiotherapy without Sponsor consent unless in emergency. Prior radiotherapy to ≥25% of bone marrow.

1. An antibody or functional part, derivative and/or analogue thereof that comprises a variable domain that binds an extracellular part of EGFR, for use in the treatment of a cancer in a subject, wherein the treatment further comprises administering a fluoropyrimidine, a platinum-based chemotherapeutic agent, a BCL-2 inhibitor, or SN-38.

2. A method of treating cancer in a subject, the method comprising administering an effective amount of an antibody or functional part, derivative and/or analogue thereof that comprises a variable domain that binds an extracellular part of EGFR, and a fluoropyrimidine, a platinum-based chemotherapeutic agent, a BCL-2 inhibitor, or SN-38, to the subject.

3. The antibody or functional part, derivative and/or analogue thereof, or the method of claim 1 or 2, wherein the cancer is an adenocarcinoma or a squamous cell carcinoma.

4. The antibody or functional part, derivative and/or analogue thereof, or the method of any one of the preceding claims, wherein the cancer is a head and neck cancer, gastric, esophageal, gastro-esophageal-junction cancer, non-small cell lung cancer or colorectal cancer.

5. The antibody or functional part, derivative and/or analogue thereof, or the method of any one of the preceding claims, wherein the cancer is a colorectal cancer.

6. The antibody or functional part, derivative and/or analogue thereof, or the method of any one of the preceding claims, wherein the fluoropyrimidine is fluorouracil.

7. The antibody or functional part, derivative and/or analogue thereof, or the method of claim 6, wherein fluorouracil is administered in a range of 200-3000 mg/m² intravenously.

8. The antibody or functional part, derivative and/or analogue thereof, or the method of claim 6 or 7, wherein fluorouracil is administered at 400 mg/m² by intravenous bolus on Day 1, followed by 2400 mg/m² to 3000 mg/m² intravenously as a continuous infusion over 46 hours every two weeks.

9. The antibody or functional part, derivative and/or analogue thereof, or the method of claims 6 or 7, wherein fluorouracil is administered at 500 mg/m² by intravenous bolus on Days 1, 8, 15, 22, 29, and 36 in 8-week cycles.

10. The antibody or functional part, derivative and/or analogue thereof, or the method of any one of claims 1-5, wherein the fluoropyrimidine is trifluridine and the treatment further comprises administering a thymidine phosphorylase inhibitor.

11. The antibody or functional part, derivative and/or analogue thereof, or the method of claim 10, wherein the thymidine phosphorylase inhibitor is tipiracil hydrochloride.

12. The antibody or functional part, derivative and/or analogue thereof, or the method of claim 10 or 11, wherein the trifluridine and tipiracil hydrochloride are administered at a molar ratio of 1:0.5 at 35-80 mg/m² twice daily.

13. The antibody or functional part, derivative and/or analogue thereof, or the method of any one of the claims 1-5, wherein the platinum-based chemotherapeutic agent is oxaliplatin.

14. The antibody or functional part, derivative and/or analogue thereof, or the method of claim 13, wherein oxaliplatin is administered at 85 mg/m² every two weeks.

15. The antibody or functional part, derivative and/or analogue thereof, or the method of any one of claims 1-5, wherein the BCL-2 inhibitor is venetoclax.

16. The antibody or functional part, derivative and/or analogue thereof, or the method of claim 15, wherein venetoclax is administered at 20-1200 mg daily.

17. The antibody or functional part, derivative and/or analogue thereof, or the method of any one of the claims 1-5, wherein the treatment comprises administering SN-38.

18. The antibody or functional part, derivative and/or analogue thereof, or the method of claim 6, wherein the treatment further comprises administering of oxaliplatin and folinic acid.

19. The antibody or functional part, derivative and/or analogue thereof, or the method of claim 18, wherein oxaliplatin is administered at 50-200 mg/m² together with folinic acid at 200-600 mg/m² intravenously, followed by a fluorouracil at 1200-3600 mg/m² intravenously.

20. The antibody or functional part, derivative and/or analogue thereof, or the method of claim 6, wherein the treatment further comprises administering of irinotecan and folinic acid.

21. The antibody or functional part, derivative and/or analogue thereof, or the method of claim 20, wherein irinotecan is administered at 180 mg/m² together with folinic acid at 200-400 mg/m² intravenously, followed by fluorouracil at 400-2400 mg/m² intravenously.

22. The antibody or functional part, derivative and/or analogue thereof, or the method of any one of the preceding claims, wherein the antibody is ADCC enhanced.

23. The antibody or functional part, derivative and/or analogue thereof, or the method of any one of the preceding claims, wherein the antibody is afucosylated.

24. The antibody or functional part, derivative and/or analogue thereof, or the method of any one of the preceding claims, wherein the antibody is a multispecific antibody.

25. The antibody or functional part, derivative and/or analogue thereof, or the method of any one of the preceding claims, wherein the antibody is a bispecific antibody.

26. The antibody or functional part, derivative and/or analogue thereof, or the method of any one of the preceding claims, wherein the variable domain that binds an extracellular part of EGFR is a heavy chain variable region that comprises:
at least the CDR3 sequence of the VH of MF3370; MF3755; MF4280 or MF4289 as depicted in FIG. 3 or a CDR3 sequence that differs in at most three, two, or one amino acid from the CDR3 sequence of the VH of MF3370; MF3755; MF4280 or MF4289 as depicted in FIG. 3; or
at least the CDR1, CDR2 and CDR3 sequences of the VH of MF3370; MF3755; MF4280 or MF4289 as depicted in FIG. 3; or the CDR1, CDR2 and CDR3 sequences of the VH of MF3370; MF3755; MF4280 or MF4289 as depicted in FIG. 3 with at most three, two, or one amino acid substitutions; or
the amino acid sequence of the VH chain of MF3370; MF3755; MF4280 or MF4289 as depicted in FIG. 3; or
the amino acid sequence of the VH chain of MF3370; MF3755; MF4280 or MF4289 depicted in FIG. 3 having at most 15, or 1, 2, 3, 4, 5, 6, 7, 8, 9 or 10 amino acid insertions, deletions, substitutions or a combination thereof with respect to the VH chain of MF3370; MF3755; MF4280 or MF4289.

27. The antibody or functional part, derivative and/or analogue thereof, or the method of any one of the preceding claims, wherein the antibody comprises a second variable domain that does not bind EGFR.

28. The antibody or functional part, derivative and/or analogue thereof, or the method of any one of the preceding claims, wherein the antibody comprises a second variable domain that binds LGR5.

29. The antibody or functional part, derivative and/or analogue thereof, or the method of any one of the preceding claims, wherein the variable domain that binds LGR5 binds an epitope that is located within amino acid residues 21-118 of the human LGR5 sequence depicted in FIG. 1.

30. The antibody or functional part, derivative and/or analogue thereof, or the method of any one of the preceding claims, wherein the variable domain that binds LGR5 is a heavy chain variable region that comprises:
at least the CDR3 sequence of the VH of MF5790; MF5803; MF5805; MF5808; MF5809; MF5814; MF5816; MF5817; or MF5818 as depicted in FIG. 3 or a CDR3 sequence that differs in at most three, two, or one amino acid from a CDR3 sequence of the VH of MF5790; MF5803; MF5805; MF5808; MF5809; MF5814; MF5816; MF5817; or MF5818 as depicted in FIG. 3; or
at least the CDR1, CDR2 and CDR3 sequences of the VH of MF5790; MF5803; MF5805; MF5808; MF5809; MF5814; MF5816; MF5817; or MF5818 as depicted in FIG. 3; or the CDR1, CDR2 and CDR3 sequences of the VH of MF5790; MF5803; MF5805; MF5808; MF5809; MF5814; MF5816; MF5817; or MF5818 as depicted in FIG. 3 with at most three, two, or one amino acid substitutions; or
the sequence of the VH chain of MF5790; MF5803; MF5805; MF5808; MF5809; MF5814; MF5816; MF5817; or MF5818 as depicted in FIG. 3; or
the amino acid sequence of the VH chain of MF5790; MF5803; MF5805; MF5808; MF5809; MF5814; MF5816; MF5817; or MF5818 as depicted in FIG. 3 having at most 15, or 1, 2, 3, 4, 5, 6, 7, 8, 9 or 10 amino acid insertions, deletions, substitutions or a combination thereof with respect to the VH chain of MF5790; MF5803; MF5805; MF5808; MF5809; MF5814; MF5816; MF5817; or MF5818.

31. The antibody or functional part, derivative and/or analogue thereof, or the method of any one of the preceding claims, wherein the antibody is petosemtamab.

32. The antibody or functional part, derivative and/or analogue thereof, or the method of any one of claims 1-26, wherein the antibody is a monovalent antibody that does not comprise a second variable domain or wherein the antibody comprises said EGFR binding variable domain as the only variable domain.

33. The antibody or functional part, derivative and/or analogue thereof, or the method of any one of the preceding claims, wherein the subject is a mammal, in particular a human.

34. The antibody or functional part, derivative and/or analogue thereof, or the method of any one of the preceding claims, wherein said treatment comprises providing 1500 mg of the antibody or functional part, derivative and/or analogue thereof to the subject.

35. The antibody or functional part, derivative and/or analogue thereof, or the method of any one of the preceding claims, wherein the subject to be treated has not received prior anti-cancer therapy.

36. The antibody or functional part, derivative and/or analogue thereof, or the method of any one of claims 1-34, wherein the cancer or subject to be treated has progressed after prior anti-cancer therapy.

37. The antibody or functional part, derivative and/or analogue thereof, or the method of claim 36, wherein the prior anti-cancer therapy is chemotherapy, targeted therapy, immunotherapy or radiation therapy.

38. The antibody or functional part, derivative and/or analogue thereof, or the method of claim 36 or 37, wherein the prior anti-cancer therapy is fluorouracil, a platinum-based chemotherapeutic agent, oxaliplatin, FOLFOX, FOLFIRI, TAS-102,
trastuzumab, pembrolizumab, nivolumab, cetuximab or a combination thereof.

39. The antibody or functional part, derivative and/or analogue thereof, or the method of any one of the preceding claims, wherein the antibody or functional part, derivative and/or analogue thereof and the fluoropyrimidine, the platinum-based chemotherapeutic agent, the BCL-2 inhibitor, or SN-38 are administered to the subject simultaneously, separately, or sequentially.

40. The method of treating cancer in a subject of any one of the preceding claims, wherein the method comprises administering an effective amount of the antibody or functional part, derivative and/or analogue thereof, and an effective amount of the fluoropyrimidine, platinum-based chemotherapeutic agent, BCL-2 inhibitor, or SN-38, to the subject.

41. A pharmaceutical composition comprising an antibody or functional part, derivative and/or analogue thereof as defined in any one of the preceding claims and a fluoropyrimidine, a platinum-based chemotherapeutic agent, a BCL-2 inhibitor, or SN-38.

42. The pharmaceutical composition according to claim 41, wherein the antibody or functional part, derivative and/or analogue thereof, and the fluoropyrimidine, platinum-based chemotherapeutic agent, a BCL-2 inhibitor, or SN-38 are provided in a single formulation.

43. The pharmaceutical composition according to claim 41, wherein the antibody or functional part, derivative and/or analogue thereof, and the fluoropyrimidine, a platinum-based chemotherapeutic agent, a BCL-2 inhibitor, or SN-38 are provided in separate formulation.

44. A kit of parts comprising
an antibody or functional part, derivative and/or analogue thereof, as defined in any one of the preceding claims;
a fluoropyrimidine, a platinum-based chemotherapeutic agent, a BCL-2 inhibitor, or SN-38; and
instructions for use of said antibody or functional part, derivative and/or analogue thereof and instructions for use of said fluoropyrimidine, platinum-based chemotherapeutic agent, BCL-2 inhibitor, or SN-38.

45. The kit of parts, according to claim 44, wherein the instructions for use of the antibody or functional part, derivative/and or analogue thereof comprises instructions for dosing thereof at 1500 mg.

46. The kit of parts according to claim 44 or 45, wherein the kit comprises instructions for use of the antibody or functional part, derivative and/or analogue thereof and fluoropyrimidine, platinum-based chemotherapeutic agent, BCL-2 inhibitor, or SN-38, in the treatment of adenocarcinoma or a squamous cell carcinoma.

47. The kit of parts according to any one of claims 44-46, wherein the kit comprises instructions for use of the antibody or functional part, derivative and/or analogue thereof and fluoropyrimidine, platinum-based chemotherapeutic agent, BCL-2 inhibitor, or SN-38, in the treatment of head and neck cancer, gastric, esophageal, gastro-esophageal-junction cancer, non-small cell lung cancer or colorectal cancer.

48. The kit of parts according to any one of claims 44-47, wherein the kit comprises instructions for use of the antibody or functional part, derivative and/or analogue thereof and fluoropyrimidine, platinum-based chemotherapeutic agent, BCL-2 inhibitor, or SN-38, in the treatment of colorectal cancer.

49. A combination of an antibody or functional part, derivative and/or analogue thereof as defined in any one of the preceding claims, and a fluoropyrimidine, platinum-based chemotherapeutic agent, BCL-2 inhibitor, or SN-38 as defined in any one of the preceding claims for use in the treatment of cancer in a subject in need thereof.

50. A combination of
an antibody or functional part, derivative and/or analogue thereof, as defined in any one of the preceding claims,
instructions for use in the treatment of a cancer in a subject of said antibody, and
instructions for use in the treatment of a cancer in a subject of a fluoropyrimidine, a platinum-based chemotherapeutic agent, a BCL-2 inhibitor, or SN-38 as defined in any one of the preceding claims.

51. The combination of claim 50, wherein the instructions for use comprise the amount of the antibody or functional part, derivative and/or analogue thereof and/or the amount of the fluoropyrimidine, platinum-based chemotherapeutic agent, BCL-2 inhibitor, or SN-38 to be used, and/or the dosing interval and/or the cancer to be treated.

52. A combination of
a fluoropyrimidine, a platinum-based chemotherapeutic agent, a BCL-2 inhibitor, or a SN-38, as defined in any one of the preceding claims,
instructions for use in the treatment of a cancer in a subject of a fluoropyrimidine, a platinum-based chemotherapeutic agent, a BCL-2 inhibitor, or a SN-38, and
instructions for use in the treatment of a cancer in a subject of an antibody or functional part, derivative and/or analogue thereof, as defined in any one of the preceding claims.

53. The combination of claim 52, wherein the instructions for use comprise the amount of the antibody or functional part, derivative and/or analogue thereof and/or the amount of the fluoropyrimidine, platinum-based chemotherapeutic agent, BCL-2 inhibitor, or SN-38 to be used, and/or the dosing interval and/or the cancer to be treated.

54. The use of an antibody or functional part, derivative and/or analogue thereof as defined in any one of the preceding claims and a fluoropyrimidine, a platinum-based chemotherapeutic agent, a BCL-2 inhibitor, or SN-38, in the manufacture of one or more medicaments, for the treatment of a cancer in a subject, wherein treatment comprises administering the antibody or functional part, derivative and/or analogue thereof and the fluoropyrimidine, the platinum-based chemotherapeutic agent, the BCL-2 inhibitor, or SN-38, simultaneously, separately, or sequentially.
